(19) United States
(12) Patent Application Publication
(10) Pub. No.: US 2023/0159955 A1
(43) Pub. Date: May 25, 2023

(54) CIRCULAR-PERMUTED NUCLEIC ACIDS FOR HOMOLOGY-DIRECTED EDITING

(71) Applicant: Zymergen Inc., Emeryville, CA (US)

(72) Inventors: Kunal MEHTA, Oakland, CA (US); Philip WEYMAN, Alameda, CA (US); Solomon STONEBLOOM, Alameda, CA (US)

(21) Appl. No.: 17/918,525

(22) PCT Filed: Apr. 16, 2021
(86) PCT No.: PCT/US2021/027689

§ 371 Date: Oct. 12, 2022

Related U.S. Application Data
(60) Provisional application No. 63/010,871, filed on Apr. 16, 2020.

(51) Int. Cl. C12N 15/90; C12N 15/10; C12N 15/81

(52) U.S. Cl. CPC C12N 15/905; C12N 15/102; C12N 15/81; C12N 2310/20

(57) ABSTRACT

The present disclosure relates to compositions and methods of generating circular-permuted nucleic acids for direct use in homology directed genome editing. The method also allows the joining of a large number of DNA fragments, in a deterministic fashion. It can be used to rapidly generate nucleic acid libraries that can be directly used in a variety of applications without further cloning steps that include, for example, genome editing and pathway assembly. Kits for performing the method are also disclosed.

### CROSS-REFERENCE TO RELATED APPLICATIONS

This application claims the benefit of priority to U.S. Provisional Application Ser. No. 63/010,871, filed Apr. 16, 2020, which is herein incorporated by reference in its entirety for all purposes.

### FIELD

The present disclosure is directed to compositions and methods for joining single-stranded and/or double-stranded nucleic acid molecules permitting in vitro or in vivo assembly of multiple nucleic acid molecules with overlapping terminal sequences in a single reaction that are suitable for genetic editing of host cell genomes without further assembly or cloning steps. The disclosed methods and compositions can be useful for deterministic assembly of fragments of nucleic acid sequences that can be directly used for editing any DNA sequence such as, for example, plasmids, cosmid or specific genes in the genome of desired host cells or organisms.

### BACKGROUND

Traditionally, nucleic acid assemblies such as plasmid or linear DNA are generated one at a time in a deterministic fashion and, thus, can be slow, expensive and labor-intensive. In contrast, current pooled approaches for generating libraries of complex nucleic acid assemblies can enable the generation of many assemblies at once, but often result in libraries representing all possible combinations between the sets of parts in the assembly. Such approaches are a non-deterministic and combinatorial approach to assembly and can also be time-consuming, labor intensive and expensive, especially in circumstances where a subset of sequences is the desired product of the assembly reaction.

Thus, there is a need in the art for new methods for generating complex nucleic acid assemblies that can be used directly (i.e., without the need for further assembly or cloning into constructs that contain sequences that target the assemblies to genetic loci) as integration fragments for genetic editing, which do not suffer from the aforementioned drawbacks inherent with traditional methods for generating nucleic acid assemblies.

SUMMARY

In one aspect, provided herein is a method for genetically editing a host cell, the method comprising: (a) assembling a pool of insert polynucleotides and a pool of targeting polynucleotides into a pool of circular molecules, wherein each circular molecule from the pool of circular molecules comprises one or more payload sequences flanked by a first homology arm 5′ to the one or more payload sequences and a second homology arm 3′ to the one or more payload sequences and a linearization sequence that is located between both the first and second homology arms; (b) linearizing each of the circular molecules from the pool of circular molecules via the linearization sequence present on each circular molecule, thereby generating a pool of linear insert polynucleotides, wherein each linear insert polynucleotide in the pool comprises from 5′ to 3′ a first homology arm, one or more payload sequences and a second homology arm, wherein the first homology arm and the second homology arm comprise sequence complementary to a genomic locus in a host cell; and (c) introducing the pool of linear insert polynucleotides into the host cell, thereby genetically editing the host cell.

In some cases, the assembling of step (a) comprises: (i) providing a pool of reverse primers along with the pool of insert polynucleotides and the pool of targeting polynucleotides, wherein the pool of targeting polynucleotides act as forward primers, thereby generating a mixture comprising the pool of insert polynucleotides, the pool of forward primers and the pool of reverse primers, wherein, for each insert polynucleotide, the mixture comprises at least one forward primer from the pool of forward primers and a reverse primer from the pool of reverse primers, wherein the at least one forward primer comprises from 5′ to 3′, a first assembly overlap sequence comprising sequence complementary to a distal or 3′ end of the insert polynucleotide, the first homology arm, the linearization sequence, the second homology arm and a second assembly overlap sequence comprising sequence complementary to a reverse complement of a proximal or 5′ end of the insert polynucleotide, and wherein the reverse primer comprises sequence complementary to the distal or 3′ end of the insert polynucleotide; (ii) performing a polymerase chain reaction (PCR) on the mixture, wherein, for each insert polynucleotide, the PCR generates a PCR product comprising from 5′ to 3′, the first assembly overlap sequence, the first homology arm, the linearization sequence, the second homology arm and the one or more payload sequences; and (iii) circularizing the PCR products from step (ii) via an assembly method selected from the group consisting of splicing and overlap-extension PCR (SOE-PCR), Uracil-specific excision reagent (USER) cloning, restriction-ligation, scarless restriction-ligation, blunt-end ligation, an overlap based assembly method and a recombination-based method, or any other enzymatic or chemical method of joining two DNA molecules. In some cases, the first assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the distal or 3′ end of the insert polynucleotide. In some cases, the second assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the reverse complement of the proximal or 5′ end of the insert polynucleotide. In some cases, the distal or 3′ end of the insert polynucleotide to which the first assembly overlap sequence comprises sequence complementary thereto is found within one of the one or more payload sequences. In some cases, the distal or 3′ end of the insert polynucleotide to which the first assembly overlap sequence comprises sequence complementary thereto is found downstream of the one or more payload sequences. In some cases, the proximal or 5′ end of the insert polynucleotide to which the second assembly overlap sequence comprises sequence complementary to the reverse complement thereof is found within one of the one or more payload sequences. In some cases, the proximal or 5′ end of the insert polynucleotide to which the second assembly overlap sequence comprises sequence complementary to the reverse complement thereof is found upstream of the one or more payload sequences. In some cases, the distal or 3′ end of the insert polynucleotide to which the reverse primer comprises sequence complementary thereto is found within one of the one or more payload sequences. In some cases, the distal or 3′ end of the insert polynucleotide to which the reverse primer comprises sequence complementary thereto is found downstream of the one or more payload sequences.

In some cases, the assembling of step (a) comprises directly performing an assembly method on a mixture comprising the pool of insert polynucleotides and the pool of targeting polynucleotides, wherein, for each insert polynucleotide, the mixture comprises at least one targeting polynucleotide from the pool of targeting polynucleotides, wherein the at least one targeting polynucleotide comprises from 5′ to 3′, a first assembly overlap sequence comprising sequence complementary to a distal or 3′ end of the insert polynucleotide, the first homology arm, the linearization sequence, the second homology arm and a second assembly overlap sequence comprising sequence complementary to reverse complement of a proximal or 5′ end of the insert polynucleotide, and wherein the assembly method is selected from selected from the group consisting of splicing and overlap-extension PCR (SOE-PCR), Uracil-specific excision reagent (USER) cloning, restriction-ligation, blunt-end ligation, overlap based assembly method and recombination-based method, or any other enzymatic or chemical method of joining two DNA molecules. In some cases, the assembling method is an overlap based assembly method utilizing a Type IISIIS restriction enzyme and a ligase, wherein each insert polynucleotide in the pool of insert polynucleotides comprises a recognition sequence for the Type IISIIS restriction enzyme on both the insert polynucleotide's proximal or 5′ end and distal or 3′ end, which, upon digestion with the Type IISIIS restriction enzyme, generates a proximal overhang and distal overhang, respectively, and wherein, for each insert polynucleotide, the mixture comprises at least one targeting polynucleotide from the pool of targeting polynucleotides, wherein the first assembly overlap sequence and the second assembly overlap sequence of the at least one targeting polynucleotide each comprise the recognition sequence for the Type IISIIS restriction enzyme which, upon digestion with the Type IIS restriction enzyme, generates an overhang in the first assembly overlap sequence compatible with the distal overhang of the insert polynucleotide as well as an overhang in the second assembly overlap sequence compatible with the proximal overhang of the insert polynucleotide. In some cases, the Type IIS restriction enzyme is a Type IIS restriction enzyme that generates a four-base overhang. In some cases, the Type IIS restriction enzyme is selected from the group consisting of BsaI, BbsI, BsmBI and Esp3I. In some cases, the ligase is a T4 DNA ligase. In some cases, each targeting polynucleotide in the pool of targeting polynucleotides is subjected to a primer extension reaction using a reverse primer comprising sequence that binds to the second assembly overlap sequence, thereby generating a double-stranded (ds) targeted polynucleotide. In some cases, the top or sense strand of each ds targeting polynucleotide comprises, from 5′ to 3′, the first assembly overlap sequence comprising sequence complementary to the distal or 3′ end of the insert polynucleotide, the first homology arm, the linearization sequence, the second homology arm and the second assembly overlap sequence comprising sequence complementary to the reverse complement of the proximal or 3′ end of the insert polynucleotide. In some cases, the first assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the distal or 3′ end of the insert polynucleotide. In some cases, the second assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the reverse complement of the proximal or 5′ end of the insert polynucleotide. In some cases, the distal or 3′ end of the insert polynucleotide to which the first assembly overlap sequence comprises sequence complementary thereto is found within one of the one or more payload sequences. In some cases, the distal or 3′ end of the insert polynucleotide to which the first assembly overlap sequence comprises sequence complementary thereto is found downstream of the one or more payload sequences. In some cases, the proximal or 5′ end of the insert polynucleotide to which the second assembly overlap sequence comprises sequence complementary to the reverse complement thereof is found within one of the one or more payload sequences. In some cases, the proximal or 5′ end of the insert polynucleotide to which the second assembly overlap sequence comprises sequence complementary to the reverse complement thereof is found upstream of the one or more payload sequences. In some cases, the distal or 3′ end of the insert polynucleotide to which the reverse primer comprises sequence complementary thereto is found within one of the one or more payload sequences. In some cases, the distal or 3′ end of the insert polynucleotide to which the reverse primer comprises sequence complementary thereto is found downstream of the one or more payload sequences.

In some cases, the linearizing of step (b) comprises rolling circle amplification (RCA) of each circular molecule from the pool of circular molecules, wherein the RCA of each circular molecule produces a concatenated linear product comprising repeated units each separated by the linearization sequence, wherein each of the repeated units comprises the insert polynucleotide flanked upstream by the first homology arm and downstream by the second homology arm, wherein the insert polynucleotides are released from the concatenated linear product via the linearization sequence present between each repeated unit, thereby generating the pool of linear insert polynucleotides.

In some cases, the linearization sequence comprises one or more recognition sequences for one or more site-specific nucleases. In some cases, the linearizing of step (b) comprises digesting the one or more recognition sequences (in either the circularized molecules or the concatenated linear product) with one or more site-specific nuclease(s) that recognize the one or more site-specific nuclease recognition sequence(s). In some cases, the one or more site-specific nuclease(s) recognition sequence are for one or more of Type I restriction endonuclease(s), Type IIS restriction endonuclease(s), meganuclease, RNA-guided nuclease(s), DNA-guided nuclease(s), zinc-finger nuclease(s), TALEN(s) or nicking enzyme(s).

In some cases, the linearization sequence comprises one or more primer binding sites that are common to each targeting polynucleotide in the pool of targeting polynucleotides. In some cases, the linearizing of step (b) comprises performing a PCR using a primer pair directed to one of the one or more primer binding sites located within the linearization sequence. In some cases, at least one of the one or more primer binding sites in the targeting polynucleotide is common to at least one of the one or more primer binding sites in each other targeting polynucleotide in the pool of targeting polynucleotides. In some cases, the primer pair directed to one of the one or more primer binding sites within the linearization sequence in step (b) is directed to the primer binding site common to each targeting polynucleotide in the pool of targeting polynucleotides. In some cases, at least one of the one or more primer binding sites in the targeting polynucleotide is not found in any of the one or more primer binding sites in each other targeting polynucleotide in the pool of targeting polynucleotides. In some cases, the primer pair directed to one of the one or more primer binding sites located within the linearization sequence in step (b) is directed to the primer binding site not found in any of the one or more primer binding sites in each other targeting polynucleotide in the pool of targeting polynucleotides. In some cases, at least one of the one or more primer binding sites in the targeting polynucleotide is common to at least one of the one or more primer binding sites in a subset of other targeting polynucleotides in the pool of targeting polynucleotides. In some cases, the primer pair directed to one of the one or more primer binding sites located within the linearization sequence in step (b) is directed to the primer binding site common to the subset of other targeting polynucleotides in the pool of targeting polynucleotides.

In some cases, each insert polynucleotide is present on a plasmid. In some cases, each insert polynucleotide is a linear fragment of nucleic acid. In some cases, each insert polynucleotide is single-stranded or double-stranded. In some cases, each linear insert polynucleotide is a gBlock. In some cases, each payload sequence is selected from the group consisting of whole or portions of promoters, genes, regulatory sequences, nucleic acid sequence encoding degrons, nucleic acid sequence encoding solubility tags, terminators, unique identifier sequence, and combinations thereof. In some cases, each payload sequence and/or targeting polynucleotide comprises a barcode sequence. In some cases, the barcode sequence comprises a sequence unique to each combination of payload sequence and first and second homology arms flanked by sequence universal to the barcode sequence present in each other payload sequence. In some cases, the sequence universal to the barcode sequence present in each other payload sequence is used for amplifying or sequencing the unique sequence in each barcode. In some cases, the insert polynucleotide further comprises sequence for a selectable marker. In some cases, the sequence for the selectable marker is flanked by direct repeat sequences that serve to facilitate looping out of the sequence for the selectable marker. In some cases, the selectable marker is selected from the group consisting of an antibiotic resistance gene, an auxotrophic marker, a colorimetric marker, a gene for a reporter protein and a directional marker. In some cases, the first and second homology arms on each circular molecule comprise sequence corresponding to a different genomic locus in the host cell as compared to each other first and second homology arms on each other circular molecule. In some cases, the first and second homology arms on each circular molecule comprise sequence corresponding to the same genomic locus in the host cell as compared to each other first and second homology arms on each other circular molecule. In some cases, each of the one or more payload sequences in a circular molecule is different from the one or more payload sequences in each other circular molecule. In some cases, each of the one or more payload sequences in a circular molecule is the same as the one or more payload sequences in each other circular molecule.

In some cases, the introducing in step (c) entails performing double-crossover integration of the pool of linear insert polynucleotides in the host cell. In some cases, the introducing in step (c) entails performing CRISPR-mediated homology directed repair with the pool of linear insert polynucleotides and a pool of guide RNAs (gRNA) introduced into the host cell. In some cases, each of the gRNAs in the pool of gRNAs comprise sequence complementary to a genomic locus targeted by the first and second homology arms in one or more of the linear insert polynucleotides present in the pool of linear insert polynucleotides. In some cases, the pool of gRNAs comprises gRNAs that target or bind the genomic loci targeted by each of the linear insert polynucleotides in the pool of linear insert polynucleotides. In some cases, the pool of gRNAs comprises gRNAs that target or bind genomic loci targeted by a subset of linear insert polynucleotides in the pool of linear insert polynucleotides. In some cases, the introducing in step (c) entails performing lambda red mediated integration of the pool of linear insert polynucleotides in the host cell. In some cases, the host cell is selected from the group consisting of a bacterial cell, an algal cell, a plant cell, a fungal cell, an insect cell and a mammalian cell. In some cases, the host cell is a bacterial cell. In some cases, the bacterial cell is selected from Escherichia coli and Corynebacterium glutamicum. In some cases, the Corynebacterium glutamicum is selected from Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866. In some cases, the Escherichia coli is selected from Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli O157:H7, E. coli O104:H4, Escherichia coli O121, Escherichia coli O104:H21, Escherichia coli K1, and Escherichia coli NC101. In some cases, the host cell is a fungal cell. In some cases, the fungal cell is selected from Saccharomyces cerevisiae and Pichia pastoris. In some cases, the fungal cell is a filamentous fungal cell. In some cases, the filamentous fungal cell is selected from Achlya, Acremonium, Aspergillus, Aureobasidium, Bjerkandera, Ceriporiopsis, Cephalosporium, Chrysosporium, Cochhobolus, Corynascus, Cryphonectria, Cryptococcus, Coprinus, Coriolus, Diplodia, Endothis, Fusarium, Gibberella, Ghocladium, Humicola, Hypocrea, Mycehophthora (e.g., Mycehophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella species or teleomorphs, or anamorphs, and synonyms or taxonomic equivalents thereof. In some cases, the filamentous fungal host cell is Aspergillus niger.

In another aspect, provided herein is a composition comprising a pool of insert polynucleotides, and a pool of targeting polynucleotides, wherein each insert polynucleotide in the pool of insert polynucleotides comprises one or more payload sequences, wherein, for each insert polynucleotide, the composition comprises at least one targeting polynucleotide from the pool of targeting polynucleotides, wherein the at least one targeting polynucleotide comprises from 5′ to 3′, a first assembly overlap sequence comprising sequence complementary to a distal or 3′ end of the insert polynucleotide, a first homology arm, a linearization sequence, a second homology arm and a second assembly overlap sequence comprising sequence complementary to a reverse complement of a proximal or 5′ end of the insert polynucleotide, wherein the first homology arm and the second homology arm comprise sequence complementary to a genomic locus in a host cell. In some cases, the composition further comprises a pool of reverse primers, wherein, for each insert polynucleotide, the composition comprises at least one targeting polynucleotide from the pool of targeting polynucleotides and a reverse primer from the pool of reverse primers, wherein the at least one targeting polynucleotide comprises from 5′ to 3′, a first assembly overlap sequence comprising sequence complementary to a distal or 3′ end of the insert polynucleotide, the first homology arm, the linearization sequence, the second homology arm and a second assembly overlap sequence comprising sequence complementary to a reverse complement of a proximal or 5′ end of the insert polynucleotide, and wherein the reverse primer comprises sequence complementary to the distal or 3′ end of the insert polynucleotide, and wherein the pool of targeting polynucleotides is a pool of forward primers. In some cases, each insert polynucleotide in the pool of insert polynucleotides comprises a recognition sequence for the Type IIS restriction enzyme on both the insert polynucleotide'sproximal or 5′ end and distal or 3′ end, which upon digestion with the Type IIS restriction enzyme, generates a proximal overhang and distal overhang, respectively, and wherein, for each insert polynucleotide, the mixture comprises at least one targeting polynucleotide from the pool of targeting polynucleotides, wherein the first assembly overlap sequence and the second assembly overlap sequence of the at least one targeting polynucleotide each comprise the recognition sequence for the Type IIS restriction enzyme, which, upon digestion with the Type IIS restriction enzyme, generates an overhang in the first assembly overlap sequence compatible with the distal overhang of the insert polynucleotide as well as an overhang in the second assembly overlap sequence compatible with the proximal overhang of the insert polynucleotide. In some cases, the composition further comprises a Type IIS restriction enzyme and a ligase. In some cases, the Type IIS restriction enzyme is a Type IIS restriction enzyme that generates a four-base overhang. In some cases, the Type IIS restriction enzyme is selected from the group consisting of Bsal, Bbsl, BsmBI and Esp3I. In some cases, the ligase is a T4 DNA ligase. In some cases, the linearization sequence comprises one or more recognition sequences for one or more site-specific nucleases. In some cases, the one or more site-specific nuclease(s) recognition sequence are for one or more of Type I restriction endonuclease(s), Type

IIS restriction endonuclease(s), a meganuclease, RNA-guided nuclease(s), DNA-guided nuclease(s), zinc-finger nuclease(s), TALEN(s) or nicking enzyme(s). In some cases, the linearization sequence comprises one or more primer binding sites that are common to each targeting polynucleotide in the pool of targeting polynucleotides. In some cases, at least one of the one or more primer binding sites in the targeting polynucleotide is common to at least one of the one or more primer binding sites in each other targeting polynucleotide in the pool of targeting polynucleotides. In some cases, the primer pair directed to one of the one or more primer binding sites located within the linearization sequence is directed to the primer binding site common to each targeting polynucleotide in the pool of targeting polynucleotides. In some cases, at least one of the one or more primer binding sites in the targeting polynucleotide is not found in any of the one or more primer binding sites in each other targeting polynucleotide in the pool of targeting polynucleotides. In some cases, the primer pair directed to one of the one or more primer binding sites located within the linearization sequence is directed to the primer binding site not found in any of the one or more primer binding sites in each other targeting polynucleotide in the pool of targeting polynucleotides. In some cases, at least one of the one or more primer binding sites in the targeting polynucleotide is common to at least one of the one or more primer binding sites in a subset of other targeting polynucleotides in the pool of targeting polynucleotides. In some cases, the primer pair directed to one of the one or more primer binding sites located within the linearization sequence is directed to the primer binding site common to the subset of other targeting polynucleotides in the pool of targeting polynucleotides. In some cases, the first assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the distal or 3′ end of the insert polynucleotide. In some cases, the second assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the reverse complement of the proximal or 5′ end of the insert polynucleotide. In some cases, the distal or 3′ end of the insert polynucleotide to which the first assembly overlap sequence comprises sequence complementary thereto is found within one of the one or more payload sequences. In some cases, the distal or 3′ end of the insert polynucleotide to which the first assembly overlap sequence comprises sequence complementary thereto is found downstream of the one or more payload sequences. In some cases, the proximal or 5′ end of the insert polynucleotide to which the second assembly overlap sequence comprises sequence complementary to the reverse complement thereof is found within one of the one or more payload sequences. In some cases, the proximal or 5′ end of the insert polynucleotide to which the second assembly overlap sequence comprises sequence complementary to the reverse complement thereof is found upstream of the one or more payload sequences. In some cases, the distal or 3′ end of the insert polynucleotide to which the reverse primer comprises sequence complementary thereto is found within one of the one or more payload sequences. In some cases, the distal or 3′ end of the insert polynucleotide to which the reverse primer comprises sequence complementary thereto is found downstream of the one or more payload sequences. In some cases, each insert polynucleotide is present on a plasmid. In some cases, each insert polynucleotide is a linear fragment of nucleic acid. In some cases, each linear insert polynucleotide is a gBlock. In some cases, each insert polynucleotide is single-stranded or double-stranded. In some cases, each payload sequence is selected from the group consisting of whole or portions of promoters, genes, regulatory sequences, nucleic acid sequence encoding degrons, nucleic acid sequence encoding solubility tags, terminators, unique identifier sequence and combinations thereof. In some cases, each payload sequence and/or targeting polynucleotide comprises a barcode sequence. In some cases, the barcode sequence comprises a sequence unique to each combination of payload sequence and first and second homology arms flanked by sequence universal to the barcode sequence present in each other payload sequence. In some cases, the sequence universal to the barcode sequence present in each other payload sequence is used for amplifying or sequencing the unique sequence in each barcode. In some cases, the insert polynucleotide further comprises sequence for a selectable marker. In some cases, the sequence for the selectable marker is flanked by direct repeat sequences that serve to facilitate looping out of the sequence for the selectable marker. In some cases, the selectable marker is selected from the group consisting of an antibiotic resistance gene, an auxotrophic marker, a colorimetric marker, a gene for a reporter protein and a directional marker. In some cases, the first and second homology arms on each targeting polynucleotide in the pool of targeting polynucleotides comprise sequence corresponding to a different genomic locus in the host cell as compared to each other first and second homology arms on each other targeting polynucleotides in the pool of targeting polynucleotides. In some cases, the first and second homology arms on each targeting polynucleotide in the pool of targeting polynucleotides comprise sequence corresponding to the same genomic locus in the host cell as compared to each other first and second homology arms on each other targeting polynucleotide in the pool of targeting polynucleotides. In some cases, each of the one or more payload sequences in an insert polynucleotide in the pool of insert polynucleotides is different from the one or more payload sequences in each other insert polynucleotide in the pool of insert polynucleotides. In some cases, each of the one or more payload sequences in an insert polynucleotide in the pool of insert polynucleotides is the same as the one or more payload sequences in each other insert polynucleotide in the pool of insert polynucleotides. In some cases, the composition further comprises a pool of guide RNAs (gRNA). In some cases, each of the gRNAs in the pool of gRNAs comprise sequence complementary to a genomic locus targeted by the first and second homology arms in one or more of the target polynucleotides present in the pool of targeting polynucleotides. In some cases, the pool of gRNAs comprises gRNAs that target or bind the genomic loci targeted by each of the target polynucleotides present in the pool of targeting polynucleotides. In some cases, the pool of gRNAs comprises gRNAs that target or bind genomic loci targeted by a subset of target polynucleotides present in the pool of targeting polynucleotides. In some cases, the host cell is selected from the group consisting of a bacterial cell, an algal cell, a plant cell, a fungal cell, an insect cell and a mammalian cell. In some cases, the host cell is a bacterial cell. In some cases, the bacterial cell is selected from Escherichia coli and Corynebacterium glutamicum. In some cases, the Corynebacterium glutamicum is selected from Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866. In some cases, the Escherichia coli is selected from Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli O157:H7, E. coli O104:H4, Escherichia coli O121, Escherichia coli O104:H21, Escherichia coli K1, and Escherichia coli NC101. In some cases, the host cell is a fungal cell. In some cases, the fungal cell is selected from Saccharomyces cerevisiae and Pichia pastoris. In some cases, the fungal cell is a filamentous fungal cell. In some cases, the filamentous fungal cell is selected from Achlya, Acremonium, Aspergillus, Aureobasidium, Bjerkandera, Ceriporiopsis, Cephalosporium, Chrysosporium, Cochhobolus, Corynascus, Cryphonectria, Cryptococcus, Coprinus, Coriolus, Diplodia, Endothis, Fusarium, Gibberella, Ghocladium, Humicola, Hypocrea, Mycehophthora (e.g., Mycehophthora thermophila), Mucor, Neurospora, Penicillium, Podospora, Phlebia, Piromyces, Pyricularia, Rhizomucor, Rhizopus, Schizophyllum, Scyta/idium, Sporotrichum, Talaromyces, Thermoascus, Thielavia, Tramates, Tolypocladium, Trichoderma, Verticillium, Volvariella species or teleomorphs, or anamorphs, and synonyms or taxonomic equivalents thereof. In some cases, the filamentous fungal host cell is Aspergillus niger.

### BRIEF DESCRIPTION OF THE DRAWINGS

FIG. 1A-1C illustrates circular permutation methods for generating homology-directed editing fragments. FIG. 1A shows the design of members in the oligo pool (i.e., targeting polynucleotides). FIG. 1B shows a scheme of the DNA construct containing the payload (i.e., insert polynucleotide). FIG. 1C shows variants of the procedure used to generate the final oligonucleotide pool from the inputs in FIG. 1A and FIG. 1B. Two workflows leading to the circular intermediate are depicted that include (1) using PCR using the targeting polynucleotides as forward primers, the insert polynucleotide as the template (either single or double-stranded) and adding in a pool of reverse primers, followed by circularization via an in vitro assembly method (top portion of FIG. 1C), and (2) direct assembly of the targeting polynucleotides (either single-stranded or after being made double-stranded via primer extension with a supplemented reverse primer) and insert polynucleotides using an in vitro assembly method (bottom portion of FIG. 1C). The circular intermediate can be linearized either by PCR or restriction digest, as indicated.

FIG. 2A-2E illustrates the design, scheme, general procedure and results for double crossover genome editing using circular permuted fragments. FIG. 2A shows design of oligonucleotides in the forward primer pool (i.e., targeting polynucleotide). Fifty-four (54) pairs of unique HomL and HomR sequences were used to target fifty-four (54) loci across the genome. FIG. 2B shows scheme of the final editing fragments after pooled amplification and circular permutation. Edited cells were selected using the URA3 marker. FIG. 2C shows general procedure used to prepare the editing fragments. FIG. 2D shows distribution of genotypes recovered after barcoding. Each bar is a unique genotype corresponding to one of the members of the transformed pool. Twenty-five (25) genotypes were recovered from a total of thirty-six (36) samples analyzed. FIG. 2E shows results from locus-specific sequencing confirmation of barcoded strains.

FIG. 3A-3D illustrates the design, scheme, general procedure and results for CRISPR/Cas9 mediated genome editing using oligo pool derived payloads FIG. 3A shows design of oligonucleotides in the forward primer pool (i.e., targeting polynucleotide). Nine (9) pairs of unique HomL and HomR sequences were used to target nine (9) loci across the genome, results are shown for a single locus. FIG. 3B shows scheme of the final editing fragments after pooled amplification and circular permutation. Potentially edited cells are selected via the natR gene contained on the Cas9 expression plasmid. FIG. 3C shows general procedure used to prepare the editing fragments. FIG. 3D shows the results of structural PCR tested for the integration of the desired edit at ATR1.

FIG. 4 illustrates the Golden Gate Assembly® (i.e., Type IIS restriction enzyme digestion and T4 DNA ligase-based overlap assembly method) based circularization and linearization strategy for the creation of circularized payload sequences.

### DETAILED DESCRIPTION

### Definitions

While the following terms are believed to be well understood by one of ordinary skill in the art, the following definitions are set forth to facilitate explanation of the presently disclosed subject matter.

As used herein, the term “a” or “an” can refer to one or more of that entity, i.e. can refer to a plural referent. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

As used herein, the terms “cellular organism” “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “cellular organisms” or “microbes” of lists/tables and figures present in the disclosure. This characterization can refer to not only the identified taxonomic genera of the tables and figures, but also the identified taxonomic species, as well as the various novel and newly identified or designed strains of any organism in said tables or figures. The same characterization holds true for the recitation of these terms in other parts of the Specification, such as in the Examples.

As used herein, the term “prokaryotes” is art recognized and refers to cells that contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

As used herein, the term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

As used herein, “bacteria” or “eubacteria” can refer to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

As used herein, a “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

As used herein, the terms “genetically modified host cell,” “recombinant host cell,” and “recombinant strain” are used interchangeably herein and can refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell

As used herein, the term “wild-type microorganism” or “wild-type host cell” can describe a cell that occurs in nature, i.e. a cell that has not been genetically modified.

As used herein, the term “genetically engineered” may refer to any manipulation of a host cell's genome (e.g. by insertion, deletion, mutation, or replacement of nucleic acids).

As used herein, the term “control” or “control host cell” can refer to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell. In some embodiments, the present disclosure teaches the use of parent strains as control host cells (e.g., the S₁ strain that was used as the basis for the strain improvement program). In other embodiments, a host cell may be a genetically identical cell that lacks a specific promoter or SNP being tested in the treatment host cell.

As used herein, the term “allele(s)” can mean any of one or more alternative forms of a gene, all of which alleles relate to at least one trait or characteristic. In a diploid cell, the two alleles of a given gene occupy corresponding loci on a pair of homologous chromosomes.

As used herein, the term “locus” (loci plural) can mean any site at which an edit to the native genomic sequence is desired. In one embodiment, said term can mean a specific place or places or a site on a chromosome where for example a gene or genetic marker is found.

As used herein, the term “genetically linked” can refer to two or more traits that are co-inherited at a high rate during breeding such that they are difficult to separate through crossing.

A “recombination” or “recombination event” as used herein can refer to a chromosomal crossing over or independent assortment.

As used herein, the term “phenotype” can refer to the observable characteristics of an individual cell, cell culture, organism, or group of organisms, which results from the interaction between that individual's genetic makeup (i.e., genotype) and the environment.

As used herein, the term “chimeric” or “recombinant” when describing a nucleic acid sequence or a protein sequence can refer to a nucleic acid, or a protein sequence, that links at least two heterologous polynucleotides, or two heterologous polypeptides, into a single macromolecule, or that rearranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence can comprise at least one nucleotide difference when compared to any other naturally occurring nucleotide sequence.

As used herein, the term “nucleic acid” can refer to a polymeric form of nucleotides of any length, either ribonucleotides or deoxyribonucleotides, or analogs thereof. This term can refer to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” can refer to any segment of DNA associated with a biological function. Thus, genes can include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” or “orthologue” is known in the art and can refer to related sequences that share a common ancestor or family member and are determined based on the degree of sequence identity.

The terms “homology,” “homologous,” “substantially similar” and “corresponding substantially” can be used interchangeably herein. Said terms can refer to nucleic acid fragments wherein changes in one or more nucleotide bases do not affect the ability of the nucleic acid fragment to mediate gene expression or produce a certain phenotype. These terms can also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared.

“Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Sequence homology between amino acid or nucleic acid sequences can be defined in terms of shared ancestry. Two segments of nucleic acid can have shared ancestry because of either a speciation event (orthologs) or a duplication event (paralogs). Homology among amino acid or nucleic acid sequences can be inferred from their sequence similarity such that amino acid or nucleic acid sequences are said to be homologous is said amino acid or nucleic acid sequences share significant similarity. Significant similarity can be strong evidence that two sequences are related by divergent evolution from a common ancestor. Alignments of multiple sequences can be used to discover the homologous regions. Homology can be determined using software programs readily available in the art, such as those discussed in Current Protocols in Molecular Biology (F. M. Ausubel et al., eds., 1987) Supplement 30, section 7.718, Table 7.71. Some alignment programs are BLAST (NCBI), MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “endogenous” or “endogenous gene,” can refer to the naturally occurring gene, in the location in which it is naturally found within the host cell genome. In the context of the present disclosure, operably linking a heterologous promoter to an endogenous gene means genetically inserting a heterologous promoter sequence in front of an existing gene, in the location where that gene is naturally present. An endogenous gene as described herein can include alleles of naturally occurring genes that have been mutated according to any of the methods of the present disclosure.

As used herein, the term “exogenous” can be used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, the term “nucleotide change” refers to, e.g., nucleotide substitution, deletion, and/or insertion, as is well understood in the art. For example, mutations can contain alterations that produce silent substitutions, additions, or deletions, but do not alter the properties or activities of the encoded protein or how the proteins are made. Alternatively, mutations can be nonsynonymous substitutions or changes that can alter the amino acid sequence of the encoded protein and can result in an alteration in properties or activities of the protein.

As used herein, the term “protein modification” can refer to, e.g., amino acid substitution, amino acid modification, deletion, and/or insertion, as is well understood in the art.

As used herein, the term “at least a portion” or “fragment” of a nucleic acid or polypeptide can mean a portion having the minimal size characteristics of such sequences, or any larger fragment of the full-length molecule, up to and including the full-length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full-length polypeptide. The length of the portion to be used can depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides can also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri etal. (1997) Nature Biotech. 15:436-438; Moore etal. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

For PCR amplifications disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3^{rd} ed., Cold Spring Harbor Laboratory Press, Plainview, N.Y.). See also Innis et al., eds. (1990) PCR Protocols: A Guide to Methods and Applications (Academic Press, N.Y.); Innis and Gelfand, eds. (1995) PCR Strategies (Academic Press, N.Y.); and Innis and Gelfand, eds. (1999) PCR Methods Manual (Academic Press, N.Y.). Known methods of PCR include, but are not limited to, methods using paired primers, nested primers, single specific primers, degenerate primers, gene-specific primers, vector-specific primers, partially mismatched primers, and the like.

The term “primer” as used herein can refer to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer can be single stranded for maximum efficiency in amplification. The primer can be an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

As used herein, the term “forward primer” can refer to one of the two types of primers used in a PCR setup that anneals or hybridizes to the antisense or (−) strand of a double-stranded nucleic acid or DNA. The antisense strand can also be referred to as the “bottom strand” of a double-stranded nucleic acid or DNA.

As used herein, the term “reverse primer” can refer to one of the two types of primers used in a PCR setup that anneals or hybridizes to the sense or (+) strand of a double-stranded nucleic acid or DNA. The sense strand can also be referred to as the “top strand” of a double-stranded nucleic acid or DNA.

As used herein, the term “proximal end” can refer to the 5′ end of a single stranded nucleic acid (e.g., DNA) or the 5′ end of the top or sense strand of a double-stranded nucleic acid (e.g., DNA).

As used herein, the term “distal end” can refer to the 3′ end of a single-stranded nucleic acid (e.g., DNA) or the 3′ end of the top or sense strand of a double-stranded nucleic acid (e.g., DNA).

As used herein, the term “directed to” or “binds to” in the context of primers or assembly overlap sequences can refer to annealing or hybridizing between complementary sequences on separate nucleic acid fragments or polynucleotides.

As used herein, “promoter” can refer to a DNA sequence capable of controlling the expression of a coding sequence or functional RNA. In some embodiments, the promoter sequence consists of proximal and more distal upstream elements, the latter elements often referred to as enhancers. Accordingly, an “enhancer” can be a DNA sequence that can stimulate promoter activity and may be an innate element of the promoter or a heterologous element inserted to enhance the level or tissue specificity of a promoter. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, the phrases “recombinant construct”, “expression construct”, “chimeric construct”, “construct”, and “recombinant DNA construct” are used interchangeably herein. A recombinant construct can comprise an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source but arranged in a manner different than that found in nature. Such construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by direct sequencing, Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

“Operably linked” or “functionally linked” can mean the sequential arrangement of any functional payload according to the disclosure (e.g., promoter, terminator, degron, solubility tag, etc.) with a further oligo- or polynucleotide. In some cases, the sequential arrangement can result in transcription of said further polynucleotide. In some cases, the sequential arrangement can result in translation of said further polynucleotide. The functional payloads can be present upstream or downstream of the further oligo or polynucleotide. In one example, “operably linked” or “functionally linked” can mean a promoter controls the transcription of the gene adjacent or downstream or 3′ to said promoter. In another example, “operably linked” or “functionally linked” can mean a terminator controls termination of transcription of the gene adjacent or upstream or 5′ to said terminator.

The term “product of interest” or “biomolecule” as used herein can refer to any product produced by microbes from feedstock. In some cases, the product of interest may be a small molecule, enzyme, peptide, amino acid, organic acid, synthetic compound, fuel, alcohol, etc. For example, the product of interest or biomolecule may be any primary or secondary extracellular metabolite. The primary metabolite may be, inter alia, ethanol, citric acid, lactic acid, glutamic acid, glutamate, lysine, threonine, tryptophan and other amino acids, vitamins, polysaccharides, etc. The secondary metabolite may be, inter alia, an antibiotic compound like penicillin, or an immunosuppressant like cyclosporin A, a plant hormone like gibberellin, a statin drug like lovastatin, a fungicide like griseofulvin, etc. The product of interest or biomolecule may also be any intracellular component produced by a microbe, such as: a microbial enzyme, including catalase, amylase, protease, pectinase, glucose isomerase, cellulase, hemicellulase, lipase, lactase, streptokinase, and many others. The intracellular component may also include recombinant proteins, such as insulin, hepatitis B vaccine, interferon, granulocyte colony-stimulating factor, streptokinase and others.

As used herein, the term “HTP genetic design library” or “library” refers to collections of genetic perturbations according to the present disclosure. In some embodiments, the libraries of the present disclosure may manifest as (i) a collection of sequence information in a database or other computer file, (ii) a collection of genetic constructs encoding for the aforementioned series of genetic elements, or (iii) host cell strains comprising said genetic elements. In some embodiments, the libraries of the present disclosure may refer to collections of individual elements (e.g., collections of promoters for PRO swap libraries, collections of terminators for STOP swap libraries, collections of protein solubility tags for SOLUBILITY TAG swap libraries, or collections of protein degradation tags for DEGRADATION TAG swap libraries). In other embodiments, the libraries of the present disclosure may also refer to combinations of genetic elements, such as combinations of promoter:genes, gene:terminator, or even promoter:gene:terminators. In some embodiments, the libraries of the present disclosure may also refer to combinations of promoters, terminators, protein solubility tags and/or protein degradation tags. In some embodiments, the libraries of the present disclosure further comprise meta data associated with the effects of applying each member of the library in host organisms. For example, a library as used herein can include a collection of promoter: :gene sequence combinations, together with the resulting effect of those combinations on one or more phenotypes in a particular species, thus improving the future predictive value of using said combination in future promoter swaps.

As used herein, the term “SNP” refers to Small Nuclear Polymorphism(s). In some embodiments, SNPs of the present disclosure should be construed broadly, and include single nucleotide polymorphisms, sequence insertions, deletions, inversions, and other sequence replacements. As used herein, the term “non-synonymous” or non-synonymous SNPs” refers to mutations that lead to coding changes in host cell proteins

A “high-throughput (HTP)” method of genomic engineering may involve the utilization of at least one piece of automated equipment (e.g. a liquid handler or plate handler machine) to carry out at least one-step of said method.

The term “polynucleotide” as used herein encompasses oligonucleotides and refers to a nucleic acid of any length. Polynucleotides may be DNA or RNA. Polynucleotides may be single-stranded (ss) or double-stranded (ds) unless otherwise specified. Polynucleotides may be synthetic, for example, synthesized in a DNA synthesizer, or naturally occurring, for example, extracted from a natural source, or derived from cloned or amplified material. Polynucleotides referred to herein can contain modified bases or nucleotides.

The term “pool”, as used herein, can refer to a collection of at least 2 polynucleotides. In some embodiments, a set of polynucleotides may comprise at least 5, at least 10, at least 12 or at least 15 or more polynucleotides. In some embodiments, a set of polynucleotides may comprise at least 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000, 2000, 3000, 4000, 5000, 6000, 7000, 8000, 9000, 10000 or more polynucleotides.

The term “overlapping sequence”, or “overlapping assembly sequence” or “assembly overlap sequence” as used herein can refer to a sequence that is complementary in two polynucleotides and where the overlapping sequence is ss, on one polynucleotide such that it can be hybridized to another overlapping complementary ss region on another polynucleotide. An overlapping sequence may be at or close to (e.g., within about 5, 10, 20 nucleotides of) the terminal ends of two distinct polynucleotides. For example, if the two distinct polynucleotides are single-stranded, then the assembly overlap sequence can be present on the 5′ and 3′ terminal ends of each of the single-stranded (ss) polynucleotides with the sequences being in a reverse complementary orientation on one of the ss polynucleotides relative to the other ss polynucleotide. Alternatively, if the two distinct polynucleotides are double-stranded, then the assembly overlap sequence of one of the polynucleotides can be present on the 3′ terminal end of said polynucleotide (i.e., 3′ end in reference to the top strand of the ds polynucleotide), while the complementary assembly overlap sequence on the other polynucleotide can be present at the 5′ end of said polynucleotide (i.e., 5′ end in reference to the top strand of the ds polynucleotide). As necessary, the assembly overlap sequence on any double-stranded (ds) polynucleotide may be made available by removing any non-overlapping sequence. The removal can be enzymatic such as through the use of a 3′-5′ exonuclease activity of a polymerase or other exonucleases (e.g., T5, etc.).

As used herein, the term “assembling”, can refer to a reaction in which two or more, four or more, six or more, eight or more, ten or more, 12 or more 15 or more polynucleotides, e.g., four or more polynucleotides are joined to another to make a longer polynucleotide.

As used herein, the term “incubating under suitable reaction conditions”, can refer to maintaining a reaction at a suitable temperature and time to achieve the desired results, i.e., polynucleotide assembly. Reaction conditions suitable for the enzymes and reagents used in the present method are known (e.g. as described in the Examples herein) and, as such, suitable reaction conditions for the present method can be readily determined. These reactions conditions may change depending on the enzymes used (e.g., depending on their optimum temperatures, etc.).

As used herein, the term “joining”, can refer to the production of covalent linkage between two sequences.

As used herein, the term “composition” can refer to a combination of reagents that may contain other reagents, e.g., glycerol, salt, dNTPs, etc., in addition to those listed. A composition may be in any form, e.g., aqueous or lyophilized, and may be at any state (e.g., frozen or in liquid form).

As used herein a “vector” is a suitable DNA into which a fragment or DNA assembly may be integrated such that the engineered vector can be replicated in a host cell. A linearized vector may be created restriction endonuclease digestion of a circular vector or by PCR. The concentration of fragments and/or linearized vectors can be determined by gel electrophoresis or other means.

### Overview

Provided herein are compositions and methods utilizing said compositions to facilitate the rapid and cost-effective generation of linear nucleic acid (e.g., DNA) sequences suitable for homology-directed editing of genetic element(s) within a desired or target cell. The linear nucleic acid sequences generated using the methods and compositions provided herein can be used directly for editing genetic elements within a host cell without requiring further cloning or assembly methods to make the linear nucleic acid sequences suitable for editing. Some of the methods provided herein use compositions comprising pooled oligonucleotides to amplify a nucleic acid template that comprises at least one genetic edit (also referred to as a payload sequence). The amplification can facilitate appendage of genomic targeting sequences (i.e., homology arms) to the nucleic acid template comprising the at least one genetic edit. Some of the methods provided herein use compositions comprising pooled oligonucleotides in an overlap assembly-based method to append genomic targeting sequences (i.e., homology arms) to opposing ends of a nucleic acid template that comprises at least one genetic edit (also referred to as a payload sequence). The overlap assembly methods can be any overlap assembly known in the art, such as, for example, Golden Gate Assembly®, Gibson Assembly® or HiFi Assembly®. Due to the pooled nature of the oligonucleotides used in the compositions and methods provided herein, the output of the assembly methods provided herein can be a pool of nucleic acid sequences that can direct the at least one genetic edit to one or a plurality of desired loci within a genetic element or elements within a target cell. As further described herein, enzymatic steps within the assembly methods provided herein can circularize the pool of nucleic acid sequences as well as linearize the pool at a different location within the circularized molecules to form a final pool of integration fragments (“circular permutation”). Specific steps within any of the methods provided herein can be utilized to amplify specific or select species of integration fragments from the final pool. Moreover, the methods and compositions provided herein can be used to generate libraries of integration fragments that can be suitable for any number of applications such as, for example, any genome editing methods or any pooled pathway assembly.

Provided herein is a composition comprising a mixture of polynucleotides for assembly into a library of nucleic acid constructs. The mixture can comprise n pools of targeting polynucleotides. Then pools can be at most, at least, or exactly 2, 3, 4, 5, 6, 7, 8, 9, 10, 11, 12, 13, 14, 15, 16, 17, 18, 19 or 20 pools of targeting polynucleotides. The mixture can further comprise n pools of insert or bridging polynucleotides. The n pools can be at most, at least, or exactly 2, 3, 4, 5, 6, 7, 8, 9, 10, 11, 12, 13, 14, 15, 16, 17, 18, 19 or 20 pools of insert or bridging polynucleotides. The insert or bridging polynucleotides within a pool can each comprise one or more payloads as described herein. Each of the pools of targeting polynucleotides can comprise sequence that binds to a distal or 3′ end of one of the n pools of insert or bridging polynucleotides at its 5′ end and sequence that binds to a proximal or 5′ end of the same one of the n pools of insert or bridging polynucleotides at its 3′ end. The insert polynucleotides can be designed such that the assembly results in a library of integration fragments where each integration fragment comprises homology arms from one of the n pools of targeting polynucleotides interspersed with a specific element or payload or genetic edit from one of the n pools of insert polynucleotides. The targeting polynucleotides in each of the n pools of targeting polynucleotides can comprise first and second homology arms that target a different locus in the genome of a host cell than the homology arms in each other targeting polynucleotide within a pool or between pools. The targeting polynucleotides in each of the n pools of targeting polynucleotides can comprise first and second homology arms that target a same locus in the genome of a host cell than the homology arms in each other targeting polynucleotide within a pool or between pools. For each of the n pools of targeting polynucleotides, said n pools can comprise a subset within said n pool that comprises targeting polynucleotides that comprise first and second homology arms that target a same locus in the genome of a host cell than the first and second homology arms in each other of the n pools of targeting polynucleotides. For each of then pools of targeting polynucleotides, said n pools can comprise a subset within said n pool that comprises targeting polynucleotides that comprise first and second homology arms that target a different locus in the genome of a host cell than the first and second homology arms in each other of the n pools of targeting polynucleotides.

In one aspect, provided herein is a composition comprising a pool of insert polynucleotides, and a pool of targeting polynucleotides, wherein each insert polynucleotide in the pool of insert polynucleotides comprises one or more payload sequences, wherein, for each insert polynucleotide, the composition comprises one or a plurality of targeting polynucleotide(s) from the pool of targeting polynucleotides, wherein the one or each of the plurality of targeting polynucleotide(s) comprises from 5′ to 3′, a first assembly overlap sequence that binds to a distal or 3′ end of the insert polynucleotide, a first homology arm, a linearization sequence, a second homology arm and a second assembly overlap sequence comprising sequence that binds to a proximal or 5′ end of the insert polynucleotide, wherein the first homology arm and the second homology arm comprise sequence complementary to a genomic locus in a host cell. The first assembly overlap sequence can bind to the distal end of the insert polynucleotide via sequence in the first assembly overlap sequence that is complementary to the distal end of the insert polynucleotide. The second assembly overlap sequence can bind to the proximal end of the insert polynucleotide via sequence in the second assembly overlap sequence that is complementary to the reverse complement of the proximal end of the insert polynucleotide. In other words, the second assembly overlap sequence can comprise sequence that is identical to or the same as the proximal end of the insert polynucleotide or a portion thereof. In some cases, the plurality of targeting polynucleotides comprises each of the targeting polynucleotides in the pool of targeting polynucleotides. In some cases, the plurality of targeting polynucleotides comprises a subset of the targeting polynucleotides in the pool of targeting polynucleotides. Each of the targeting polynucleotides in the pool of targeting polynucleotides can comprise first and second homology arms that target a different locus in the genome of a host cell than each other targeting polynucleotide in the pool. Each of the targeting polynucleotides in the pool of targeting polynucleotides can comprise first and second homology arms that target a same locus in the genome of a host cell. The pool of targeting polynucleotides can comprise a subset of targeting polynucleotides that comprise first and second homology arms that target a same locus in the genome of a host cell than the first and second homology arms in each other targeting polynucleotide in the pool of targeting polynucleotides. The pool of targeting polynucleotides can comprise a subset of targeting polynucleotides that comprise first and second homology arms that target a different locus in the genome of a host cell than the first and second homology arms in each other targeting polynucleotide in the pool of targeting polynucleotides.

Provided herein is a composition comprising a mixture of polynucleotides for assembly in a deterministic fashion of a library of nucleic acid constructs. The mixture can comprise n pools of targeting polynucleotides that serve as forward primers and reverse primers. Then pools can be at most, at least, or exactly 2, 3, 4, 5, 6, 7, 8, 9, 10, 11, 12, 13, 14, 15, 16, 17, 18, 19 or 20 pools of targeting polynucleotides and reverse primers. The n pools can each comprise an equal number of targeting polynucleotides and reverse primers or they can comprise differing numbers of targeting polynucleotides and reverse primers. In one embodiment, the mixture comprises 2 pools such that one of the two pools comprise targeting polynucleotides and the other of the two pools comprises reverse primers. Each pool of targeting polynucleotides can comprise a paired reverse primer in a separate pool of reverse primers. The mixture can further comprise n-1 pools of insert or bridging polynucleotides. Each targeting polynucleotide can comprise sequence that binds (e.g., sequence that is complementary to) a distal or 3′ end of one of the n-1 pools of insert or bridging polynucleotides at its 5′ end and sequence that binds to (e.g., sequence that is complementary to a reverse complement of) a proximal or 5′ end of the same one of the n-1 pools of insert or bridging polynucleotides at its 3′ end. Each of the insert or bridging polynucleotides in an n-1 pool can comprise one or more payloads as described herein. Each reverse primer can comprise sequence that binds (e.g., via sequence that is complementary to) a distal or 3′ end of one of the n-1 pools of insert or bridging polynucleotides. The insert polynucleotides can be designed such that the assembly results in a library of integration fragments where each integration fragment comprises homology arms from one of the n pools of targeting polynucleotides interspersed with a specific element (e.g., payload or genetic edit) from one of the n-1 pools of insert polynucleotides. The targeting polynucleotides in each of then pools of targeting polynucleotides can comprise first and second homology arms that target a different locus in the genome of a host cell than the homology arms in each other targeting polynucleotide within a pool or between pools. The targeting polynucleotides in each of the n pools of targeting polynucleotides can comprise first and second homology arms that target a same locus in the genome of a host cell than the homology arms in each other targeting polynucleotide within a pool or between pools. For each of the n pools of targeting polynucleotides, said n pools can comprise a subset within said n pool that comprises targeting polynucleotides that comprise first and second homology arms that target a same locus in the genome of a host cell than the first and second homology arms in each other of the n pools of targeting polynucleotides. For each of then pools of targeting polynucleotides, said n pools can comprise a subset within said n pool that comprises targeting polynucleotides that comprise first and second homology arms that target a different locus in the genome of a host cell than the first and second homology arms in each other of the n pools of targeting polynucleotides.

In another aspect, provided herein is a composition comprising a pool of insert polynucleotides, a pool of targeting polynucleotides which serve as forward primers, and a pool of reverse primers, wherein each insert polynucleotide in the pool of insert polynucleotides comprises one or more payload sequences, wherein, for each insert polynucleotide, the composition comprises one or a plurality of targeting polynucleotide(s) from the pool of targeting polynucleotides, wherein the one or plurality of targeting polynucleotide(s) comprises from 5′ to 3′, a first assembly overlap sequence comprising sequence that binds to a distal or 3′ end of the insert polynucleotide, a first homology arm, a linearization sequence, a second homology arm and a second assembly overlap sequence that binds to a proximal or 5′ end of the insert polynucleotide, and wherein the reverse primer comprises sequence that binds to the distal or 3′ end of the insert polynucleotide. The first assembly overlap sequence and/or reverse primer can bind to the distal end of the insert polynucleotide via sequence in the first assembly overlap sequence and/or reverse primer that is complementary to the distal end of the insert polynucleotide. The second assembly overlap sequence can bind to the proximal end of the insert polynucleotide via sequence in the second assembly overlap sequence that is complementary to the reverse complement of the proximal end of the insert polynucleotide. In other words, the second assembly overlap sequence can comprise sequence that is identical to or the same as the proximal end of the insert polynucleotide or a portion thereof. The first homology arm and the second homology arm in each targeting polynucleotide can comprise sequence complementary to a genomic locus in a host cell. In some cases, the plurality of targeting polynucleotides comprises each of the targeting polynucleotides in the pool of targeting polynucleotides. In some cases, the plurality of targeting polynucleotides comprises a subset of the targeting polynucleotides in the pool of targeting polynucleotides. Each of the targeting polynucleotides in the pool of targeting polynucleotides can comprise first and second homology arms that target a different locus in the genome of a host cell than each other targeting polynucleotide in the pool. Each of the targeting polynucleotides in the pool of targeting polynucleotides can comprise first and second homology arms that target a same locus in the genome of a host cell. The pool of targeting polynucleotides can comprise a subset of targeting polynucleotides that comprise first and second homology arms that target a same locus in the genome of a host cell than the first and second homology arms in each other targeting polynucleotide in the pool of targeting polynucleotides. The pool of targeting polynucleotides can comprise a subset of targeting polynucleotides that comprise first and second homology arms that target a different locus in the genome of a host cell than the first and second homology arms in each other targeting polynucleotide in the pool of targeting polynucleotides. Each targeting polynucleotide in the pool of targeting polynucleotides can be paired with a revers primer from the pool of reverse primers. The insert polynucleotides can be double-stranded, and the terms proximal end and distal end can be in reference to the top or sense strand.

In one embodiment, provided herein is a method for generating libraries of polynucleotides, the method comprising: (a) combining n pools of polynucleotide parts (e.g., targeting polynucleotides and reverse primers) and n-1 pools of insert or bridging polynucleotides; and (b) assembling the n pools of polynucleotide parts and n-1 pools of insert polynucleotides into a library of polynucleotides, wherein each polynucleotide in the library comprises a defined combination of an individual element from each of the n pools of polynucleotide parts and insert polynucleotides. Each targeting polynucleotide can comprise sequence complementary to a distal or 3′ end of one of the n-1 pools of insert or bridging polynucleotides at its 5′ end and sequence complementary to a proximal or 5′ end of the same one of the n-1 pools of insert or bridging polynucleotides at its 3′ end. In another embodiment, provided herein is a method for generating libraries of polynucleotides, the method comprising: (a) combining n pools of targeting polynucleotides and n pools of insert or bridging polynucleotides; and (b) assembling the n pools of targeting polynucleotides and n pools of insert polynucleotides into a library of polynucleotides, wherein each polynucleotide in the library comprises a defined combination of an individual element from each of the n pools of targeting polynucleotides and insert polynucleotides. Each targeting polynucleotide can comprise sequence complementary to a distal or 3′ end of one of the n pools of insert or bridging polynucleotides at its 5′ end and sequence complementary to a proximal or 5′ end of the same one of the n pools of insert or bridging polynucleotides at its 3′ end. The assembling can be performed via an in vitro overlap assembly method. In some cases, the assembling is performed via an in vitro cloning method, wherein the mixture of the n pools of polynucleotide parts (i.e., n pools of targeting polynucleotides and n pools of insert or bridging polynucleotides) and/or n-1 pools of insert or bridging polynucleotides is heated to partially or fully denature any double-stranded polynucleotide parts present, then cooled at a slow rate to room temperature before being subjected to the in vitro cloning method.

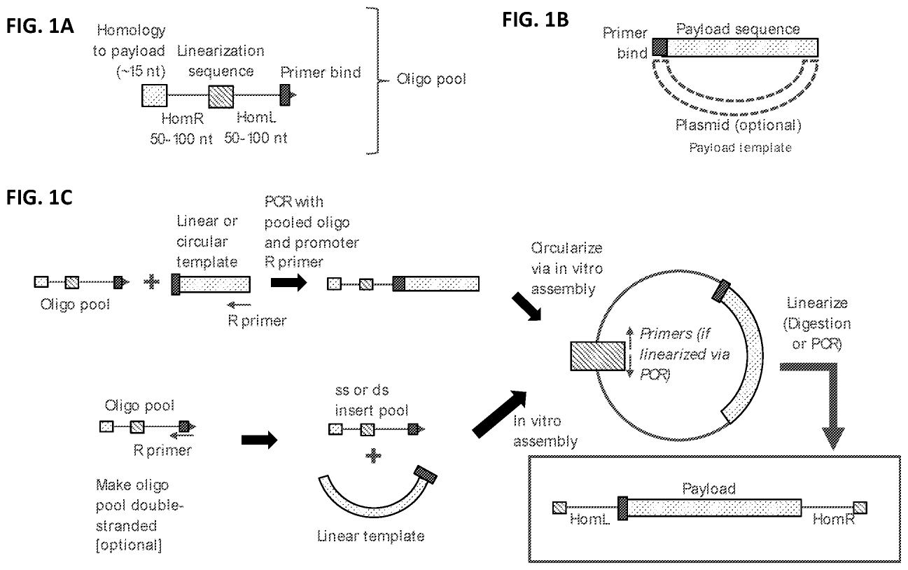

In one embodiment, provided herein is a method for genetically editing a host cell, the method comprising: (a) assembling a pool of insert polynucleotides and a pool of targeting polynucleotides into a pool of circular molecules, wherein each circular molecule from the pool of circular molecules comprises one or more payload sequences flanked by a first homology arm 5′ to the one or more payload sequences and a second homology arm 3′ to the one or more payload sequences and a linearization sequence that is located between both the first and second homology arms; (b) linearizing each of the circular molecules from the pool of circular molecules via the linearization sequence present on each circular molecule, thereby generating a pool of linear insert polynucleotides, wherein each linear insert polynucleotide in the pool comprises from 5′ to 3′ a first homology arm, one or more payload sequences and a second homology arm, wherein the first homology arm and the second homology arm comprise sequence complementary to a genomic locus in a host cell; and (c) introducing the pool of linear insert polynucleotides into the host cell, thereby genetically editing the host cell. An exemplary schematic of steps (a) and (b) of this editing method are depicted in FIGS. 1A-1C.

In one embodiment, the assembling of step (a) comprises: (i) generating a mixture for performing a polymerase chain reaction (PCR), wherein the pool of insert polynucleotides serves as template, the pool of targeting polynucleotides serves as forward primers and providing a pool of reverse primers; (ii) performing PCR on the mixture; and (iii) circularizing the amplicons generated in step (a)(ii) using a nucleic acid assembly method. For each insert polynucleotide in step (a)(i), the mixture can comprise a forward primer or a plurality of forward primers from the pool of forward primers and a reverse primer from the pool of reverse primers. The forward primer or each of the plurality of forward primers can comprise, from 5′ to 3′, a first assembly overlap sequence comprising sequence that binds to a distal end of the insert polynucleotide, the first homology arm, the linearization sequence, the second homology arm and a second assembly overlap sequence comprising sequence that binds to a proximal end of the insert polynucleotide. The reverse primer can comprise sequence that binds to the distal end of the insert polynucleotide. For each insert polynucleotide in step (a)(ii), the PCR can generate a PCR product comprising, from 5′ to 3′, the first assembly overlap sequence, the first homology arm, the linearization sequence, the second homology arm and the one or more payload sequences. The assembly method of step (a)(iii) for circularizing of the PCR products from step (a)(ii) can be any known nucleic acid assembly method known in the art. In one embodiment, the assembly method is selected from the group consisting of splicing and overlap-extension PCR (SOE-PCR), Uracil-specific excision reagent (USER) cloning, restriction-ligation, scarless restriction-ligation, blunt-end ligation, overlap based assembly method and recombination-based method, or any other enzymatic or chemical method of joining two DNA molecules. The first assembly overlap sequence of any targeting polynucleotide and/or reverse primer can bind to the distal end of the insert polynucleotide via sequence in the first assembly overlap sequence and/or reverse primer that is complementary to the distal or 3′ end of the insert polynucleotide. The second assembly overlap sequence of any targeting polynucleotide can bind to the proximal end of the insert polynucleotide via sequence in the second assembly overlap sequence that is complementary to the reverse complement of the proximal or 5′ end of the insert polynucleotide. In other words, the second assembly overlap sequence can comprise sequence that is identical to or the same as the proximal end of the insert polynucleotide or a portion thereof. The insert polynucleotides can be double-stranded, and the terms proximal end and distal end can be in reference to the top or sense strand. An exemplary schematic of this embodiment is depicted in the upper portion of FIG. 1C.

In another embodiment, the assembling of step (a) comprises directly performing an assembly method on a mixture comprising the pool of insert polynucleotides and the pool of targeting polynucleotides. Further to this embodiment, for each insert polynucleotide, the mixture can comprise a targeting polynucleotide a plurality of targeting polynucleotides from the pool of targeting polynucleotides. The targeting polynucleotide or each of the plurality of targeting polynucleotides can comprise, from 5′ to 3′, a first assembly overlap sequence comprising sequence that binds to a distal end of the insert polynucleotide, the first homology arm, the linearization sequence, the second homology arm and a second assembly overlap sequence comprising sequence that binds to a proximal end of the insert polynucleotide. The targeting polynucleotide(s) can be a single-stranded (ss) or double-stranded (ds) polynucleotide. The insert polynucleotides can be ds and the terms proximal end and distal end can be in reference to the top or sense strand. In one embodiment, the targeting polynucleotide or each targeting polynucleotide from the plurality is single-stranded (ss) and is converted to a double-stranded (ds) polynucleotide prior to being subjected to the assembly method. As shown in FIG. 1C, conversion of the ss targeting polynucleotide can be accomplished by mixing the targeting polynucleotide with a primer comprising sequence that binds to the 3′ end of the targeting polynucleotide and performing a primer extension reaction with a suitable polymerase. The assembly method can be any known nucleic acid assembly method known in the art. In one embodiment, the assembly method is selected from selected from the group consisting of splicing and overlap-extension PCR (SOE-PCR), Uracil-specific excision reagent (USER) cloning, restriction-ligation, blunt-end ligation, overlap based assembly method and recombination-based method, or any other enzymatic or chemical method of joining two DNA molecules. An exemplary schematic of this embodiment is depicted in the lower portion of FIG. 1C.

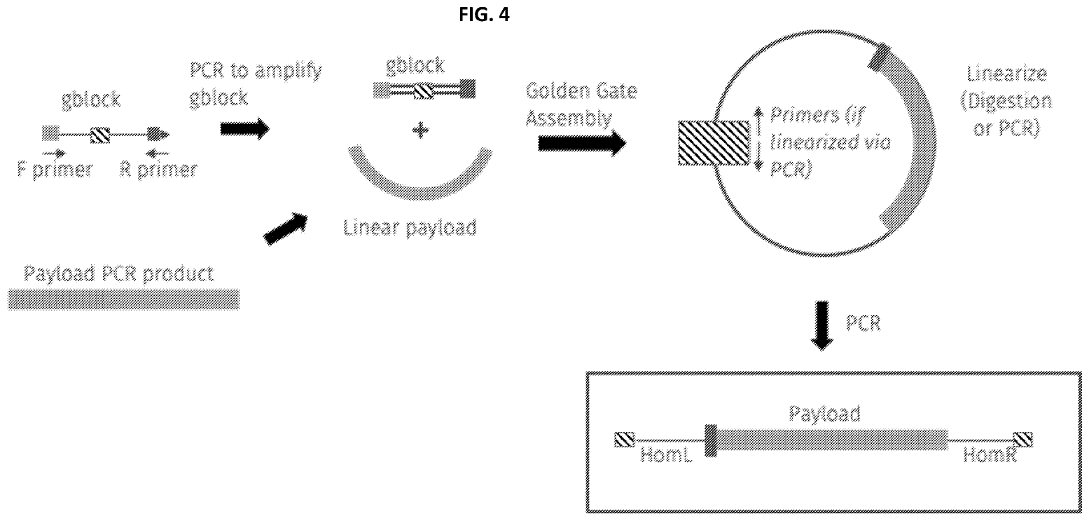

In yet another embodiment, the assembling of step (a) comprises directly performing an overlap assembly method employing a Type IIS restriction enzyme and ligase (i.e., Golden Gate Assembly®) on a mixture comprising the pool of insert polynucleotides and the pool of targeting polynucleotides. An exemplary schematic of this assembling method is depicted in FIG. 4. In order to perform this assembly method, Type IIS restriction enzyme sites for a particular Type IIS restriction enzyme must be present on opposing ends of the targeting polynucleotides or subsets thereof in the pool of targeting polynucleotides and the opposing ends of the insert polynucleotides or subsets thereof in the pool of polynucleotides. Digestion of the Type IIS restriction enzyme sites with the appropriate Type IIS restriction enzyme during Golden Gate Assembly® can subsequently generate overhangs in the targeting polynucleotides or subsets thereof that comprise the Type IIS restriction sites that are compatible with the overhangs generated in the insert polynucleotides or subsets thereof that comprise the Type IIS restriction sites. The targeting polynucleotides and/or insert polynucleotides can be synthesized with the Type IIS restriction enzyme sites present on the opposing ends or the Type IIS restriction enzymes sites can be appended to the opposing ends of the target polynucleotides or insert polynucleotides. The Type IIS restriction enzyme sites can be appended to opposing ends of the targeting polynucleotides and/or insert polynucleotides via PCR using primer pairs comprising sequence that binds to the ends of the targeting polynucleotides or insert polynucleotides and non-complementary sequencing containing tails that comprise the Type IIS restriction enzyme sites. For example, the tails on each primer in the primer pair can comprise, from 5′ to 3′, random sequence, a recognition sequence for the Type IIS restriction enzyme and a site that allows for ligation onto an intended targeting polynucleotide and/or insert polynucleotide. The matching Type IIS restriction enzyme sites on the opposing ends of the target polynucleotides and insert polynucleotides can be for any Type IIS restriction enzyme known in the art. In one embodiment, the Type IIS restriction enzyme sites can be for Type IIS restriction enzymes that create 4-base overhangs such as, for example, Bsal, Bbsl, BsmBI and Esp3I, in order to facilitate the ordered assembly of targeting polynucleotides and insert polynucleotides or subsets thereof.

Further to the above embodiments, the circularized molecules of step (a) can be amplified prior to step (b). In some cases, the linearizing of step (b) comprises rolling circle amplification (RCA) of each circular molecule from the pool of circular molecules, wherein the RCA of each circular molecule produces a concatenated linear product comprising repeated units each separated by the linearization sequence. Each of the repeated units comprises the insert polynucleotide flanked upstream by the first homology arm and downstream by the second homology arm. The insert polynucleotides with appended first and second homology arms can be released from the concatenated linear product via the linearization sequence present between each repeated unit, thereby generating the pool of linear insert polynucleotides. Releasing from the concatenated linear product via the linearization sequence between each repeated unit can be via PCR using primers pair directed against the linearization sequence as provided herein or via digestion of a recognition sequence of a restriction enzyme (e.g., Type IIS restriction enzyme) within the linearization sequence as provided herein.

In one embodiment, the introducing in step (c) entails performing double-crossover integration of the pool of linear insert polynucleotides in the host cell. In another embodiment, the introducing in step (c) entails performing CRISPR-mediated homology directed repair with the pool of linear insert polynucleotides in the host cell. In yet another embodiment, the introducing in step (c) entails performing lambda red mediated integration of the pool of linear insert polynucleotides in the host cell.

In embodiments where CRISPR-mediated homology directed repair is performed, the method further comprises introducing a pool of guide RNAs (gRNAs) into the host cell. The pool of gRNAs can be introduced into the host cell prior to, along with or following the introduction of the pool of linear insert polynucleotides. Each of the gRNAs in the pool of gRNAs can comprise sequence complementary to a genomic locus targeted by the homology arms in one or more of the linear insert polynucleotides present in the pool of linear insert polynucleotides. Each of the gRNAs in the pool of gRNAs can comprise sequence complementary to a genomic locus targeted by the homology arms in one or more of the targeting polynucleotides. The pool of gRNAs can comprise gRNAs that target or bind the genomic loci targeted by each of the linear insert polynucleotides in the pool of linear insert polynucleotides. The pool of gRNAs can comprise gRNAs that target or bind the genomic loci targeted by each of the targeting polynucleotides in the pool of targeting polynucleotides. The pool of gRNAs can comprise gRNAs that target or bind genomic loci targeted by a subset of linear insert polynucleotides in the pool of linear insert polynucleotides. The pool of gRNAs can comprise gRNAs that target or bind genomic loci targeted by a subset of targeting polynucleotides in the pool of targeting polynucleotides. In embodiments where CRISPR-mediated homology directed repair is performed, each of the linear insert polynucleotides in the pool of linear insert polynucleotides serve as donor nucleic acid fragments as described herein.

Further to the above embodiments, the insert polynucleotides can comprise one or more payload sequence as provided herein. The insert polynucleotides can be a synthetic DNA fragment, a PCR product, or other single- or double-stranded DNA fragment. The pool of targeting polynucleotides and/or reverse primers can be synthesized using array-based or column-based synthetic methods known in the art. In one embodiment, each of the targeting polynucleotides can be gBlocks®. In one embodiment, each of the insert polynucleotides can be gBlocks®.

### Targeting Polynucleotides

As described herein, the compositions and methods provided herein can comprise or utilize targeting polynucleotides that comprise homology arms that target a specific genomic locus. Each targeting polynucleotide can comprise, from 5′ to 3′, a first assembly overlap sequence comprising sequence that binds to a distal or 3′ end of an insert polynucleotide, a first homology arm, a linearization sequence, a second homology arm and a second assembly overlap sequence comprising sequence that binds to a proximal or 5′ end of the same insert polynucleotide as the first assembly overlap sequence. The first and second homology arms can comprise sequence complementary to sequence present at a target locus in a genetic element (e.g., cosmid, plasmid, chromosome) within a host cell. The linearization sequence present in each targeting polynucleotide can be used to generate a linear fragment for any circularized molecule generated following assembly of targeting polynucleotide with an insert polynucleotide using a method provided herein. The linearization sequence present in each targeting polynucleotide can be used to generate a linear fragment for any concatenated product generated following RCA of a circularized molecule generated following assembly of targeting polynucleotide with an insert polynucleotide using a method provided herein. The targeting polynucleotides can be chemically synthesized (e.g., array-synthesized or column-synthesized) using any of the methods known in the art for synthesizing nucleic acids. The targeting polynucleotides can be gBlocks® (see, for example, FIG. 4). The targeting polynucleotides can be amplified via an extension reaction (e.g., PCR) from existing DNA such as, for example, genomic DNA. In embodiments in which a targeting polynucleotide and an insert polynucleotide are assembled using PCR, the targeting polynucleotide can be a forward primer and can be paired with a reverse primer as provided herein. The targeting polynucleotides can further comprise additional elements such as, for example, barcodes and gene coding sequence modifications or portions thereof. The barcodes can comprise a sequence unique to the first and second homology arms on a particular targeting polynucleotide flanked by sequence universal to the barcode sequence present in each other targeting polynucleotide. The sequence universal to the barcode sequence present in each other targeting polynucleotide can then be used for amplifying or sequencing the unique sequence in each barcode. The additional elements can flank one or both of the homology arms. The targeting polynucleotides for use in any of the methods provided herein can be single-stranded or double-stranded. Single-stranded targeting polynucleotides can be made double-stranded prior to use in any of the methods provided herein using any method known in the art and/or provided herein.

A targeting polynucleotide for use in a composition, kit or method provided herein can vary in length and, in some cases, can be at least 1, 2, 3, 4, 5, 6, 7, 8, 9, 10, 11 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 200, 300, 400, 500, 600, 700, 800, 900, 950 or 1000 nucleotide bases in length and/or may be more than 1 kb or 2 kb in length. Alternatively, a targeting polynucleotide can be 2 kb or more, or 1 kb or more or more than 900 bases, 800 bases, 700 bases, 600 bases, 500 bases, 400 bases, 300 bases, 200 bases or 100 bases in length. The targeting polynucleotide length can be in the range of 100 nucleotides-2 kb for example up to 100, up to 150, up to 200, up to 250, up to 300, up to 350, up to 400, up to 450, up to 500, up to 550, up to 600, up to 650, up to 700, up to 750, or up to 800, up to 850, up to 900, up to 950, up to 1000, up to 1500, or up to 2000 nucleotides. The minimum length of a targeting polynucleotide may be defined by a preferable Tm that is determined empirically.

As described herein, each of the targeting polynucleotides can comprise a pair of homology arms such that each member of the pair of homology arms, which can be referred to as first and second homology arms, comprises sequence complementary to sequence present at a desired or target locus in a genetic element (e.g., cosmid, plasmid, chromosome, etc.) in a host cell. The first and/or second homology arms can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more nucleotides in length. The first and/or the second homology arms can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 nucleotides in length. The first assembly overlap and/or the second assembly overlap sequence can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 nucleotides in length. The length of the first and/or second homology arms can be in the range of 15 nucleotides-100 nucleotides for example up to 20, up to 25, up to 30, up to 35, up to 40, up to 45, up to 50, up to 55, up to 60, up to 65, up to 70, up to 75, up to 80 nucleotides, up to 85 nucleotides, up to 90 nucleotides, up to 95 nucleotides or up to 100 nucleotides in length. In one embodiment, the first and second homology arms on each targeting polynucleotide in a pool of targeting polynucleotides can comprise sequence complementary to a different genomic locus in a host cell as compared to each other first and second homology arms on each other targeting polynucleotide in the pool. In another embodiment, the first and second homology arms on each targeting polynucleotide in a pool of targeting polynucleotides can comprise sequence complementary to an identical or the same genomic locus in a host cell as compared to each other first and second homology arms on each other targeting polynucleotide in the pool. In yet another embodiment, the first and second homology arms on a subset of targeting polynucleotide in a pool of targeting polynucleotides can comprise sequence complementary to an identical or the same genomic locus in a host cell as compared to each other first and second homology arms on each other targeting polynucleotide in the pool. In still another embodiment, the first and second homology arms on a subset of targeting polynucleotide in a pool of targeting polynucleotides can comprise sequence complementary to a different genomic locus in a host cell as compared to each other first and second homology arms on each other targeting polynucleotide in the pool.

As described herein, each of the targeting polynucleotides can comprise sequence that aids in the assembly of said targeting polynucleotides with an insert polynucleotide, which can be referred to as assembly overlap sequences. In order to aid in said assembly, said assembly overlap sequences can be complementary to the sequences or reverse complements thereof present on insert polynucleotides. As described herein, the first assembly overlap sequence can comprise sequence that binds to a distal portion of an insert polynucleotide, while the second assembly overlap sequence can comprise sequence binds to a proximal portion of the same insert polynucleotide. The first assembly overlap sequence can bind to the distal end of the insert polynucleotide via sequence in the first assembly overlap sequence that is complementary to the distal end of the insert polynucleotide. The second assembly overlap sequence can bind to the proximal end of an insert polynucleotide via sequence in the second assembly overlap sequence that is complementary to the reverse complement of the proximal end of an insert polynucleotide. In other words, the second assembly overlap sequence can comprise sequence that is identical to or the same as the proximal end of an insert polynucleotide or a portion thereof. The insert polynucleotides can be double stranded and the terms proximal or 5′ end and distal or 3′ end can be in reference to the top or sense strand.

The first assembly overlap sequence and the second assembly overlap sequence on a targeting polynucleotide or forward primer as provided herein can vary in length. The minimum length of the first and/or second assembly overlap sequence may be defined by a preferable Tm that is determined empirically. The first assembly overlap and/or the second assembly overlap sequence can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more nucleotides. The first assembly overlap and/or the second assembly overlap sequence can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 nucleotides in length. The first assembly overlap and/or the second assembly overlap sequence can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 nucleotides in length. The first and/or second assembly overlap sequences length can be in the range of 15 nucleotides-100 nucleotides for example up to 20, up to 25, up to 30, up to 35, up to 40, up to 45, up to 50, up to 55, up to 60, up to 65, up to 70, up to 75, up to 80 nucleotides, up to 85 nucleotides, up to 90 nucleotides, up to 95 nucleotides or up to 100 nucleotides. In one embodiment, the first assembly overlap sequence and the second assembly overlap sequence on a targeting polynucleotide or forward primer provided herein comprises 1 or more nucleotides that bind to the distal or 3′ end of an insert polynucleotide and the proximal or 5′ end of the insert polynucleotide, respectively. In another embodiment, the first assembly overlap sequence and the second assembly overlap sequence on a targeting polynucleotide or forward primer as provided herein comprises about 25 nucleotides that bind to the distal or 3′ end of an insert polynucleotide and the proximal or 5′ end of the insert polynucleotide, respectively. In some cases, the length of the first and second overlap sequence can be governed by the assembly method utilized. For example, when the assembly method is Golden Gate Assembly® as described herein, the first and second assembly overlap sequences can be the length of the overhangs generated by digestion of the Type IIS restriction sites with the appropriate Type IIS restriction enzyme. If the Type IIS restriction site is specific for a Type IIS restriction enzyme that generates four (4)-base overhangs, then the first and second assembly-overlap sequences can be four (4) bases long. The insert polynucleotides can be double-stranded, and the terms proximal end and distal end can be in reference to the top or sense strand.

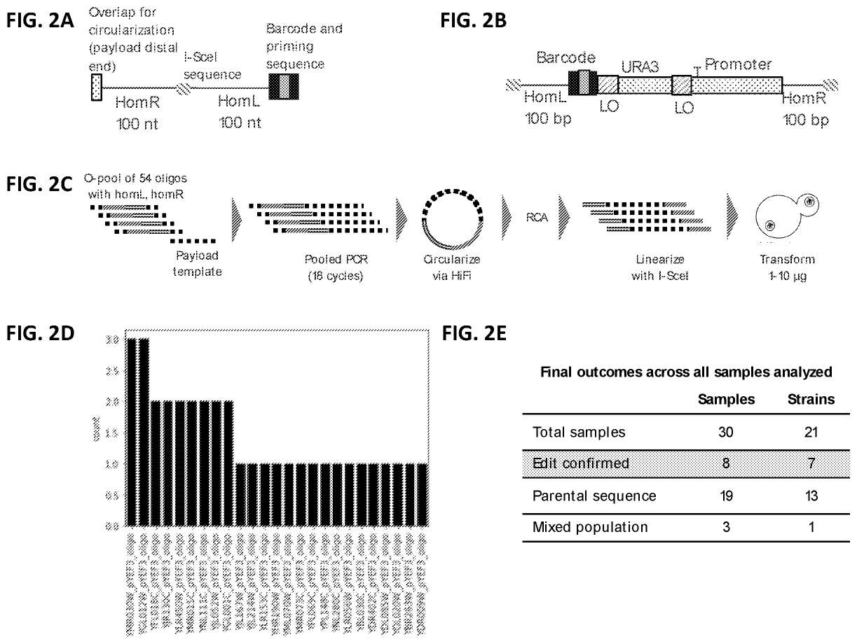

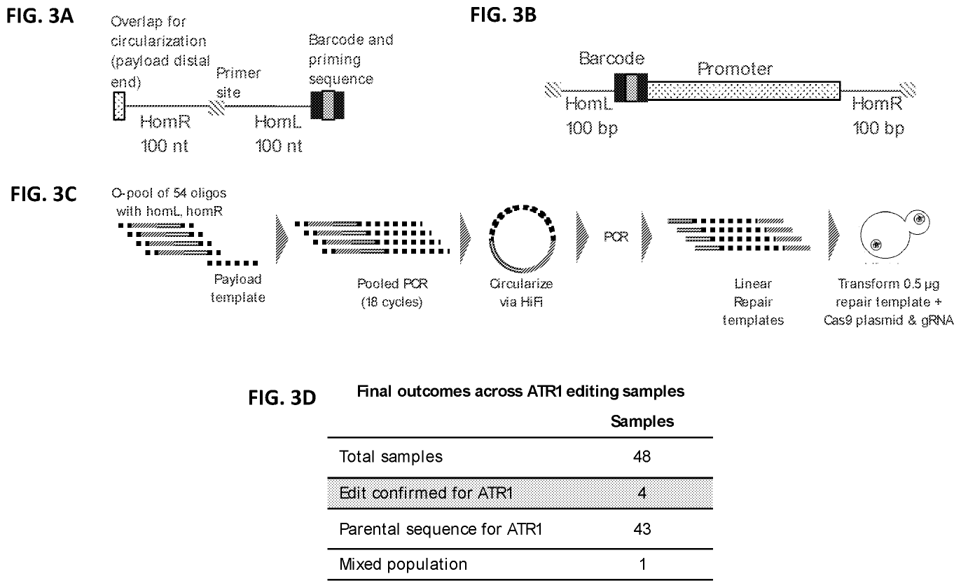

In some cases, as provided herein, the insert polynucleotide can comprise a barcode sequence such that one of the assembly overlap sequences can comprise sequence that binds (e.g., via complementarity) to said barcode sequence as shown, for example, in FIGS. 2A and 3A. In one embodiment, a proximal portion of an insert polynucleotide comprises a barcode sequence and a second assembly overlap sequence on a targeting polynucleotide comprises sequence that binds to all or a portion of said barcode sequence or a reverse complement thereof. The barcode sequence can comprise a sequence unique to each combination of payload sequence and first and second homology arms flanked by sequence universal to the barcode sequence present in each other payload sequence. The sequence universal to the barcode sequence present in each other payload sequence can then be used for amplifying or sequencing the unique sequence in each barcode.

In one embodiment, the linearization sequence present in a targeting polynucleotide as provided herein comprises one or more recognition sequences for one or more site-specific nucleases. In one embodiment, each targeting polynucleotide in a pool of targeting polynucleotides comprise one or more recognition sequences for one or more site-specific nucleases. In one embodiment, the linearization sequence present in a subset of targeting polynucleotides in a pool of targeting polynucleotides comprises one or more recognition sequences for one or more site-specific nucleases. In another embodiment, the linearization sequence present in a targeting polynucleotide as provided herein comprises one or more primer binding sites such that linearization can be facilitated by PCR. In another embodiment, the linearization sequence present in each targeting polynucleotide in a pool of targeting polynucleotides comprises one or more primer binding sites such that linearization can be facilitated by PCR. In one embodiment, the linearization sequence present in a subset of targeting polynucleotides in a pool of targeting polynucleotides comprises one or more primer binding sites such that linearization can be facilitated by PCR. In one embodiment, the linearization sequence present in a subset of targeting polynucleotides in a pool of targeting polynucleotides comprises one or more primer binding sites such that linearization can be facilitated by PCR, while the remainder of the targeting polynucleotides in a pool of targeting polynucleotides comprises one or more recognition sequences for one or more site-specific nucleases. In another embodiment, the linearization sequence present in a subset of targeting polynucleotides in a pool of targeting polynucleotides comprises one or more recognition sequences for one or more site-specific nucleases, while the remainder of the targeting polynucleotides in a pool of targeting polynucleotides comprises one or more primer binding sites such that linearization can be facilitated by PCR. The one or more primer binding sites can be common to each targeting polynucleotide comprising a linearization sequence that comprises one or more primer binding sites in a pool of targeting polynucleotides. At least one of the one or more primer binding sites in a targeting polynucleotide comprising a linearization sequence that comprises one or more primer binding sites is common to at least one of the one or more primer binding sites in each other targeting polynucleotide comprising a linearization sequence that comprises one or more primer binding sites in a pool of targeting polynucleotides. The primer pair directed to one of the one or more primer binding sites located between the first homology arm and the second homology arm is directed to the primer binding site common to each targeting polynucleotide comprising a linearization sequence that comprises one or more primer binding sites in the pool of targeting polynucleotides. At least one of the one or more primer binding sites in a targeting polynucleotide comprising a linearization sequence that comprises one or more primer binding sites is not found in any of the one or more primer binding sites in each other targeting polynucleotide comprising a linearization sequence that comprises one or more primer binding sites in a pool of targeting polynucleotides. The primer pair directed to one of the one or more primer binding sites located between the first homology arm and the second homology arm is directed to the primer binding site not found in any of the one or more primer binding sites in each other targeting polynucleotide comprising a linearization sequence that comprises one or more primer binding sites in the pool of targeting polynucleotides. At least one of the one or more primer binding sites in a targeting polynucleotide comprising a linearization sequence that comprises one or more primer binding sites is common to at least one of the one or more primer binding sites in a subset of other targeting polynucleotide comprising a linearization sequence that comprises one or more primer binding sites in a pool of targeting polynucleotides. The primer pair directed to one of the one or more primer binding sites located between the first homology arm and the second homology arm is directed to the primer binding site common to the subset of each targeting polynucleotide comprising a linearization sequence that comprises one or more primer binding sites in the pool of targeting polynucleotides. The one or more site-specific nuclease(s) recognition sequence can be one or more of a Type I restriction endonuclease(s), Type IIS restriction endonuclease(s), meganuclease(s), RNA-guided nuclease(s), DNA-guided nuclease(s), zinc-finger nuclease(s), TALEN(s) or nicking enzyme(s).

In embodiments where PCR is used to assemble a targeting polynucleotide with an insert polynucleotide as provided herein, each targeting polynucleotide serves as a forward primer. Further to this embodiment, a pool of targeting polynucleotides can be a pool of forward primers. Further to these embodiments, for each insert polynucleotide, a composition provided herein or a method provided herein comprises a forward primer or a plurality of forward primers and a reverse primer, wherein the forward primer or plurality of forward primers comprises from 5′ to 3′, a first assembly overlap sequence comprising sequence that binds to a distal end of the insert polynucleotide, the first homology arm, the linearization sequence, the second homology arm and a second assembly overlap sequence comprising sequence that binds to a proximal end of the insert polynucleotide, and wherein the reverse primer comprises sequence complementary to the distal end of the insert polynucleotide. The first assembly overlap sequence of any targeting polynucleotide and/or reverse primer can bind to the distal end of the insert polynucleotide via sequence in the first assembly overlap sequence and/or reverse primer that is complementary to the distal end of the insert polynucleotide. The second assembly overlap sequence of any targeting polynucleotide can bind to the proximal end of the insert polynucleotide via sequence in the second assembly overlap sequence that is complementary to the reverse complement of the proximal end of the insert polynucleotide. In other words, the second assembly overlap sequence can comprise sequence that is identical to or the same as the proximal end of the insert polynucleotide or a portion thereof. The forward primer can be from a pool of forward primers and/or the reverse primer can be from a pool of reverse primers. In one embodiment, each forward primer in the pool of forward primers is paired with a reverse primer from the pool of reverse primers such that each pair of forward and reverse primers comprise sequence complementary to at least one insert polynucleotide from a pool of insert polynucleotides. In one embodiment, each forward primer in a pool of forward primers comprises sequence to an identical or the same insert polynucleotide, while each reverse primer in a pool of reverse primers comprises sequence complementary to said identical or the same insert polynucleotide, but the first and second homology arms in each forward primer in the pool of forward primers comprises sequence directed to a different locus in the genome of a host cell than each other forward primer in the pool of forward primers. Further to this embodiment, the reverse primer can therefore be referred to as a common primer and be used with each forward primer from the pool of forward primers. The insert polynucleotides can be double-stranded, and the terms proximal end and distal end can be in reference to the top or sense strand.

### Insert Polynucleotides/Payload Sequences

In one embodiment, an insert polynucleotide for use in a composition, kit or method provided herein comprises one or more payload sequences. The one or more payload sequences can be located between the portions of the insert polynucleotides to which the first and second assembly overlap sequences from a targeting polynucleotide(s) as provided herein comprises sequence that binds thereto. The distal or 3′ end of an insert polynucleotide to which the first assembly overlap sequence from a targeting polynucleotide or forward primer can bind thereto (via complementarity) can be found within one of the one or more payload sequences. The distal or 3′ end of an insert polynucleotide to which the first assembly overlap sequence from a targeting polynucleotide or forward primer can bind thereto (via complementarity) can be found downstream of the one or more payload sequences. The proximal or 5′ end of an insert polynucleotide to which the second assembly overlap sequence from a targeting polynucleotide or forward primer can bind thereto (via complementarity) can be found within one of the one or more payload sequences. The proximal or 5′ end of an insert polynucleotide to which the second assembly overlap sequence from a targeting polynucleotide or forward primer can bind thereto (via complementarity) can be found upstream one of the one or more payload sequences. In embodiments utilizing reverse primers, the distal end of an insert polynucleotide to which the reverse primer comprises sequence that can bind thereto (via complementarity) can be found within one of the one or more payload sequences. In other embodiments utilizing reverse primers, the distal end of an insert polynucleotide to which the reverse primer comprises sequence that can bind thereto (via complementarity) can be found downstream one of the one or more payload sequences.

In one embodiment, each insert polynucleotide utilized in a composition and/or method provided herein is present on a plasmid. Further to this embodiment, the insert polynucleotide can be isolated or removed from the plasmid prior to be utilized in any of the editing or assembly methods provided herein. Isolation or removal of the insert polynucleotide can be accomplished by performing PCR using primers directed to the insert polynucleotide. In another embodiment, each insert polynucleotide utilized in a composition and/or method provided herein is a linear fragment of nucleic acid. The linear polynucleotide can be a gBlock 0. The insert polynucleotide can be double-stranded, and the terms proximal end and distal end can be in reference to the top or sense strand.

A payload sequence can be a random sequence. A payload sequence can be a marker sequence. The marker sequence can be any marker sequence known in the art. A payload sequence can be a gene or a portion thereof. The gene or portion thereof can be part of a metabolic or biochemical pathway. The gene or portion thereof can encode a protein or a domain thereof. A payload sequence can be whole or portions of promoters, genes, regulatory sequences, nucleic acid sequence encoding degrons, nucleic acid sequence encoding solubility tags, nucleic acid sequence encoding degradation tags, terminators, barcodes, regulatory sequences or portions thereof. In some cases, one or each of the one or more payload sequence can comprise a barcode sequence. The barcode sequence can comprise a sequence unique to each combination of payload sequence and first and second homology arms flanked by sequence universal to the barcode sequence present in each other payload sequence. The sequence universal to the barcode sequence present in each other payload sequence can be used for amplifying or sequencing the unique sequence in each barcode. In some cases, the insert polynucleotides can comprise one or more payload sequences and a selectable marker gene. The sequence for the selectable marker sequence can be flanked by direct repeat sequences that can facilitate looping out of the selectable marker sequence. The selectable marker sequence can be any selectable marker sequence known in the art. The selectable marker sequence can be selected from the group consisting of an antibiotic resistance gene, an auxotrophic marker, a colorimetric marker, a gene for a reporter protein and a directional marker. The reporter protein can be any protein whose presence in the host cell can be readily observed. The reporter protein can be any reporter protein known in the art such as, for example, a fluorescent protein (e.g., green fluorescent protein (GFP), mCherry, etc.), a chromoprotein or a luciferase.

In one embodiment, a payload sequence present within an insert polynucleotide can result in an insertion relative to the original locus targeted by the homology arms present in a targeting polynucleotide, a deletion of sequence relative to the original locus targeted by the homology arms present in a targeting polynucleotide, or a replacement of one sequence with another. In the case of an insertion or modification, the ‘payload’ can be the intended final sequence. In the case of a deletion, the ‘payload’ can be a marker sequence, a random sequence or no sequence.

In one embodiment, the insert polynucleotides are used in a pooled fashion. Further to this embodiment, each insert polynucleotide in a pool of insert polynucleotides can comprise a first assembly overlap sequence that comprises sequence that binds to (via complementarity) sequence (e.g., an assembly overlap sequence) at a distal end of a targeting polynucleotide and a second assembly overlap sequence that comprises sequence that binds to (via complementarity) sequence (e.g., an assembly overlap sequence) at a proximal end of a targeting polynucleotide.

The pool of insert polynucleotides can contain any number of unique insert polynucleotide sequences. The number of insert polynucleotides can be at least, at most, or about 1, 5, 10, 25, 50, 75, 100, 125, 150, 175, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10,000, 20, 000, 30,000, 40,000, 50,000, 75,000, 100,000, 150,000, 200,000 or 250,000 unique insert polynucleotides with or without a payload sequence.

A payload sequence can be at most or at least 1, 2, 3, 4, 5, 6, 7, 8, 9, 10, 11 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 150, 200, 250, 300, 350, 400, 450, 500, 600, 700, 800, 900, 1000, 2000, 3000, 4000, 5000, 6000, 7000, 8000, 9000 or 10,000 nucleotides in length. In some cases, the payload sequence can be 0 nucleotides in length. A payload sequence can be at a length such that when incorporated into an insert polynucleotide, the entire insert polynucleotide can be chemically synthesized. The synthesis can be an array-based or column-based synthesis method as known in the art. The insert polynucleotide can be a gBlock®. An insert polynucleotide that can be synthesized can be up to about 1, 5, 10, 20, 30, 40, 50, 60, 70, 80, 90, 100, 110, 120, 130, 140, 150, 160, 170,180, 190, 200, 210, 220, 250, 300, 350, 400 or more nucleotides in length.

In one embodiment, each insert polynucleotide comprises one or more payload sequences such that each insert polynucleotide in a pool of insert polynucleotides comprises a different one or more payload sequences from the one or more payload sequences in each other insert polynucleotide in said pool.

In another embodiment, each insert polynucleotide comprises one or more payload sequences such that each insert polynucleotide in a pool of insert polynucleotides comprises the same one or more payload sequences as the one or more payload sequences in each other insert polynucleotide in said pool.

In yet another embodiment, a subset of insert polynucleotides in a pool of insert polynucleotides comprises one or more payload sequences that are the same one or more payload sequences as the one or more payload sequences in each other insert polynucleotide in said subset.

In still another embodiment, a subset of insert polynucleotides in a pool of insert polynucleotides comprises one or more payload sequences that are different one or more payload sequences as the one or more payload sequences in each other insert polynucleotide in the remainder of the pool.

### Cloning Methods

As described herein, a composition comprising targeting polynucleotides as well as insert polynucleotides and, optionally, reverse primers can be assembled into a library of nucleic acids comprising first and second homology arms with an insert polynucleotide therebetween. Assembly of the targeting polynucleotides with the insert polynucleotides as provided herein can be performed by any enzymatic or chemical method of joining two DNA molecules known in the art. The assembly method can be either an in vitro or in vivo cloning method. For the assembly of large DNA molecules, the final steps of the assembly may be conducted in vivo, such as in a yeast host cell. The balance between use of in vitro and in vivo assembly steps can be determined by the practicality of the method with regard to the nature of the nucleic acid molecules to be assembled. The assembly method can be selected from selected from the group consisting of splicing and overlap-extension PCR (SOE-PCR), Uracil-specific excision reagent (USER) cloning, restriction-ligation, blunt-end ligation, overlap based assembly method and recombination-based method.

In one embodiment, assembly of the targeting polynucleotides with the insert polynucleotides and, optionally, reverses primers, is performed using an in vitro cloning method. The in vitro cloning method can be any in vitro cloning method that employs overlap assembly known in the art. The in vitro cloning method used in the methods provided herein can be selected from infusion cloning (Clontech®), Golden Gate Assembly®, Gateway Assembly®, Gibson Assembly®, and NEB HIFI Assembly® or any other suitable in vitro cloning method known in the art. Infusion cloning can entail mixing a first pool of targeting polynucleotides as provided herein and a second pool of insert polynucleotides as described herein with the infusion cloning reagent and then transforming the resultant assemblies into an E. coli cloning host cell. The in vitro cloning method can be any of the overlap assembly methods described in U.S. Pat. No. 8,968,999, which is herein incorporated by reference in its entirety. The in vitro cloning method can be any of the overlap assembly methods described in US20160060671, which is herein incorporated by reference in its entirety. The in vitro cloning method can be the Gibson Assembly method described in Jun Urano, Ph.D. and Christine Chen, Ph.D., Gibson Assembly® Primer-Bridge End Joining (PBnJ) Cloning, Synthetic Genomics Application Note, which is herein incorporated by reference in its entirety. In one embodiment, a composition comprising pools of targeting polynucleotides and insert polynucleotides are joined using a 5′-3′ exonuclease; and a strand-displacing polymerase also present in the composition. The composition can also comprise a buffer containing a potassium salt such as potassium chloride in a concentration range of 7 mM-150 mM, for example, 20 mM-50 mM. A sodium salt (e.g., sodium chloride) in the range of 10 mM-100 mM such as 20 mM may also be used in addition to potassium salt. In some embodiments, the composition does not contain a crowding agent such as polyethylene glycol (PEG), Ficoll, or dextran. In some embodiments, the composition comprises a single stranded (ss) binding protein. A ss DNA binding protein for use in the composition may be E. coli recA, T7 gene 2.5 product, RedB (from phage lambda) or RecT (from Rac prophage), ET SSB (extreme thermostable single-stranded DNA binding protein) or any other ss DNA binding proteins known in the art could be used in the composition. The inclusion of a ss binding protein can improve the efficiency of assembly particularly for nucleic acid fragments with longer overlap sequences (e.g. at least 20 nucleotides) than would be otherwise occur in the absence of ss binding protein as measured by colony number. In some embodiments, the composition does not contain a non-strand displacing polymerase.

In another embodiment, a composition comprising targeting polynucleotides and insert polynucleotides and, optionally, reverse primers are joined using an isolated non-thermostable 5′ to 3′ exonuclease that lacks 3′ exonuclease activity, a crowding agent, a non-strand-displacing DNA polymerase with 3′ exonuclease activity, or a mixture of said DNA polymerase with a second DNA polymerase that lacks 3′ exonuclease activity, and a ligase. The composition can further comprise a mixture of dNTPs, and a suitable buffer, under conditions that are effective for joining the polynucleotides. In some embodiment, the composition can further comprise a crowding agent. The crowding agent can be selected from polyethylene glycol (PEG), dextran or Ficoll. In one embodiment, the crowding agent is PEG. The PEG can be used at a concentration of from about 3 to about 7% (weight/volume). The PEG can be selected from PEG-200, PEG-4000, PEG-6000, PEG-8000 or PEG-20,000. In some embodiments, the exonuclease of is a T5 exonuclease and the contacting is under isothermal conditions, and/or the crowding agent is PEG, and/or the non-strand-displacing DNA polymerase is PHUSION® DNA polymerase or VENTR® DNA polymerase, and/or a Taq ligase.

In one embodiment, assembly of the targeting polynucleotides with the insert polynucleotides, and, optionally, reverse primers is performed using an in vivo cloning method. The in vivo cloning method can be any in vivo cloning method known in the art. The in vivo cloning method can be a homologous recombination mediated cloning method. The in vivo cloning method used in the methods provided herein can be selected from E. coli (RecA-dependent, RecA-independent or Red/ET-dependent) homologous recombination, Overlap Extension PCR and Recombination (OEPR) cloning, yeast homologous recombination, and Transformation-associated recombination (TAR) cloning and gene assembly in Bacillus as described in Tsuge, Kenji et al. “One step assembly of multiple DNA fragments with a designed order and orientation in Bacillus subtilis plasmid.” Nucleic acids research vol. 31,21 (2003): e133, which is herein incorporated by reference.

### Applications

The composition and assembly methods provided herein can be used to construct any desired assembly, such as plasmids, genes, metabolic pathways, minimal genomes, partial genomes, genomes, chromosomes, extrachromosomal nucleic acids, for example, cytoplasmic organelles, such as mitochondria (animals), and in chloroplasts and plastids (plants), and the like.

The compositions and assembly methods provided herein can be used to generate libraries of nucleic acid molecules, and methods to use modified whole or partial nucleic acid molecules as generated therefrom. The libraries can contain 2 or more variants, and said multiple variants, can be screened for members having desired characteristics, such as high production levels of desired products of interest, enhanced functionality of the product of interest, or decreased functionality (if that is advantageous). Such screening may be done by high throughput methods, which may be robotic/automated as provided herein.

The disclosure also further includes products made by the compositions and assembly methods provided herein, for example, the resulting assembled synthetic genes or genomes (synthetic or naturally occurring) and modified optimized genes and genomes, and the use(s) thereof.

The compositions and assembly methods provided herein can have a wide variety of applications, permitting, for example, the design of pathways for the synthesis of desired products of interest or optimization of one or more sequences whose gene products play a role in the synthesis or expression of a desired product. The compositions and assembly methods provided herein can also be used to generate optimized sequences of a gene or expression thereof or to combine one or more functional domains or motifs of protein encoded by a gene. The gene can be part of a biochemical or metabolic pathway. The biochemical or metabolic pathway can produce a desired product of interest.

The desired product of interest can be any molecule that can be assembled in a cell culture, eukaryotic or prokaryotic expression system or in a transgenic animal or plant. Thus, the nucleic acid molecules or libraries thereof that result from the deterministic assembly methods provided herein may be employed in a wide variety of contexts to produce desired products of interest. In some cases, the product of interest may be a small molecule, enzyme, peptide, amino acid, organic acid, synthetic compound, fuel, alcohol, etc. For example, the product of interest or biomolecule may be any primary or secondary extracellular metabolite. The primary metabolite may be, inter alia, ethanol, citric acid, lactic acid, glutamic acid, glutamate, lysine, threonine, tryptophan and other amino acids, vitamins, polysaccharides, etc. The secondary metabolite may be, inter alia, an antibiotic compound like penicillin, or an immunosuppressant like cyclosporin A, a plant hormone like gibberellin, a statin drug like lovastatin, a fungicide like griseofulvin, etc. The product of interest or biomolecule may also be any intracellular component produced by a host cell, such as: a microbial enzyme, including catalase, amylase, protease, pectinase, glucose isomerase, cellulase, hemicellulase, lipase, lactase, streptokinase, and many others. The intracellular component may also include recombinant proteins, such as: insulin, hepatitis B vaccine, interferon, granulocyte colony-stimulating factor, streptokinase and others. The product of interest may also refer to a protein of interest.

### Pathway Assembly

In one embodiment, the compositions and methods provided herein are used to assemble a gene or a variant thereof. The gene or variant thereof can encode a protein that is part of a metabolic or biochemical pathway. The variant can be a codon optimized version or mutated version of said gene. The metabolic or biochemical pathway can produce a product of interest as provided herein. In one embodiment, the gene sequence or variant thereof can be present as a payload sequence within an insert polynucleotide as provided herein. The pair of homology arms in each targeting polynucleotide can comprise sequence such that when assembled with said insert polynucleotide can serve to facilitate targeting of and insertion into a locus in a genetic element (e.g., genome, plasmid, etc.) within a host cell using a gene editing method as provided herein. The locus can be a specific locus or a random locus. Alternatively, the pair of homology arms in each targeting polynucleotide can comprise sequence that when assembled with said insert polynucleotide can serve to facilitate further assembly of the resultant assembly with other assemblies generated using the methods provided herein. The other assemblies can comprise one or more additional genes present within the same metabolic or biochemical pathway and is this way facilitate the assembly of said metabolic or biochemical pathway. All of the genes or variants thereof can be assembled using the technique described herein of overlapping sequences on a single vector for a particular metabolic or biochemical pathway, or independent vectors for each member of said pathway can be employed by mixing the vectors for each member in successive transformation mixtures. The assembly of a targeting polynucleotide with an insert polynucleotide and, optionally, a reverse primer can be accomplished via assembly overlap sequences present in each of the targeting polynucleotides using the assembly overlap methods provided herein. In some cases, the targeting polynucleotide can further comprise sequence of a regulatory or control element or a portion thereof that can govern an aspect of the gene or variant thereof or the protein encoded thereby such as the transcription, translation, solubility, or degradation thereof. The regulatory or control element can be a promoter, terminator, solubility tag, degradation tag or degron.

In another embodiment, the gene sequence or variant thereof is spread across a pair of homology arms in each targeting polynucleotide and an insert polynucleotide located there between. By suitable assembly overlap segments on each of the polynucleotides, a mixture containing all of the polynucleotides can be assembled in the correct order in a single reaction mixture using overlap assembly as provided herein. The resultant will be full-length coding sequences of the gene or variant thereof. The pairs of first and second polynucleotides can further comprise sequence such that when assembled with said insert polynucleotide can serve to facilitate targeting of and insertion into a locus in a genetic element (e.g., genome, plasmid, etc.) within a host cell using a gene editing method as provided herein. The locus can be a specific locus or a random locus. Alternatively, the targeting polynucleotide can further comprise sequence that when assembled with said insert polynucleotide can serve to facilitate further assembly of the resultant assembly with other assemblies generated using the methods provided herein. The other assemblies can comprise one or more additional genes present within the same metabolic or biochemical pathway and is this way facilitate the assembly of said metabolic or biochemical pathway. All of the genes or variants thereof can be assembled using the technique described herein of overlapping sequences for a particular metabolic or biochemical pathway, or independent genes or variants for each member of said pathway can be employed by mixing the linear insert polynucleotides for each member in successive transformation mixtures. In some cases, the targeting polynucleotide can further comprise sequence of a regulatory or control element that can govern an aspect of the gene or variant thereof or the protein encoded thereby such as the transcription, translation, solubility, or degradation thereof. The regulatory or control element can be a promoter, terminator, solubility tag, degradation tag or degron.

In still another embodiment, the compositions and methods provided herein are used to assemble or combine nucleic acid sequence that encode motifs or domains of a target protein. The nucleic acid sequence encoding a particular motif or domain of a target protein can be spread across a targeting polynucleotide and an insert polynucleotide located therebetween. The nucleic acid sequence encoding a particular motif or domain of a target protein can be present on a targeting polynucleotide while a second motif or domain of the target protein can be present on an insert polynucleotide and an assembly overlap method provided herein can be used to join said first and second motif or domain of the target protein.

### Gene Editing

As described herein, a composition comprising a pool of targeting polynucleotides as well as a pool of insert polynucleotides and, optionally, a pool of reverse primers can be assembled into a library of nucleic acids comprising first and second homology arms with an insert polynucleotide therebetween that can be subsequently utilized to modify the genetic content of a host cell. As provided herein, the library of nucleic acids can comprise payloads that can be control elements (e.g., promoters, terminators, solubility tags, degradation tags or degrons), modified forms of genes (e.g., genes with desired SNP(s)), antisense nucleic acids, and/or one or more genes that are part of a metabolic or biochemical pathway. In one embodiment, the modification entails gene editing of the host cell. The gene editing can entail editing the genome of the host cell and/or a separate genetic element present in the host cell such as, for example, a plasmid or cosmid. The gene editing method that can utilize nucleic acid assemblies generated using the methods and compositions as provided herein can be any gene editing method or system known in the art and can be selected based on the host for which gene editing is desired. Non-limiting examples of gene editing methods include homologous recombination, CRISPR based gene editing, Transcription activator-like effector nucleases (TALENS) based gene editing, FOK1 based gene editing methods, or other gene editing methods that utilize endonucleases known in the art.

### Homologous Recombination

In one embodiment, the gene editing method used in conjunction with the nucleic acid assemblies generated using the compositions and methods provided herein is a homologous recombination-based method known in the art. The homologous recombination-based method can be selected from single-crossover homologous recombination, double-crossover homologous recombination, or lambda red recombineering. Further to this embodiment, the first and second homology arms in a targeting polynucleotide each comprise sequence directed to or complementary to a desired locus in a nucleic acid element (e.g., genome, plasmid or cosmid) of a host cell and thereby direct an insert polynucleotide located therebetween to a desired locus in the genetic element (e.g., genome, cosmid or plasmid) of the host cell. Accordingly, the sequence directed to or complementary to a desired locus present in the targeting polynucleotide can be used to determine the location(s) in the genome, cosmid or plasmid that will be targeted for editing. As exemplified in FIG. 1A, each targeting polynucleotide comprises from 5′ to 3′, a first assembly overlap sequence comprising sequence that binds to (e.g., via complementarity) a distal end of an insert polynucleotide, a first homology arm, a linearization sequence, a second homology arm and a second assembly overlap sequence comprising sequence that binds to (e.g., via complementarity) a proximal end or reverse complement thereof of the insert polynucleotide (primer bind-payload sequence in FIG. 1B). In embodiments where the method employs or the composition comprises a reverse primer or pool of reverse primers (e.g., FIG. 1C), each targeting polynucleotide serves as a forward primer.

In one embodiment, for each pair of homology arms in a targeting polynucleotide in a pool of targeting polynucleotides, the sequence that is complementary to a desired locus in the pair is complementary to a different target locus in a host cell as compared to the homology arms in each other targeting polynucleotide in the pool.

In another embodiment, for each pair of homology arms in a targeting polynucleotide in a pool of targeting polynucleotides, the sequence that is complementary to a desired locus in the pair is complementary to an identical or a same target locus in a host cell as compared to the homology arms in each other targeting polynucleotide in the pool.

In another embodiment, for each pair of homology arms in a targeting polynucleotide in a pool of targeting polynucleotides, the sequence that is complementary to a desired locus in the pair is complementary to an identical or a same target locus in a host cell as compared to the homology arms in a subset of other targeting polynucleotides in the pool.

In another embodiment, for each pair of homology arms in a targeting polynucleotide in a pool of targeting polynucleotides, the sequence that is complementary to a desired locus in the pair is complementary to a different target locus in a host cell as compared to the homology arms in a subset of other targeting polynucleotides in the pool.

Loop-In/Loop-Out

In some embodiments, the present disclosure teaches methods of looping out selected regions of DNA from the host organisms. The looping out method can be as described in Nakashima et al. 2014 “Bacterial Cellular Engineering by Genome Editing and Gene Silencing.” Int. J. Mol. Sci. 15(2), 2773-2793. Looping out deletion techniques are known in the art and are described in Tear et al. 2014 “Excision of Unstable Artificial Gene-Specific inverted Repeats Mediates Scar-Free Gene Deletions in Escherichia coli.” Appl. Biochem. Biotech. 175:1858-1867. The looping out methods used in the methods provided herein can be performed using single-crossover homologous recombination or double-crossover homologous recombination. In one embodiment, looping out of selected regions can entail using single-crossover homologous recombination.

In one embodiment, a composition provided herein comprises a pool of targeting polynucleotides comprising left/right homology arms, a pool of insert polynucleotides and, optionally, a pool of reverse primers such that assembly of the pools of targeting polynucleotides and insert polynucleotides alone or in combination with the optional pool of reverse primers using an assembly method as provided herein generates circularized nucleic acid assemblies that can act as loop out vectors. In one embodiment, single-crossover homologous recombination is used between a loop-out vector and the host cell genome in order to loop-in said vector. The vector could comprise a marker that facilitates selection of looped-out clones after the loop-in step. In another embodiment, double-crossover homologous recombination is used between a loop-out vector and the host cell genome in order to integrate said vector. The insert sequence within the loop-out vector can be designed with a sequence, which is a direct repeat of an existing or introduced nearby host sequence, such that the direct repeats flank the region of DNA slated for looping and deletion. The insert sequence could further comprise a marker that facilitates selection of looped-out clones. Once inserted, cells containing the loop out vector can be counter selected for deletion of the selection region.

In one aspect provided herein, polynucleotides or polynucleotide libraries generated using the compositions and/or methods provided herein can be used in a gene editing method that can entail the use of sets of proteins from one or more recombination systems. Said recombination systems can be endogenous to the microbial host cell or can be introduced heterologously. The sets of proteins of the one or more heterologous recombination systems can be introduced as nucleic acids (e.g., as plasmid, linear DNA or RNA, or integron) and be integrated into the genome of the host cell or be stably expressed from an extrachromosomal element. The sets of proteins of the one or more heterologous recombination systems can be introduced as RNA and be translated by the host cell. The sets of proteins of the one or more heterologous recombination systems can be introduced as proteins into the host cell. The sets of proteins of the one or more recombination systems can be from a lambda red recombination system, a RecET recombination system, a Red/ET recombination system, any homologs, orthologs or paralogs of proteins from a lambda red recombination system, a RecET recombination system, or Red/ET recombination system or any combination thereof. The recombination methods and/or sets of proteins from the RecET recombination system can be any of those as described in Zhang Y., Buchholz F., Muyrers J. P. P. and Stewart A. F. “A new logic for DNA engineering using recombination in E. coli.” Nature Genetics 20 (1998) 123-128; Muyrers, J. P. P., Zhang, Y., Testa, G., Stewart, A. F. “Rapid modification of bacterial artificial chromosomes by ET-recombination.” Nucleic Acids Res. 27 (1999) 1555-1557; Zhang Y., Muyrers J. P. P., Testa G. and Stewart A. F. “DNA cloning by homologous recombination in E. coli.” Nature Biotechnology 18 (2000) 1314-1317 and Muyrers J P et al., “Techniques: Recombinogenic engineering—new options for cloning and manipulating DNA” Trends Biochem Sci. 2001 May;26(5):325-31, which are herein incorporated by reference. The sets of proteins from the Red/ET recombination system can be any of those as described in Rivero-Muller, Adolfo et al. “Assisted large fragment insertion by Red/ET-recombination (ALFIRE)—an alternative and enhanced method for large fragment recombineering” Nucleic acids research vol. 35,10 (2007): e78, which is herein incorporated by reference.

Lambda RED Mediated Gene Editing

As provided herein, gene editing as described herein can be performed using Lambda Red-mediated homologous recombination as described by Datsenko and Wanner, PNAS USA 97:6640-6645 (2000), the contents of which are hereby incorporated by reference in their entirety.

To use the lambda red recombineering system to modify target DNA, a linear donor DNA sequence (either dsDNA or ssDNA) can be introduced (e.g., via electroporation) into a host cell (e.g., E. coli) expressing the set of proteins from the lambda red recombination system. The linear donor DNA sequence can be an assembly comprising a pair of homology arms with an insert polynucleotide located therebetween generated using the methods and compositions provided herein. The set of proteins from the lambda red recombination system can comprise the exo, beta or gam proteins or any combination thereof. Gam can prevent both the endogenous RecBCD and SbcCD nucleases from digesting the linear donor DNA (either dsDNA or ssDNA) introduced into a microbial host cell, while exo is a 5′→3′ dsDNA-dependent exonuclease that can degrade linear dsDNA starting from the 5′ end and generate 2 possible products (i.e., a partially dsDNA duplex with single-stranded 3′ overhangs or a ssDNA whose entire complementary strand was degraded) and beta can protect the ssDNA created by Exo and promote its annealing to a complementary ssDNA target in the cell. Beta expression can be required for lambda red based recombination with an ssDNA oligo substrate as described at blog.addgene.org/lambda-red-a-homologous-recombination-based-technique-for-genetic-engineering, the contents of which are herein incorporated by reference.

As described herein, the linear donor DNA sequence or substrate (either dsDNA or ssDNA) can be an assembly comprising a pair of homology arms with an insert polynucleotide located therebetween generated using the methods and compositions provided herein. The pair of homology arms can comprise genomic targeting sequences that target said donor DNA substrate to a specific locus in the genome of the host cell. The enzymes of the lambda red system then catalyze the homologous recombination of the substrate with the target DNA sequence. The homology arms on the donor DNA substrate can direct to the donor DNA substrate to the target site for recombination with only about ˜50 nucleotides of homology to the target site. As described at b log . addgene. org/lamb da-red-a-homo log ous-recombinati on-bas ed-technique-for-g enetic-engineering, whether a linear dsDNA or ssDNA substrate is used can depend on the goal of the experiment. dsDNA substrate may be best for insertions or deletions greater than approximately 20 nucleotides, while ssDNA substrate may be best for point mutations or changes of only a few base pairs.

dsDNA substrate can be made using the compositions and methods provided herein such that the linear insert polynucleotides comprise about 50 base pairs of homologous sequence (i.e., homology arms) to the targeted insert site on opposing terminal ends. The dsDNA payloads present within the linear insert polynucleotides can include large insertions or deletions, including selectable DNA fragments, such as antibiotic resistance genes, as well as non-selectable DNA fragments, such as gene replacements and tags.

ssDNA substrates can be also be made using the compositions and methods provided herein such that the linear insert polynucleotides comprise about 50 base pairs of homologous sequence (i.e., homology arms) to the targeted insert site on opposing terminal ends and can have the desired payload sequence(s) (i.e., within the linear insert polynucleotide).

ssDNA substrate can be more efficient than dsDNA with a recombination frequency between 0.1% to 1% and can be increased to as high as 25-50% by designing substrates that avoid activating the methyl -directed mismatch repair (MMR) system. MMR'sjob is to correct DNA mismatches that occur during DNA replication. Activation of MMR can be avoided by: 1) using a strain of bacteria that has key MMR proteins knocked out or 2) specially design ssDNA substrates to avoid MMR: 1) E. coil with inactivated MMR: Using E. coli with inactive MMR is definitely the easier of the two options, but these cells are prone to mutations and can have more unintended changes to their genomes. 2) Designing ssDNA substrates that avoid MMR activation: In one embodiment, a C/C mismatch at or within 6 base pairs of the edit site is introduced. In another embodiment, the desired change is flanked with 4-5 silent changes in the wobble codons, i.e. make changes to the third base pair of the adjacent 4-5 codons that alter the nucleotide sequence but not the amino acid sequence of the translated protein. These changes can be 5′ or 3′ of the desired change.

In one embodiment, the polynucleotides or polynucleotide libraries generated using the compositions and/or methods provided herein can be used in a gene editing method that is implemented in a microbial host cell that already stably expresses lambda red recombination genes such as the DY380 strain described at blog.addgene.org/lambda-red-a-homologous-recombination-based-technique-for-genetic-engineering, the contents of which are herein incorporated by reference. Other bacterial strains that comprise components of the lambda red recombination system and can be utilized with the nucleic acid assemblies generated using the compositions and methods provided herein can be found in Thomason et al (Recombineering: Genetic Engineering in Bacteria Using Homologous Recombination. Current Protocols in Molecular Biology. 106:V:1.16:1.16.1-1.16.39) and Sharan et al (Recombineering: A Homologous Recombination-Based Method of Genetic Engineering. Nature protocols. 2009;4(2):206-223), the contents of each of which are herein incorporated by reference.

As provided herein, the set of proteins of the lambda red recombination system can be introduced into the microbial host cell prior to implementation of any of the editing methods known in the art and/or provided herein. Genes for each of the proteins of the lambda red recombination system can be introduced on nucleic acids (e.g., as plasmids, linear DNA or RNA, a mini-λ, a lambda red prophage or integrons) and be integrated into the genome of the host cell or expressed from an extrachromosomal element. In some cases, each of the components (i.e., exo, beta, gam or combinations thereof) of the lambda red recombination system can be introduced as an RNA and be translated by the host cell. In some cases, each of the components (i.e., exo, beta, gam or combinations thereof) of the lambda red recombination system can be introduced as a protein into the host cell.

In one embodiment, genes for the set of proteins of the lambda red recombination system are introduced on a plasmid. The set of proteins of the lambda red recombination system on the plasmid can be under the control of a promoter such as, for example, the endogenous phage pL promoter. In one embodiment, the set of proteins of the lambda red recombination system on the plasmid is under the control of an inducible promoter. The inducible promoter can be inducible by the addition or depletion of a reagent or by a change in temperature. In one embodiment, the set of proteins of the lambda red recombination system on the plasmid is under the control of an inducible promoter such as the IPTG-inducible lac promoter or the arabinose-inducible pBAD promoter. A plasmid expressing genes for the set of proteins of the lambda red recombination system can also express repressors associated with a specific promoter such as, for example, the lad, araC or cI857 repressors associated with the IPTG-inducible lac promoter, the arabinose-inducible pBAD promoter and the endogenous phage pL promoters, respectively.

In one embodiment, genes for the set of proteins of the lambda red recombination system are introduced on a mini-λ, which a defective non-replicating, circular piece of phage DNA, that when introduced into microbial host cell, integrates into the genome as described at blog. addgene. org/lambda-red-a-homol ogous-recombinati on-based-technique-for-genetic-engineering, the contents of which are herein incorporated by reference.

In one embodiment, genes for the set of proteins of the lambda red recombination system are introduced on a lambda red prophage, which can allow for stable integration of the lambda red recombination system into a microbial host cell such as described at blog.addgene.org/lambda-red-a-homologous-recombination-based-technique-for-genetic-engineering, the contents of which are herein incorporated by reference.

CRISPR Mediated Gene Editing

In one aspect provided herein, a genetic element (e.g., genome, cosmid, or plasmid) of a host cell can be modified with a linear insert polynucleotide generated using any of the compositions or methods provided herein by CRISPR.

The CRISPR/Cas system is a prokaryotic immune system that confers resistance to foreign genetic elements such as those present within plasmids and phages and that provides a form of acquired immunity. CRISPR stands for Clustered Regularly Interspaced Short Palindromic Repeat, and Cas stands for CRISPR-associated system and refers to the small cas genes associated with the CRISPR complex.

CRISPR-Cas systems are most broadly characterized as either Class 1 or Class 2 systems. The main distinguishing feature between these two systems is the nature of the Cas-effector module. Class 1 systems require assembly of multiple Cas proteins in a complex (referred to as a “Cascade complex”) to mediate interference, while Class 2 systems use a large single Cas enzyme to mediate interference. Each of the Class 1 and Class 2 systems are further divided into multiple CRISPR-Cas types based on the presence of a specific Cas protein. For example, the Class 1 system is divided into the following three types: Type I systems, which contain the Cas3 protein; Type III systems, which contain the Cas10 protein; and the putative Type IV systems, which contain the Csf1 protein, a Cas8-like protein. Class 2 systems are generally less common than Class 1 systems and are further divided into the following three types: Type II systems, which contain the Cas9 protein; Type V systems, which contain Cas12a protein (previously known as Cpf1, and referred to as Cpf1 herein), Cas12b (previously known as C2c1), Cas12c (previously known as C2c3), Cas12d (previously known as CasY), and Cas12e (previously known as CasX); and Type VI systems, which contain Cas13a (previously known as C2c2), Cas13b, and Cas13c. Pyzocha et al., ACS Chemical Biology, Vol. 13 (2), pgs. 347-356. In one embodiment, the CRISPR-Cas system for use in the editing methods provided herein is a Class 2 system. In one embodiment, the CRISPR-Cas system for use in the editing methods provided herein is a Type II, Type V or Type VI Class 2 system. In one embodiment, the CRISPR-Cas system for use in the editing methods provided herein is selected from Cas9, Cas12a, Cas12b, Cas12c, Cas12d, Cas12e, Cas13a, Cas13b, Cas13c or homologs, orthologs or paralogs thereof.

CRISPR systems used in editing methods disclosed herein can comprise a Cas effector module comprising one or more nucleic acid guided CRISPR-associated (Cas) nucleases, referred to herein as Cas effector proteins. In some embodiments, the Cas proteins can comprise one or multiple nuclease domains. A Cas effector protein can target single stranded or double stranded nucleic acid molecules (e.g. DNA or RNA nucleic acids) and can generate double strand or single strand breaks. In some embodiments, the Cas effector proteins are wild-type or naturally occurring Cas proteins. In some embodiments, the Cas effector proteins are mutant Cas proteins, wherein one or more mutations, insertions, or deletions are made in a WT or naturally occurring Cas protein (e.g., a parental Cas protein) to produce a Cas protein with one or more altered characteristics compared to the parental Cas protein.

In some instances, the Cas protein is a wild-type (WT) nuclease. Non-limiting examples of suitable Cas proteins for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, MAD1-20, SmCsm1, homologues thereof, orthologues thereof, variants thereof, mutants thereof, or modified versions thereof. Suitable nucleic acid guided nucleases (e.g., Cas 9) can be from an organism from a genus, which includes but is not limited to: Thiomicrospira, Succinivibrio, Candidatus, Porphyromonas, Acidomonococcus, Prevotella, Smithella, Moraxella, Synergistes, Francisella, Leptospira, Catenibacterium, Kandleria, Clostridium, Dorea, Coprococcus, Enterococcus, Fructobacillus, Weissella, Pediococcus, Corynebacter, Sutterella, Legionella, Treponema, Roseburia, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Alicyclobacillus, Brevibacilus, Bacillus, Bacteroidetes, Brevibacilus, Carnobacterium, Clostridiaridium, Clostridium, Desulfonatronum, Desulfovibrio, Helcococcus, Leptotrichia, Listeria, Methanomethyophilus, Methylobacterium, Opitutaceae, Paludibacter, Rhodobacter, Sphaerochaeta, Tuberibacillus, and Campylobacter. Species of organism of such a genus can be as otherwise herein discussed.

Suitable nucleic acid guided nucleases (e.g., Cas9) can be from an organism from a phylum, which includes but is not limited to: Firmicute, Actinobacteria, Bacteroidetes, Proteobacteria, Spirochetes, and Tenericutes. Suitable nucleic acid guided nucleases can be from an organism from a class, which includes but is not limited to: Erysipelotrichia, Clostridia, Bacilli, Actinobacteria, Bacteroidetes, Flavobacteria, Alphaproteobacteria, Betaproteobacteria, Gammaproteobacteria, Deltaproteobacteria, Epsilonproteobacteria, Spirochaetes, and Mollicutes. Suitable nucleic acid guided nucleases can be from an organism from an order, which includes but is not limited to: Clostridiales, Lactobacillales, Actinomycetales, Bacteroidales, Flavobacteriales, Rhizobiales, Rhodospirillales, Burkholderiales, Neisseriales, Legionellales, Nautiliales, Campylobacterales, Spirochaetales, Mycoplasmatales, and Thiotrichales. Suitable nucleic acid guided nucleases can be from an organism from within a family, which includes but is not limited to: Lachnospiraceae, Enterococcaceae, Leuconostocaceae, Lactobacillaceae, Streptococcaceae, Peptostreptococcaceae, Staphylococcaceae, Eubacteriaceae, Corynebacterineae, Bacteroidaceae, Flavobacterium, Cryomoorphaceae, Rhodobiaceae, Rhodospirillaceae, Acetobacteraceae, Sutterellaceae, Neisseriaceae, Legionellaceae, Nautiliaceae, Campylobacteraceae, Spirochaetaceae, Mycoplasmataceae, and Francisellaceae.

Other nucleic acid guided nucleases (e.g., Cas9) suitable for use in the methods, systems, and compositions of the present disclosure include those derived from an organism such as, but not limited to: Thiomicrospira sp. XS5, Eubacterium rectale, Succinivibrio dextrinosolvens, Candidatus Methanoplasma termitum, Candidatus Methanomethylophilus alvus, Porphyromonas crevioricanis, Flavobacterium branchiophilum, Acidomonococcus sp., Lachnospiraceae bacterium COE1, Prevotella brevis ATCC19188, Smithella sp. SCADC, Moraxella bovoculi, Synergistes jonesii, Bacteroidetes oral taxon 274, Francisella tularensis, Leptospira inadai serovar Lyme str. 10, Acidomonococcus sp. crystal structure (5B43) S. mutans, S. agalactiae, S. equisimilis, S. sanguinis, S. pneumonia; C. jejuni, C. coli; N salsuginis, N. tergarcus; S. auricularis, S. carnosus; N meningitides, N gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Microgenomates, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, Porphyromonas macacae, Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM7374, Fructobacillus sp. EFB-N1, Weissella halotolerans, Pediococcus acidilactici , Lactobacillus curvatus , Streptococcus pyogenes, Lactobacillus versmoldensis, and Filifactor alocis ATCC35896. See, U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; 9,822,372; 9,840,713; U.S. patent application Ser. No. 13/842,859 (US 2014/0068797 A1); U.S. Pat. Nos. 9,260,723; 9,023,649; 9,834,791; 9,637,739; U.S. patent application Ser. No. 14/683,443 (US 2015/0240261 A1); U.S. patent application Ser. No. 14/743,764 (US 2015/0291961 A1); U.S. Pat. Nos. 9,790,490; 9,688,972; 9,580,701; 9,745,562; 9,816,081; 9,677,090; 9,738,687; U.S. application Ser. No. 15/632,222 (US 2017/0369879 A1); U.S. application Ser. No. 15/631,989; U.S. application Ser. No. 15/632,001; and U.S. Pat. No. 9,896,696, each of which is herein incorporated by reference.

In some embodiments, a Cas effector protein comprises one or more of the following activities:

a nickase activity, i.e., the ability to cleave a single strand of a nucleic acid molecule;

a double stranded nuclease activity, i.e., the ability to cleave both strands of a double stranded nucleic acid and create a double stranded break;

an endonuclease activity;

an exonuclease activity; and/or

a helicase activity, i.e., the ability to unwind the helical structure of a double stranded nucleic acid.

In aspects of the disclosure the term “guide nucleic acid” refers to a polynucleotide comprising 1) a guide sequence capable of hybridizing to a target sequence (referred to herein as a “targeting segment”) and 2) a scaffold sequence capable of interacting with (either alone or in combination with a tracrRNA molecule) a nucleic acid guided nuclease as described herein (referred to herein as a “scaffold segment”). A guide nucleic acid can be DNA. A guide nucleic acid can be RNA. A guide nucleic acid can comprise both DNA and RNA. A guide nucleic acid can comprise modified non-naturally occurring nucleotides. In cases where the guide nucleic acid comprises RNA, the RNA guide nucleic acid can be encoded by a DNA sequence on a polynucleotide molecule such as a plasmid, linear construct generated using the methods and compositions provided herein.

In some embodiments, the guide nucleic acids described herein are RNA guide nucleic acids (“guide RNAs” or “gRNAs”) and comprise a targeting segment and a scaffold segment. In some embodiments, the scaffold segment of a gRNA is comprised in one RNA molecule and the targeting segment is comprised in another separate RNA molecule. Such embodiments are referred to herein as “double-molecule gRNAs” or “two-molecule gRNA” or “dual gRNAs.” In some embodiments, the gRNA is a single RNA molecule and is referred to herein as a “single-guide RNA” or an “sgRNA.” The term “guide RNA” or “gRNA” is inclusive, referring both to two-molecule guide RNAs and sgRNAs.

In one embodiment, an assembly comprising a pair of homology arms with an insert polynucleotide located therebetween generated using the methods and compositions provided herein is a guide RNA (gRNA). In some cases, the methods provided herein are used to generate a library of gRNAs. Further to this embodiment, the gene editing methods provided herein can further comprise introducing a pool of donor DNA sequence (e.g., linear insert polynucleotides) into the host cell. The pool of donor DNA sequences can be introduced into the host cell prior to, along with or following the introduction of the library of gRNAs. Each of the donor DNA sequences in the pool of donor DNA sequences can comprise sequence complementary to a genomic locus targeted by the gRNAs. The pool of donor DNA sequences can comprise donor DNA sequences that target or bind the genomic loci targeted by each of the gRNAs in the library of gRNAs. The pool of donor DNA sequences can comprise donor DNA sequences that target or bind genomic loci targeted by a subset of gRNAs in the library of gRNAs.

The DNA-targeting segment of a gRNA comprises a nucleotide sequence that is complementary to a sequence in a target nucleic acid sequence. As such, the targeting segment of a gRNA interacts with a target nucleic acid in a sequence-specific manner via hybridization (i.e., base pairing), and the nucleotide sequence of the targeting segment determines the location within the target DNA that the gRNA will bind. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 75, or more nucleotides in length. In some embodiments, a guide sequence is less than about 75, 50, 45, 40, 35, 30, 25, 20 nucleotides in length. In aspects, the guide sequence is 10-30 nucleotides long. The guide sequence can be 15-20 nucleotides in length. The guide sequence can be 15 nucleotides in length. The guide sequence can be 16 nucleotides in length. The guide sequence can be 17 nucleotides in length. The guide sequence can be 18 nucleotides in length. The guide sequence can be 19 nucleotides in length. The guide sequence can be 20 nucleotides in length.

The scaffold segment of a guide RNA interacts with a one or more Cas effector proteins to form a ribonucleoprotein complex (referred to herein as a CRISPR-RNP or an RNP-complex). The guide RNA directs the bound polypeptide to a specific nucleotide sequence within a target nucleic acid sequence via the above-described targeting segment. The scaffold segment of a guide RNA comprises two stretches of nucleotides that are complementary to one another and which form a double stranded RNA duplex. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or encoded on the same polynucleotide. In some cases, the one or two sequence regions are comprised or encoded on separate polynucleotides. Optimal alignment may be determined by any suitable alignment algorithm, and may further account for secondary structures, such as self-complementarity within either the one or two sequence regions. In some embodiments, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some embodiments, at least one of the two sequence regions is about or more than about 5, 6, 7, 8, 9, 10, 11, 12, 13, 14, 15, 16, 17, 18, 19, 20, 25, 30, 40, 50, or more nucleotides in length.

A scaffold sequence of a subject gRNA can comprise a secondary structure. A secondary structure can comprise a pseudoknot region or stem-loop structure. In some examples, the compatibility of a guide nucleic acid and nucleic acid guided nuclease is at least partially determined by sequence within or adjacent to the secondary structure region of the guide RNA. In some cases, binding kinetics of a guide nucleic acid to a nucleic acid guided nuclease is determined in part by secondary structures within the scaffold sequence. In some cases, binding kinetics of a guide nucleic acid to a nucleic acid guided nuclease is determined in part by nucleic acid sequence with the scaffold sequence.

A compatible scaffold sequence for a gRNA-Cas effector protein combination can be found by scanning sequences adjacent to a native Cas nuclease locus. In other words, native Cas nucleases can be encoded on a genome within proximity to a corresponding compatible guide nucleic acid or scaffold sequence.

Nucleic acid guided nucleases can be compatible with guide nucleic acids that are not found within the nucleases endogenous host. Such orthogonal guide nucleic acids can be determined by empirical testing. Orthogonal guide nucleic acids can come from different bacterial species or be synthetic or otherwise engineered to be non-naturally occurring. Orthogonal guide nucleic acids that are compatible with a common nucleic acid-guided nuclease can comprise one or more common features. Common features can include sequence outside a pseudoknot region. Common features can include a pseudoknot region. Common features can include a primary sequence or secondary structure.

A guide nucleic acid can be engineered to target a desired target sequence by altering the guide sequence such that the guide sequence is complementary to the target sequence, thereby allowing hybridization between the guide sequence and the target sequence. A guide nucleic acid with an engineered guide sequence can be referred to as an engineered guide nucleic acid. Engineered guide nucleic acids are often non-naturally occurring and are not found in nature.

In some embodiments, the present disclosure provides a polynucleotide encoding a gRNA generated using the compositions and methods provided herein. In some embodiments, the composition comprising a pool of targeting polynucleotides and a pool of insert polynucleotides further comprises an expression vector or expression vectors comprising a gRNA-encoding nucleic acid or a pool of linear fragments each comprising a gRNA encoding nucleic acid.

In another embodiment, an assembly comprising a pair of homology arms with an insert polynucleotide located therebetween generated using the methods and compositions provided herein is a donor DNA sequence or a repair fragment. In some cases, the methods provided herein are used to generate a library of donor DNA sequences or a pool of linear insert polynucleotides that each serve as a donor DNA sequence or a repair fragment. The donor DNA sequence can be used in combination with a guide RNA (gRNA) or pool of gRNAs in a CRISPR method of gene editing using homology directed repair (HDR). The CRISPR complex can result in the strand breaks within the target gene(s) that can be repaired by using homology directed repair (HDR). HDR mediated repair can be facilitated by co-transforming the host cell with a donor DNA sequence generated using the methods and compositions provided herein. The donor DNA sequence can comprise a desired genetic perturbation or payload (e.g., deletion, insertion, and/or single nucleotide polymorphism) as well as targeting sequences derived from the homology arms. In this embodiment, the CRISPR complex cleaves the target gene specified by the one or more gRNAs. The donor DNA sequence can then be used as a template for the homologous recombination machinery to incorporate the desired genetic perturbation or payload into the host cell. The donor DNA can be single-stranded, double-stranded or a double-stranded plasmid. The donor DNA can lack a PAM sequence or comprise a scrambled, altered or non-functional PAM in order to prevent re-cleavage. In some cases, the donor DNA can contain a functional or non-altered PAM site. The mutated or edited sequence in the donor DNA (also flanked by the regions of homology) prevents re-cleavage by the CRISPR-complex after the mutation(s) has/have been incorporated into the genome. The gRNA or pool of gRNAs can be introduced into the host cell prior to, along with or following the introduction of the pool of linear insert polynucleotides. Each of the gRNAs in the pool of gRNAs can comprise sequence complementary to a genomic locus targeted by the homology arms in one or more of the linear insert polynucleotides present in the pool of linear insert polynucleotides. The pool of gRNAs can comprise gRNAs that target or bind the genomic loci targeted by each of the linear insert polynucleotides in the pool of linear insert polynucleotides. The pool of gRNAs can comprise gRNAs that target or bind genomic loci targeted by a subset of linear insert polynucleotides in the pool of linear insert polynucleotides.

### Host Cells

As provided herein, the libraries of nucleic acid constructs or linear insert polynucleotides generated using the compositions and/or methods provided herein can be used to edit or modify a genetic element (e.g., genome, cosmid or plasmid) of a host cell or engineer the host cell via introducing (e.g., transforming or transducing) one or more nucleic acid constructs or linear insert polynucleotides from the libraries generated using the methods and/or compositions herein into said host cell. The genomic engineering or editing methods can be applicable to any organism where desired traits can be identified in a population of genetic mutants. The organism can be a microorganism or higher eukaryotic organism.

Thus, as used herein, the term “microorganism” should be taken broadly. It includes, but is not limited to, the two prokaryotic domains, Bacteria and Archaea, as well as certain eukaryotic fungi and protists. However, in certain aspects, “higher” eukaryotic organisms such as insects, plants, and animals can be utilized in the methods taught herein.

Suitable host cells include, but are not limited to bacterial cells, algal cells, plant cells, fungal cells, insect cells, and mammalian cells. In one illustrative embodiment, suitable host cells include E. coli (e.g., strain W3110).

Other suitable host organisms of the present disclosure include microorganisms of the genus Corynebacterium. In some embodiments, preferred Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments, the preferred host of the present disclosure is C. glutamicum.

Suitable host strains of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

The term “Micrococcus glutamicus” has also been in use for C. glutamicum. Some representatives of the species C. efficiens have also been referred to as C. thermoaminogenes in the prior art, such as the strain FERM BP-1539, for example.

In some embodiments, the host cell of the present disclosure is a eukaryotic cell. Suitable eukaryotic host cells include, but are not limited to fungal cells, algal cells, insect cells, animal cells, and plant cells. Suitable fungal host cells include, but are not limited to Ascomycota, Basidiomycota, Deuteromycota, Zygomycota, Fungi imperfecti. Certain preferred fungal host cells include yeast cells and filamentous fungal cells. Suitable filamentous fungi host cells include, for example, any filamentous forms of the subdivision Eumycotina and Oomycota. (see, e.g., Hawksworth et al., In Ainsworth and Bisby's Dictionary of The Fungi, 8th. edition, 1995, CAB International, University Press, Cambridge, UK, which is incorporated herein by reference). Filamentous fungi are characterized by a vegetative mycelium with a cell wall composed of chitin, cellulose and other complex polysaccharides. The filamentous fungi host cells are morphologically distinct from yeast.

In certain illustrative, but non-limiting embodiments, the filamentous fungal host cell may be a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella, or teleomorphs, or anamorphs, and synonyms or taxonomic equivalents thereof.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In certain embodiments, the host cell is an algal such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, The rmosynechococcus, Thermococcus, Ureaplasma, Xanthomonas, Xylella, Yersinia, and Zymomonas. In some embodiments, the host cell is Corynebacterium glutamicum.

In some embodiments, the bacterial host strain is an industrial strain. Numerous bacterial industrial strains are known and suitable in the methods and compositions described herein.

In some embodiments, the bacterial host cell is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothennophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avennitihs, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

In some embodiments, the host cell will be an industrial Escherichia species (e.g., E. coli).

Suitable host strains of the E. coli species comprise: Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli O157:H7, E. coli O104:H4, Escherichia coli O121, Escherichia coli O104:H21, Escherichia coli K1, and Escherichia coli NC101. In some embodiments, the present disclosure teaches genomic engineering of E. coli K12, E. coli B, and E. coli C.

In some embodiments, the host cell can be E. coli strains NCTC 12757, NCTC 12779, NCTC 12790, NCTC 12796, NCTC 12811, ATCC11229, ATCC25922, ATCC8739, DSM 30083, BC 5849, BC 8265, BC 8267, BC 8268, BC 8270, BC 8271, BC 8272, BC 8273, BC 8276, BC 8277, BC 8278, BC 8279, BC 8312, BC 8317, BC 8319, BC 8320, BC 8321, BC 8322, BC 8326, BC 8327, BC 8331, BC 8335, BC 8338, BC 8341, BC 8344, BC 8345, BC 8346, BC 8347, BC 8348, BC 8863, and BC 8864.

In some embodiments, the present disclosure teaches host cells that can be verocytotoxigenic E. coli (VTEC), such as strains BC 4734 (O26:H11), BC 4735 (O157:H-), BC 4736, BC 4737 (n.d.), BC 4738 (O157:H7), BC 4945 (O26:H-), BC 4946 (O157:H7), BC 4947 (O111:H-), BC 4948 (O157:H), BC 4949 (O5), BC 5579 (O157:H7), BC 5580 (O157:H7), BC 5582 (O3:H), BC 5643 (O2:H5), BC 5644 (O128), BC 5645 (O55:H-), BC 5646 (O69:H-), BC 5647 (O101:H9), BC 5648 (O103:H2), BC 5850 (O22:H8), BC 5851 (O55:H-), BC 5852 (O48:H21), BC 5853 (O26:H11), BC 5854 (O157:H7), BC 5855 (O157:H-), BC 5856 (O26:H-), BC 5857 (O103:H2), BC 5858 (O26:H11), BC 7832, BC 7833 (O raw form:H-), BC 7834 (ONT:H-), BC 7835 (O103:H2), BC 7836 (O57:H-), BC 7837 (ONT:H-), BC 7838, BC 7839 (O128:H2), BC 7840 (O157:H-), BC 7841 (O23:H-), BC 7842 (O157:H-), BC 7843, BC 7844 (O157:H-), BC 7845 (O103:H2), BC 7846 (O26:H11), BC 7847 (O145:H-), BC 7848 (O157:H-), BC 7849 (O156:H47), BC 7850, BC 7851 (O157:H-), BC 7852 (O157:H-), BC 7853 (O5:H-), BC 7854 (O157:H7), BC 7855 (O157:H7), BC 7856 (O26:H-), BC 7857, BC 7858, BC 7859 (ONT:H-), BC 7860 (O129:H-), BC 7861, BC 7862 (O103:H2), BC 7863, BC 7864 (O raw form:H-), BC 7865, BC 7866 (O26:H-), BC 7867 (O raw form:H-), BC 7868, BC 7869 (ONT:H-), BC 7870 (O113:H-), BC 7871 (ONT:H-), BC 7872 (ONT:H-), BC 7873, BC 7874 (O raw form:H-), BC 7875 (O157:H-), BC 7876 (O111:H-), BC 7877 (O146:H21), BC 7878 (O145:H-), BC 7879 (O22:H8), BC 7880 (O raw form:H-), BC 7881 (O145:H-), BC 8275 (O157:H7), BC 8318 (O55:K-:H-), BC 8325 (O157:H7), and BC 8332 (ONT), BC 8333.

In some embodiments, the present disclosure teaches host cells that can be enteroinvasive E. coli (EIEC), such as strains BC 8246 (O152:K-:H-), BC 8247 (O124:K(72):H3), BC 8248 (O124), BC 8249 (O112), BC 8250 (O136:K(78):H-), BC 8251 (O124:H-), BC 8252 (O144:K-:H-), BC 8253 (O143:K:H-), BC 8254 (O143), BC 8255 (O112), BC 8256 (O28a.e), BC 8257 (O124:H-), BC 8258 (O143), BC 8259 (O167:K-:H5), BC 8260 (O128a.c.:H35), BC 8261 (O164), BC 8262 (O164:K-:H-), BC 8263 (O164), and BC 8264 (O124).

In some embodiments, the present disclosure teaches host cells that can be enterotoxigenic E. coli (ETEC), such as strains BC 5581 (O78:H11), BC 5583 (O2:K1), BC 8221 (O118), BC 8222 (O148:H-), BC 8223 (O111), BC 8224 (O110:H-), BC 8225 (O148), BC 8226 (O118), BC 8227 (O25:H42), BC 8229 (O6), BC 8231 (O153:H45), BC 8232 (O9), BC 8233 (O148), BC 8234 (O128), BC 8235 (O118), BC 8237 (O111), BC 8238 (O110:H17), BC 8240 (O148), BC 8241 (O6H16), BC 8243 (O153), BC 8244 (O15:H-), BC 8245 (O20), BC 8269 (O125a. c:H-), BC 8313 (O6:H6), BC 8315 (O153:H-), BC 8329, BC 8334 (O118:H12), and BC 8339.

In some embodiments, the present disclosure teaches host cells that can be enteropathogenic E. coli (EPEC), such as strains BC 7567 (O86), BC 7568 (O128), BC 7571 (O114), BC 7572 (O119), BC 7573 (O125), BC 7574 (O124), BC 7576 (O127a), BC 7577 (O126), BC 7578 (O142), BC 7579 (O26), BC 7580 (OK26), BC 7581 (O142), BC 7582 (O55), BC 7583 (O158), BC 7584 (O-), BC 7585 (O-), BC 7586 (O-), BC 8330, BC 8550 (O26), BC 8551 (O55), BC 8552 (O158), BC 8553 (O26), BC 8554 (O158), BC 8555 (O86), BC 8556 (O128), BC 8557 (OK26), BC 8558 (O55), BC 8560 (O158), BC 8561 (O158), BC 8562 (O114), BC 8563 (O86), BC 8564 (O128), BC 8565 (O158), BC 8566 (O158), BC 8567 (O158), BC 8568 (O111), BC 8569 (O128), BC 8570 (O114), BC 8571 (O128), BC 8572 (O128), BC 8573 (O158), BC 8574 (O158), BC 8575 (O158), BC 8576 (O158), BC 8577 (O158), BC 8578 (O158), BC 8581 (O158), BC 8583 (O128), BC 8584 (O158), BC 8585 (O128), BC 8586 (O158), BC 8588 (O26), BC 8589 (O86), BC 8590 (O127), BC 8591 (O128), BC 8592 (O114), BC 8593 (O114), BC 8594 (O114), BC 8595 (O125), BC 8596 (O158), BC 8597 (O26), BC 8598 (O26), BC 8599 (O158), BC 8605 (O158), BC 8606 (O158), BC 8607 (O158), BC 8608 (O128), BC 8609 (O55), BC 8610 (O114), BC 8615 (O158), BC 8616 (O128), BC 8617 (O26), BC 8618 (O86), BC 8619, BC 8620, BC 8621, BC 8622, BC 8623, BC 8624 (O158), and BC 8625 (O158).

In some embodiments, the present disclosure also teaches host cells that can be Shigella organisms, including Shigella flexneri, Shigella dysenteriae, Shigella boydii, and Shigella sonnei.

The present disclosure is also suitable for use with a variety of animal cell types, including mammalian cells, for example, human (including 293, WI38, PER.C6 and Bowes melanoma cells), mouse (including 3T3, NS0, NS1, Sp2/0), hamster (CHO, BHK), monkey (COS, FRhL, Vero), and hybridoma cell lines.

In various embodiments, strains that may be used in the practice of the disclosure including both prokaryotic and eukaryotic strains, are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In some embodiments, the methods of the present disclosure are also applicable to multi-cellular organisms. For example, the platform could be used for improving the performance of crops. The organisms can comprise a plurality of plants such as Gramineae, Fetucoideae, Poacoideae, Agrostis, Phleum, Dactylis, Sorgum, Setaria, Zea, Oryza, Triticum, Secale, Avena, Hordeum, Saccharum, Poa, Festuca, Stenotaphrum, Cynodon, Coix, Olyreae, Phareae, Compositae or Leguminosae. For example, the plants can be corn, rice, soybean, cotton, wheat, rye, oats, barley, pea, beans, lentil, peanut, yam bean, cowpeas, velvet beans, clover, alfalfa, lupine, vetch, lotus, sweet clover, wisteria, sweet pea, sorghum, millet, sunflower, canola or the like. Similarly, the organisms can include a plurality of animals such as non-human mammals, fish, insects, or the like.

### Transformation of Host Cells

In some embodiments, the constructs generated by the methods of the present disclosure may be introduced into the host cells using any of a variety of techniques, including transformation, transfection, transduction, viral infection, gene guns, or Ti-mediated gene transfer. Particular methods include calcium chloride-mediated transformation, calcium phosphate transfection, DEAE-Dextran mediated transfection, lipofection, or electroporation (Davis, L., Dibner, M., Battey, I., 1986 “Basic Methods in Molecular Biology”). Other methods of transformation include for example, lithium acetate transformation and electroporation See, e.g., Gietz et al., Nucleic Acids Res. 27:69-74 (1992); Ito et al., J. Bacterol. 153:163-168 (1983); and Becker and Guarente, Methods in Enzymology 194:182-187 (1991). In some embodiments, transformed host cells are referred to as recombinant host strains.

### Automation

In one embodiment, the compositions and methods provided herein are incorporated into a high-throughput (HTP) method for genetic engineering of a host cell. In some cases, the HTP method is automated. The automated HTP method can utilize robotic machines (i.e., liquid handlers, multi-tip pipettors, etc.). The robotic machines can be connected via one or more computers comprising one or more memories. The one or more memories can comprise or implement software programs that can instruct the robotic machines to conduct any of the methods provided herein. In another embodiment, the methods provided herein can be a molecular tool that is part of the suite of HTP molecular tool sets described in PCT/US18/36360, PCT/US18/36333 or WO 2017/100377, each of which is herein incorporated by reference, for all purposes, to create HTP genetic design libraries, which are derived from, inter alia, scientific insight and iterative pattern recognition. The compositions and methods provided herein can be used to generate libraries for use in high-throughput methods such as those described in PCT/US18/36360, PCT/US18/36333 or WO 2017/100377. Examples of libraries that can be generated using the methods provided herein can include, but are not limited to promoter ladders, terminator ladders, solubility tag ladders or degradation tag ladders. Examples of high-throughput genomic engineering methods that can utilize the compositions and methods provided herein can include, but are not limited to, promoter swapping, terminator (stop) swapping, solubility tag swapping, degradation tag swapping or SNP swapping as described in PCT/US18/36360, PCT/US18/36333 or WO 2017/100377. The high-throughput methods can be automated and/or utilize robotics and liquid handling platforms (e.g., plate robotics platform and liquid handling machines known in the art. The high-throughput methods can utilize multi-well plates such as, for example microtiter plates.

In some embodiments, the automated methods of the disclosure comprise a robotic system. The systems outlined herein are generally directed to the use of 96- or 384-well microtiter plates, but as will be appreciated by those in the art, any number of different plates or configurations may be used. In addition, any or all of the steps outlined herein may be automated; thus, for example, the systems may be completely or partially automated. The robotic systems compatible with the methods and compositions provided herein can be those described in PCT/US18/36360, PCT/US18/36333 or WO 2017/100377.

### Kits

Also provided by the present disclosure are kits for practicing the methods for generating nucleic acid assemblies or libraries derived therefrom as described above. The kit can comprise a mixture containing all of the reagents necessary for assembling ssDNA molecules (e.g., oligonucleotides) or dsDNA molecules. In certain embodiments, a subject kit may contain: (i) a first pool of targeting polynucleotides, (ii) a second pool of insert polynucleotides, wherein each targeting polynucleotide from the first pool comprises, from 5′ to 3′, a first assembly overlap sequence comprising sequence that binds to (e.g., via complementarity) a distal end of an insert polynucleotide from the second pool, a first homology arm, a linearization sequence, a second homology arm and a second assembly overlap sequence comprising sequence that binds to (e.g., via complementarity) a proximal end of the insert polynucleotide and (iii) optionally, a pool of reverse primers, wherein, for each insert polynucleotide from the second pool, the reverse primer comprises sequence that binds to (e.g., via complementarity) the distal end of the insert polynucleotide. In some cases, the kit includes a positive control.

In one embodiment, the kits provided herein further comprise a 5′-3′ exonuclease, and a strand-displacing polymerase. In another embodiment, the kits provided herein further comprise a 5′-3′ exonuclease, a ligase and a strand-displacing polymerase. In a still further embodiment, the kits provided herein comprise a single-stranded (ss) binding protein. The ss binding protein can be an extreme thermostable single-stranded DNA binding protein (ET SSB), E. coli recA, T7 gene 2.5 product, phage lambda RedB or Rac prophage RecT.

In a separate embodiment, the kits provided herein further comprise a 5′ to 3′ exonuclease that lacks 3′ exonuclease activity, a crowding agent, a thermostable non-strand-displacing DNA polymerase with 3′ exonuclease activity, or a mixture of said DNA polymerase with a second DNA polymerase that lacks 3′ exonuclease activity, and an isolated thermostable ligase, in appropriate amounts. The crowding agent can PEG, dextran or Ficoll. For example, the kit may contain T5 exonuclease, PEG, PHUSION®. DNA polymerase, and Taq ligase. In another example, the kit comprises: Exonuclease III, PEG, AMPLITAQ GOLD® DNA polymerase, and Taq ligase.

In a separate embodiment, the kits provided herein further comprises one or more Type IIS restriction enzymes and a T4 DNA ligase.

Any of the kits provided herein may also contain other reagents described above and below that may be employed in the method, e.g., a mismatch repair enzyme such as mutHLS, cel-1 nuclease, T7 endo 1, uvrD, T4 EndoVII, E. coli EndoV, a buffer, dNTPs, plasmids into which to insert the synthon and/or competent cells to receive the plasmids, controls etc., depending on how the method is going to be implemented.

The components of the kit may be combined in one container, or each component may be in its own container. For example, the components of the kit may be combined in a single reaction tube or in one or more different reaction tubes.

In addition to above-mentioned components, the subject kit further includes instructions for using the components of the kit to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded.

Compositions, kits and methods for assembling pairs of targeting polynucleotides in a first pool and insert polynucleotides in a second pool as described herein result in a product that is a dsDNA that can serve as a template for PCR, RCA or a variety of other molecular biology applications including linearization and direct transformation or transfection of a competent prokaryotic or eukaryotic host cell.

### EXAMPLES

The present disclosure is further illustrated by reference to the following Examples. However, it should be noted that these Examples, like the embodiments described above, are illustrative and are not to be construed as restricting the scope of the disclosure in any way.

### Example 1—Proof of Principle of Method for Double-Crossover Editing of S. cerevisiae with Pooled Circular-Permuted Fragments

### Objective

The goal of the experiments described in this Example is to demonstrate that DNA pools created using the methods provided herein (e.g., FIG. 1A-1C) can be used to edit S. cerevisiae via double-crossover homologous recombination. To do this, a host S. cerevisiae strain was transformed with a 54-member library targeting a single payload to 54 distinct loci in the host cell genome.

### Materials and Methods

Preparation of DNA for Transformation:

As shown in FIGS. 2A-2C, a payload consisting of (5′→3′) a primer-binding site, a URA selection marker flanked by 150 bp direct repeats, and a native promoter sequence was amplified with a pooled forward primer and single reverse primer. This payload was carried on a pUC19-based vector. Each oligonucleotide in the forward primer pool consisted of (5′→3′) 20 bp homologous to the distal end of the payload, one of 54 distinct right homology sequences, an I-Scel recognition sequence, one 54 distinct left homology sequences, and one of 54 distinct barcode sequences flanked by universal amplification sequences. The 3′ universal amplification sequence was identical to the 5′ primer binding site on the payload. The forward primer pool was synthesized by IDT as an O-pool. The common reverse primer was designed to bind to the distal end of the payload. 18 cycles of PCR were performed to create the pooled product; the number of cycles was limited to limit the bias amongst sequences in the pooled product. The amplified product was treated with DpnI (NEB; manufacturer's protocol) for one hour to remove plasmid template and cleaned up with magnetic beads (AxyPrep®; manufacturer's protocol). This pool was then circularized in a HiFi® reaction (NEB master mix; 0.2 picomoles pooled product in a 20 μL total reaction volume). The circularized material was cleaned up using magnetic beads and amplified using rolling-circle amplification (Lucigen NxGen phi29 polymerase; manufacturer's whole-genome amplification protocol). The amplified material was treated with I-SceI (NEB; manufacturer's protocol) for one hour to produce linear monomers.

Preparation of Host Cells for Transformation:

S. cerevisiae cen.pk was streaked on a YPD-agar plate to isolate single colonies. A single colony was picked into 50 mL of YPD and grown at 30° C. with shaking at 250 RPM for 48 hours. This culture was used to inoculate a 100 mL YPD main culture at OD 0.25 (600 nm, 1 cm path). The main culture was grown at 30° C. with shaking at 250 RPM for approx. 5 hours until it reached an OD of 1. At this point the cells were washed twice in lithium acetate by spinning down at 4,000 ×g for 5 min, decanting the supernatant, and mixing with 50 mL of 100 mM LiOAc. After the second decanting, the cells were resuspended in 25 mL of 100 mM LiOAc and incubated at 30° C. for 30 min with shaking at 160 rpm. The cells were then spun at 6000 rpm for 5 min and resuspended in 100 mM LiOAc to a final OD of 100 (approx. 1.4 mL). The washed and concentrated cells were stored in a Parafilm-sealed tube at 4° C. overnight until use.

Heat Shock Transformation:

Transformation reactions contained 100 μL of the prepared competent yeast, 10 μL of freshly boiled salmon sperm DNA at 10 mg/mL, 600 μL of 40% PEG-3350 in 100 mM LiOAc, and 1-3 μg DNA. These were incubated at 30° C. with shaking at 160 rpm for 30 min. 70 μL of DMSO was then added and mixed immediately. The cells were then heat shocked at 42° C. for 25 min. To recover, the samples were cooled on ice for 3 min, centrifuged at 4,000×gfor 4 min, resuspended in 1 mL YPD and shaken gently at 30° C. for 2 hours. After recovery, the samples were centrifuged at 4,000×gfor 4 min, resuspended in 800 μL of selective medium (SD-Ura), and plated onto selective agar medium.

Edit Confirmation:

Since edits within the pool were directed to distinct loci distributed through the genome, the presence of edited material in a cell was first determined using the barcodes on each construct. Individual colonies were picked into liquid media, grown to saturation, and lysed. Primers designed to bind to the universal sequences flanking the barcodes were used to amplify the barcode sequences from each sample; the amplicons were indexed, pooled, and sequenced using an Illumina 2×150 MiSeq kit. The unique barcode sequences were queried within the raw reads to identify the edit present in each sample. Once the edit was identified, the edit was confirmed to be clean and at the intended locus by sequencing amplicons created using locus-specific primers for the junction between the edit and genomic context.

### Results

FIG. 2D shows the distribution of genotypes recovered after barcoding. Each bar is a unique genotype corresponding to one of the members of the transformed pool. Twenty-five (25) genotypes were recovered from a total of thirty-six (36) samples analyzed. FIG. 2E shows results from locus-specific sequencing confirmation of barcoded strains. Data was obtained for thirty (30) samples representing twenty-one (21) unique strains (genotypes), of which eight (8) samples representing seven (7) strains had a clean, on-target edit. “Parental sequence” could indicate an ectopic integration, while mixed population indicated that the samples had a combination of parental and on-target genotypes.

### Example 2—Proof of Principle of Method for CRISPR Mediated Homology Directed Repair of S. cerevisiae using Pooled Circular-Permuted Fragments as Repair Fragments

### Objective

The goal of the experiments described in this Example is to demonstrate that DNA pools created using the methods provided herein (e.g., FIG. 1A-1C) can be used to edit S. cerevisiae via CRISPR-mediated Homology Directed Repair (HDR). To do this, a host S. cerevisiae strain was transformed with payloads targeting each of three (3) loci in the genome using CRISPR/Cas9 mediated homologous recombination.

### Materials and Methods

Preparation of DNA for Transformation:

As shown in FIGS. 3A-3C, payloads consisting of a primer binding site and native promoter sequence were amplified with a pooled forward primer and a single reverse primer. The payloads were carried on a pUC19-based plasmid. Each oligonucleotide in the forward primer pool consisted of (5′→3′) 20 bp homologous to the distal end of the payload, one (1) of nine (9) distinct right homology arms, one (1) of nine (9) pairs of primer binding sites, one (1) of nine (9) matching left homology arms, and one (1) of nine (9) barcode sequences flanked by universal amplification sequences. The 3′ universal amplification sequence was identical to the 5′ primer binding site on the payload. The forward primer pool was synthesized by Integrated DNA Technologies (IDT; Coralville, Iowa) as an O-pool. The common reverse primer was designed to bind to the distal end of the payload. A touchdown PCR was completed with annealing temperatures ramping from 68° C. to 58° C. over 12 cycles, followed by 22 cycles annealing at 58° C. The PCR amplified products were purified on Clean and Concentrate columns following the manufacturer's instructions (Zymo Research). The purified amplicons were then circularized in a HiFi® reaction (NEB; manufacturer's protocol) with 10 ng of PCR product per reaction. The pools of circularized molecule were then used as the template in PCR amplifications to amplify a payload with the homology arms and barcode for a specific locus with primers binding to the primer binding sites between the two homology arms on each circularized molecule. These amplicons were then purified using magnetic beads (AxyPrep®; manufacturer's protocol) and quantified for use as repair templates or donor DNA fragments in CRISPR/Cas9 mediated genome editing using HDR.

Preparation of Host Cells for Transformation:

S. cerevisiae cen.pk was streaked on a YPD-agar plate to isolate single colonies. A single colony was picked into 10 mL of YPD and grown at 30° C. with shaking at 250 RPM for 24 hours. This culture was used to inoculate a 500 mL YPD main culture. The main culture was grown at 30° C. with shaking at 250 RPM for approx. 16 hours until it reached an OD of 0.8. At this point the cells were washed twice in lithium acetate by spinning down at 4,000×gfor 5 min, decanting the supernatant, and mixing with 50 mL of 100 mM LiOAc. After the second decanting, the cells were resuspended in 25 mL of 100 mM LiOAc. The cells were then spun at 6000 rpm for 5 min and resuspended in 100 mM LiOAc to a final OD of 100 (approx. 1.4 mL).

Heat Shock Transformation:

Transformation reactions contained 15 μL of the prepared competent yeast, 16 μL of DNA containing: 70 ng of plasmid backbone comprising a Nourseothricin resistance gene, 90 ng of gRNA expression cassette and 500 ng of repair template for each targeted locus, 119 ul of transformation mix containing 100 ul of 50%PEG 3350, 15 ul of 1M LiOAc and 4 ul of 10 mg/mL freshly boiled salmon sperm DNA. The cells were incubated at 30° C. for 30 min, immediately followed by a heat shock at 42° C. for 45 minutes. After heat shock the cells were washed in 1 mL of YPD, then resuspended in YPD and allowed to recover at 30° C. for 3 hours with agitation. Transformants were then selected on YPD agar containing 100 ug/mL Nourseothricin Sulfate.

Edit Confirmation:

In each transformation, edits were confirmed via structural PCR of the target locus with primers flanking the site. Samples containing the native for ATR1 produced a PCR product size of 482 bp while samples edited for ATR1 produced an 832 bp PCR product.

### Results

FIG. 3D shows results from locus specific (ATR1 gene locus) sequencing confirmation of barcoded strains. Data was obtained for 48 samples, of which four (4) samples had a clean, on-target edit. “Parental sequence” could indicate an ectopic integration, while mixed population indicated that the samples had the parental, ectopic and on-target edits.

### Example 3—Additional Application of Circular Permutation for the Creation of Payloads for Saccharomyces cerevisiae Transformation

### Objective

The goal of the experiments described in this Example is to demonstrate that the circular permutation methods described throughout this application can be used to generate pools of nucleic acid (e.g., DNA) inserts or payloads that are generally difficult to produce via processes such as DNA synthesis such as, for example, payloads that have a high AT content.

### Materials and Methods

Preparation of DNA for Transformation:

As shown in FIG. 4, gBlocks comprising a pair of homology arms comprising sequence complementary to a genomic locus, a linearization sequence located immediately between the pair of homology arms, a recognition sequence for the Bbsl restriction enzyme (i.e., Type IIS restriction enzyme) on opposing ends of each gBlock®, and a 4-bp site on opposing ends of each gBlock® that allowed for ligation onto an intended payload promoter sequence following digestion with the Bbsl restriction enzyme were designed and generated. Additionally, as shown in FIG. 4, a payload promoter sequence was amplified from the S. cerevisiae genome using a primer pair designed to amplify the payload promoter sequence and to add recognition sequence for the BbsI restriction enzyme to opposing ends of the payload promoter sequence. The payload promoter sequence amplicon is shown as the payload PCR product in FIG. 4. The primer pair comprised a forward and a reverse primer that comprised sequence complementary to the payload promoter sequence or reverse complement thereof and tails comprising non-complementary sequence that comprised recognition sequences for the Bbsl restriction enzyme. In particular, each primer in the primer pair comprised, from 5′ to 3′, 8-bp of random sequence, a recognition sequence for the BbsI restriction enzyme, a 4-bp site that allowed for ligation onto an intended targeting polynucleotide (i.e., homology arm containing-gBlock® in FIG. 4) and the sequence complementary to the promoter payload sequence or reverse complement thereof (i.e., the priming site onto the promoter payload sequence). It should be noted that the gBlocks further comprised additional payload sequence (barcodes and gene coding sequence modifications) that flanks one or both of the homology arms.

Prior to assembly, both the gBlocks (i.e., targeting polynucleotides) and payload PCR product (i.e., insert polynucleotides) were amplified using primer pairs comprising forward and reverse primers that bound the ends of either the gBlocks or payload PCR product, respectively. Both the gBlock and payload PCR product amplicons were then purified and assembled using BbsI-based Golden Gate Assembly®. The circular assemblies generated via the Golden Gate Assembly® reaction then served as templates for the final payload amplification (i.e., PCR) reaction using a primer pair comprising primers that bound the linearization sequence as shown in FIG. 4 and extended in opposite directions from the linearization sequence to produce linear monomers. The desired linear payload PCR product was then gel-purified and used for the transformation of Saccharomyces cerevisiae.

Four payloads were prepared following this process and were each used as one payload in duplexed CRISPR/Cas9 mediated genome editing via HDR of Saccharomyces cerevisiae as described in Example 2 and below. In the CRISPR/Cas9 mediated genome editing, for each of the four payloads, the linear payload PCR served as the repair fragment and was co-transformed with a gRNA cassette directed to the genome locus targeted by the homology arms for the respective payload.

Preparation of Host Cells for Transformation:

S. cerevisiae cen.pk was streaked on a YPD-agar plate to isolate single colonies. A single colony was picked into 10 mL of YPD and grown at 30° C. with shaking at 250 RPM for 24 hours. This culture was used to inoculate a 500 mL YPD main culture. The main culture was grown at 30° C. with shaking at 250 RPM for approx. 16 hours until it reached an OD of 0.8. At this point the cells were washed twice in lithium acetate by spinning down at 4,000×gfor 5 min, decanting the supernatant, and mixing with 50 mL of 100 mM LiOAc. After the second decanting, the cells were resuspended in 25 mL of 100 mM LiOAc. The cells were then spun at 6000 rpm for 5 minutes and resuspended to a final OD of 100 (approx. 1.4 mL).

Heat Shock Transformation:

Transformation reactions contained 15 μl of the prepared competent yeast, 16 μl of DNA containing: 70 ng of plasmid backbone comprising a Nourseothricin resistance gene, 90 ng of gRNA expression cassette and 500 ng of repair template for each targeted locus, 119 ul of transformation mix containing 100 ul of 50%PEG 3350, 15 ul of 1M LiOAc and 4 ul of 10 mg/mL freshly boiled salmon sperm DNA. The cells were incubated at 30° C. for 30 min, immediately followed by a heat shock at 42° C. for 45 minutes. After heat shock the cells were washed in 1 mL of YPD, then resuspended in YPD and allowed to recover at 30° C. for 3 hours with agitation. Transformants were then selected on YPD agar containing 100 ug/mL Nourseothricin Sulfate.

### Results

Payloads prepared using the circularization and linearization process performed comparably to fully synthesized payloads with overall colony QC pass rates for transformations using one circularized and linearized payload of 17.1%, 16.4%, 8% and 14.8% respectively.

### Numbered Embodiments of the Disclosure

Other subject matter contemplated by the present disclosure is set out in the following numbered embodiments:

1. A method for genetically editing a host cell, the method comprising:
(a) assembling a pool of insert polynucleotides and a pool of targeting polynucleotides into a pool of circular molecules, wherein each circular molecule from the pool of circular molecules comprises one or more payload sequences flanked by a first homology arm 5′ to the one or more payload sequences and a second homology arm 3′ to the one or more payload sequences and a linearization sequence that is located between both the first and second homology arms;
(b) linearizing each of the circular molecules from the pool of circular molecules via the linearization sequence , thereby generating a pool of linear insert polynucleotides, wherein each linear insert polynucleotide in the pool comprises from 5′ to 3′ a first homology arm, one or more payload sequences and a second homology arm, wherein the first homology arm and the second homology arm comprise sequence complementary to a genomic locus in a host cell; and
(c) introducing the pool of linear insert polynucleotides into the host cell, thereby genetically editing the host cell.

2. The method of embodiment 1, wherein the assembling of step (a) comprises:

(i) providing a pool of reverse primers along with the pool of insert polynucleotides and the pool of targeting polynucleotides, wherein the pool of targeting polynucleotides are forward primers, thereby generating a mixture comprising the pool of insert polynucleotides, the pool of forward primers and the pool of reverse primers, wherein, for each insert polynucleotide, the mixture comprises at least one forward primer from the pool of forward primers and a reverse primer from the pool of reverse primers, wherein the at least one forward primer comprises from 5′ to 3′, a first assembly overlap sequence comprising sequence complementary to a distal or 3′ end of the insert polynucleotide, the first homology arm, the linearization sequence, the second homology arm and a second assembly overlap sequence comprising sequence complementary to a reverse complement of a proximal or 5′ end of the insert polynucleotide, and wherein the reverse primer comprises sequence complementary to the distal or 3′ end of the insert polynucleotide;
(ii) performing a polymerase chain reaction (PCR) on the mixture, wherein, for each insert polynucleotide, the PCR generates a PCR product comprising from 5′ to 3′, the first assembly overlap sequence, the first homology arm, the linearization sequence, the second homology arm and the one or more payload sequences; and
(iii) circularizing the PCR products from step (ii) via an assembly method selected from the group consisting of splicing and overlap-extension PCR (SOE-PCR), Uracil-specific excision reagent (USER) cloning, restriction-ligation, scarless restriction-ligation, blunt-end ligation, an overlap based assembly method and recombination-based method, or any other enzymatic or chemical method of joining two DNA molecules.

3. The method of embodiment 1, wherein the assembling of step (a) comprises directly performing an assembly method on a mixture comprising the pool of insert polynucleotides and the pool of targeting polynucleotides, wherein, for each insert polynucleotide, the mixture comprises at least one targeting polynucleotide from the pool of targeting polynucleotides, wherein the at least one targeting polynucleotide comprises from 5′ to 3′, a first assembly overlap sequence comprising sequence complementary to a distal or 3′ end of the insert polynucleotide, the first homology arm, the linearization sequence, the second homology arm and a second assembly overlap sequence comprising sequence complementary to a reverse complement of a proximal or 5′ end of the insert polynucleotide, and wherein the assembly method is selected from selected from the group consisting of splicing and overlap-extension PCR (SOE-PCR), Uracil-specific excision reagent (USER) cloning, restriction-ligation, blunt-end ligation, overlap based assembly method and recombination-based method, or any other enzymatic or chemical method of joining two DNA molecules.

4. The method of embodiment 3, wherein the assembling method is an overlap based assembly method utilizing a Type IIS restriction enzyme and a ligase, wherein each insert polynucleotide in the pool of insert polynucleotides comprises a recognition sequence for the Type IIS restriction enzyme on both the insert polynucleotide's proximal or 5′ end and distal or 3′ end which upon digestion with the Type IIS restriction enzyme generates a proximal overhang and distal overhang, respectively, and wherein, for each insert polynucleotide, the mixture comprises at least one targeting polynucleotide from the pool of targeting polynucleotides, wherein the first assembly overlap sequence and the second assembly overlap sequence of the targeting polynucleotide each comprise the recognition sequence for the Type IIS restriction enzyme which upon digestion with the Type IIS restriction enzyme generates an overhang in the first assembly overlap sequence compatible with the distal overhang of the insert polynucleotide as well as an overhang in the second assembly overlap sequence compatible with the proximal overhang of the insert polynucleotide.

5. The method of embodiment 4, wherein the Type IIS restriction enzyme is a Type IIS restriction enzyme that generates a four-base overhang.

6. The method of embodiment 5, wherein the Type IIS restriction enzyme is selected from the group consisting of Bsal, Bbsl, BsmBI and Esp3I.

7. The method of any one of embodiments 4-6, wherein the ligase is a T4 DNA ligase.

8. The method of any one or embodiments 3-7, wherein each targeting polynucleotide in the pool of targeting polynucleotides is subjected to a primer extension reaction using a reverse primer comprising sequence that binds to the second assembly overlap sequence, thereby generating a double-stranded (ds) targeted polynucleotide.

9. The method of embodiment 8, wherein the top or sense strand of each ds targeting polynucleotide comprises, from 5′ to 3′, the first assembly overlap sequence comprising sequence complementary to the distal or 3′ end of the insert polynucleotide, the first homology arm, the linearization sequence, the second homology arm and the second assembly overlap sequence comprising sequence complementary to the reverse complement of the proximal or 3′ end of the insert polynucleotide.

10. The method of any one of the above embodiments, wherein the linearizing of step (b) comprises rolling circle amplification (RCA) of each circular molecule from the pool of circular molecules, wherein the RCA of each circular molecule produces a concatenated linear product comprising repeated units each separated by the linearization sequence, wherein each of the repeated units comprises the insert polynucleotide flanked upstream by the first homology arm and downstream by the second homology arm, wherein the insert polynucleotides are released from the concatenated linear product via the linearization sequence present between each repeated unit, thereby generating the pool of linear insert polynucleotides.

11. The method of any one of the above embodiments, wherein the linearization sequence comprises one or more recognition sequences for one or more site-specific nucleases.

12. The method of embodiment 11, wherein the linearizing comprises digesting the one or more recognition sequences with one or more site-specific nuclease(s) that recognize the one or more site-specific nuclease recognition sequence(s).

13. The method of embodiment 11 or 12, wherein the one or more site-specific nuclease(s) recognition sequence are for one or more of Type I restriction endonuclease(s), Type IIS restriction endonuclease(s), meganuclease, RNA-guided nuclease(s), DNA-guided nuclease(s), zinc-finger nuclease(s), TALEN(s) or nicking enzyme(s).

14. The method of any one of embodiments 1-10, wherein the linearization sequence comprises one or more primer binding sites that are common to each targeting polynucleotide in the pool of targeting polynucleotides.

15. The method of embodiment 14, wherein the linearizing of step (b) comprises performing a PCR using a primer pair directed to one of the one or more primer binding sites located within the linearization sequence.

16. The method of embodiment 14 or 15, wherein at least one of the one or more primer binding sites in the targeting polynucleotide is common to at least one of the one or more primer binding sites in each other targeting polynucleotide in the pool of targeting polynucleotides.

17. The method of embodiment 16, wherein the primer pair directed to one of the one or more primer binding sites located within the linearization sequence in step (b) is directed to the primer binding site common to each targeting polynucleotide in the pool of targeting polynucleotides.

18. The method of embodiment 14 or 15, wherein at least one of the one or more primer binding sites in the targeting polynucleotide is not found in any of the one or more primer binding sites in each other targeting polynucleotide in the pool of targeting polynucleotides.

19. The method of embodiment 18, wherein the primer pair directed to one of the one or more primer binding sites located within the linearization sequence in step (b) is directed to the primer binding site not found in any of the one or more primer binding sites in each other targeting polynucleotide in the pool of targeting polynucleotides.

20. The method of embodiment 14 or 15, wherein at least one of the one or more primer binding sites in the targeting polynucleotide is common to at least one of the one or more primer binding sites in a subset of other targeting polynucleotides in the pool of targeting polynucleotides.

21. The method of embodiment 20, wherein the primer pair directed to one of the one or more primer binding sites located within the linearization sequence in step (b) is directed to the primer binding site common to the subset of other targeting polynucleotides in the pool of targeting polynucleotides.

22. The method of any one of embodiments 2-21, wherein the first assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the distal or 3′ end of the insert polynucleotide.

23. The method of any one of embodiments 2-22, wherein the second assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the reverse complement of the proximal or 5′ end of the insert polynucleotide.

24. The method of any one of embodiments 2-23, wherein the distal or 3′ end of the insert polynucleotide to which the first assembly overlap sequence comprises sequence complementary thereto is found within one of the one or more payload sequences.

25. The method of any one of embodiments 2-23, wherein the distal or 3′ end of the insert polynucleotide to which the first assembly overlap sequence comprises sequence complementary thereto is found downstream of the one or more payload sequences.

26. The method of any one of embodiments 2-25, wherein the proximal or 5′ end of the insert polynucleotide to which the second assembly overlap sequence comprises sequence complementary to the reverse complement thereof is found within one of the one or more payload sequences.

27. The method of any one of embodiments 2-25, wherein the proximal or 5′ end of the insert polynucleotide to which the second assembly overlap sequence comprises sequence complementary to the reverse complement thereof is found upstream of the one or more payload sequences.

28. The method of any one of embodiments 2-27, wherein the distal or 3′ end of the insert polynucleotide to which the reverse primer comprises sequence complementary thereto is found within one of the one or more payload sequences.

29. The method of any one of embodiments 2-27, wherein the distal or 3′ end of the insert polynucleotide to which the reverse primer comprises sequence complementary thereto is found downstream of the one or more payload sequences.

30. The method of any one of the above embodiments, wherein each insert polynucleotide is present on a plasmid.

31. The method of any one of embodiments 1-29, wherein each insert polynucleotide is a linear fragment of nucleic acid.

32. The method of embodiment 31, wherein each linear insert polynucleotide is a gBlock.

33. The method of embodiment 31, wherein each insert polynucleotide is single-stranded or double-stranded.

34. The method of any one of the above embodiments, wherein each payload sequence is selected from the group consisting of whole or portions of promoters, genes, regulatory sequences, nucleic acid sequence encoding degrons, nucleic acid sequence encoding solubility tags, terminators, unique identifier sequence, and combinations thereof.

35. The method of any one of the above embodiments, wherein each payload sequence and/or targeting polynucleotide further comprises a barcode sequence.

36. The method of embodiment 35, wherein the barcode sequence comprises a sequence unique to each combination of payload sequence and first and second homology arms flanked by sequence universal to the barcode sequence present in each other payload sequence.

37. The method of embodiment 36, wherein the sequence universal to the barcode sequence present in each other payload sequence is used for amplifying or sequencing the unique sequence in each barcode.

38. The method of any one of the above embodiments, wherein the insert polynucleotide further comprises sequence for a selectable marker.

39. The method of embodiment 38, wherein the sequence for the selectable marker is flanked by direct repeat sequences that serve to facilitate looping out of the sequence for the selectable marker.

40. The method of embodiment 38 or 39, wherein the selectable marker is selected from the group consisting of an antibiotic resistance gene, an auxotrophic marker, a colorimetric marker, a gene for a reporter protein and a directional marker.

41. The method of any one of the above embodiments, wherein the first and second homology arms on each circular molecule comprise sequence corresponding to a different genomic locus in the host cell as compared to each other first and second homology arms on each other circular molecule.

42. The method of any one of embodiments 1-40, wherein the first and second homology arms on each circular molecule comprise sequence corresponding to the same genomic locus in the host cell as compared to each other first and second homology arms on each other circular molecule.

43. The method of any one of the above embodiments, wherein each of the one or more payload sequences in a circular molecule is different from the one or more payload sequences in each other circular molecule.

44. The method of any one of embodiments 1-42, wherein each of the one or more payload sequences in a circular molecule is the same as the one or more payload sequences in each other circular molecule.

45. The method of any one of the above embodiments, wherein the introducing in step (c) entails performing double-crossover integration of the pool of linear insert polynucleotides in the host cell.

46. The method of any one of embodiments 1-44, wherein the introducing in step (c) entails performing CRISPR-mediated homology directed repair with the pool of linear insert polynucleotides and a pool of guide RNAs (gRNA) introduced into the host cell.

47. The method of embodiment 46, wherein each of the gRNAs in the pool of gRNAs comprise sequence complementary to a genomic locus targeted by the first and second homology arms in one or more of the linear insert polynucleotides present in the pool of linear insert polynucleotides.

48. The method of embodiment 46, wherein the pool of gRNAs comprises gRNAs that target or bind the genomic loci targeted by each of the linear insert polynucleotides in the pool of linear insert polynucleotides.

49. The method of embodiment 48, wherein the pool of gRNAs comprises gRNAs that target or bind genomic loci targeted by a subset of linear insert polynucleotides in the pool of linear insert polynucleotides.

50. The method of any one of embodiments 1-44, wherein the introducing in step (c) entails performing lambda red mediated integration of the pool of linear insert polynucleotides in the host cell.

51. The method of any one of the above embodiments, wherein the host cell is selected from the group consisting of a bacterial cell, an algal cell, a plant cell, a fungal cell, an insect cell and a mammalian cell.

52. The method of embodiment 51, wherein the host cell is a bacterial cell.

53. The method of embodiment 52, wherein the bacterial cell is selected from Escherichia coli and Corynebacterium glutamicum.

54. The method of any one of embodiments 51-53, wherein the Corynebacterium glutamicum is selected from Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofennentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

55. The method of any one of embodiments 51-53, wherein the Escherichia coli is selected from Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli O157:H7, E. coli O104:H4, Escherichia coli O121, Escherichia coli O104:H21, Escherichia coli K1, and Escherichia coli NC101.

56. The method of embodiment 51, wherein the host cell is a fungal cell.

57. The method of embodiment 56, wherein the fungal cell is selected from Saccharomyces cerevisiae and Pichia pastoris.

58. The method of embodiment 56, wherein the fungal cell is a filamentous fungal cell.

59. The method of embodiment 58, wherein the filamentous fungal cell is selected from Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella species or teleomorphs, or anamorphs, and synonyms or taxonomic equivalents thereof.

60. The method of embodiment 58 or 59, wherein the filamentous fungal host cell is Aspergillus niger.

61. A composition comprising a pool of insert polynucleotides, and a pool of targeting polynucleotides, wherein each insert polynucleotide in the pool of insert polynucleotides comprises one or more payload sequences, wherein, for each insert polynucleotide, the composition comprises at least one targeting polynucleotide from the pool of targeting polynucleotides, wherein the at least one targeting polynucleotide comprises from 5′ to 3′, a first assembly overlap sequence comprising sequence complementary to a distal or 3′ end of the insert polynucleotide, a first homology arm, a linearization sequence, a second homology arm and a second assembly overlap sequence comprising sequence complementary to a reverse complement of a proximal or 5′ end of the insert polynucleotide, wherein the first homology arm and the second homology arm comprise sequence complementary to a genomic locus in a host cell.

62. The composition of embodiment 61, further comprising a pool of reverse primers, wherein, for each insert polynucleotide, the composition comprises at least one targeting polynucleotide from the pool of targeting polynucleotides and a reverse primer from the pool of reverse primers, wherein the at least one targeting polynucleotide comprises from 5′ to 3′, a first assembly overlap sequence comprising sequence complementary to a distal or 3′ end of the insert polynucleotide, the first homology arm, the linearization sequence, the second homology arm and a second assembly overlap sequence comprising sequence complementary to a reverse complement of a proximal or 5′ end of the insert polynucleotide, and wherein the reverse primer comprises sequence complementary to the distal or 3′ end of the insert polynucleotide, and wherein the pool of targeting polynucleotides is a pool of forward primers.

63. The composition of embodiment 61, wherein each insert polynucleotide in the pool of insert polynucleotides comprises a recognition sequence for the Type IIS restriction enzyme on both the insert polynucleotide'sproximal or 5′ end and distal or 3′ end which upon digestion with the Type IIS restriction enzyme generates a proximal overhang and distal overhang, respectively, and wherein, for each insert polynucleotide, the mixture comprises at least one targeting polynucleotide from the pool of targeting polynucleotides, wherein the first assembly overlap sequence and the second assembly overlap sequence of the targeting polynucleotide each comprise the recognition sequence for the Type IIS restriction enzyme which upon digestion with the Type IIS restriction enzyme generates an overhang in the first assembly overlap sequence compatible with the distal overhang of the insert polynucleotide as well as an overhang in the second assembly overlap sequence compatible with the proximal overhang of the insert polynucleotide.

64. The composition of embodiment 63, further comprising a Type IIS restriction enzyme and a ligase.

65. The composition of embodiment 64, wherein the Type IIS restriction enzyme is a Type IIS restriction enzyme that generates a four-base overhang.

66. The method of embodiment 65, wherein the Type IIS restriction enzyme is selected from the group consisting of BsaI, BbsI, BsmBI and Esp3I.

67. The method of any one of embodiments 64-66, wherein the ligase is a T4 DNA ligase.

68. The composition of any one of embodiments 61-67, wherein the linearization sequence comprises one or more recognition sequences for one or more site-specific nucleases.

69. The composition of embodiment 68, wherein the one or more site-specific nuclease(s) recognition sequence are for one or more of Type I restriction endonuclease(s), Type IIS restriction endonuclease(s), a meganuclease, RNA-guided nuclease(s), DNA-guided nuclease(s), zinc-finger nuclease(s), TALEN(s) or nicking enzyme(s).

70. The composition of any one of embodiments 61-67, wherein the linearization sequence comprises one or more primer binding sites that are common to each targeting polynucleotide in the pool of targeting polynucleotides.

71. The composition of embodiment 70, wherein at least one of the one or more primer binding sites in the targeting polynucleotide is common to at least one of the one or more primer binding sites in each other targeting polynucleotide in the pool of targeting polynucleotides.

72. The composition of embodiment 71, wherein the primer pair directed to one of the one or more primer binding sites located within the linearization sequence is directed to the primer binding site common to each targeting polynucleotide in the pool of targeting polynucleotides.

73. The composition of embodiment 70, wherein at least one of the one or more primer binding sites in the targeting polynucleotide is not found in any of the one or more primer binding sites in each other targeting polynucleotide in the pool of targeting polynucleotides.

74. The composition of embodiment 73, wherein the primer pair directed to one of the one or more primer binding sites located within the linearization sequence is directed to the primer binding site not found in any of the one or more primer binding sites in each other targeting polynucleotide in the pool of targeting polynucleotides.

75. The composition of embodiment 70, wherein at least one of the one or more primer binding sites in the targeting polynucleotide is common to at least one of the one or more primer binding sites in a subset of other targeting polynucleotides in the pool of targeting polynucleotides.

76. The composition of embodiment 75, wherein the primer pair directed to one of the one or more primer binding sites located within the linearization sequence is directed to the primer binding site common to the subset of other targeting polynucleotides in the pool of targeting polynucleotides.

77. The composition of any one of embodiments 61-76, wherein the first assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the distal or 3′ end of the insert polynucleotide.

78. The composition of any one of embodiments 61-77, wherein the second assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the reverse complement of the proximal or 5′ end of the insert polynucleotide.

79. The composition of any one of embodiments 61-78, wherein the distal or 3′ end of the insert polynucleotide to which the first assembly overlap sequence comprises sequence complementary thereto is found within one of the one or more payload sequences.

80. The composition of any one of embodiments 61-78, wherein the distal or 3′ end of the insert polynucleotide to which the first assembly overlap sequence comprises sequence complementary thereto is found downstream of the one or more payload sequences.

81. The composition of any one of embodiments 61-80, wherein the proximal or 5′ end of the insert polynucleotide to which the second assembly overlap sequence comprises sequence complementary to the reverse complement thereof is found within one of the one or more payload sequences.

82. The composition of any one of embodiments 61-80, wherein the proximal or 5′ end of the insert polynucleotide to which the second assembly overlap sequence comprises sequence complementary to the reverse complement thereof is found upstream of the one or more payload sequences.

83. The composition of any one of embodiments 61-82, wherein the distal or 3′ end of the insert polynucleotide to which the reverse primer comprises sequence complementary thereto is found within one of the one or more payload sequences.

84. The composition of any one of embodiments 61-82, wherein the distal or 3′ end of the insert polynucleotide to which the reverse primer comprises sequence complementary thereto is found downstream of the one or more payload sequences.

85. The composition of any one of embodiments 61-84, wherein each insert polynucleotide is present on a plasmid.

86. The composition of any one of embodiments 61-84, wherein each insert polynucleotide is a linear fragment of nucleic acid.

87. The composition of embodiment 86, wherein each linear insert polynucleotide is a gBlock.

88. The composition of embodiment 86, wherein each insert polynucleotide is single-stranded or double-stranded.

89. The composition of any one of embodiments 61-88, wherein each payload sequence is selected from the group consisting of whole or portions of promoters, genes, regulatory sequences, nucleic acid sequence encoding degrons, nucleic acid sequence encoding solubility tags, terminators, unique identifier sequence and combinations thereof.

90. The composition of any one of embodiments 61-89, wherein each payload sequence and/or targeting polynucleotide comprises a barcode sequence.

91. The composition of embodiment 90, wherein the barcode sequence comprises a sequence unique to each combination of payload sequence and first and second homology arms flanked by sequence universal to the barcode sequence present in each other payload sequence.

92. The composition of embodiment 91, wherein the sequence universal to the barcode sequence present in each other payload sequence is used for amplifying or sequencing the unique sequence in each barcode.

93. The composition of any one of embodiments 61-92, wherein the insert polynucleotide further comprises sequence for a selectable marker.

94. The composition of embodiment 93, wherein the sequence for the selectable marker is flanked by direct repeat sequences that serve to facilitate looping out of the sequence for the selectable marker.

95. The composition of embodiment 93 or 94, wherein the selectable marker is selected from the group consisting of an antibiotic resistance gene, an auxotrophic marker, a colorimetric marker, a gene for a reporter protein and a directional marker.

96. The composition of any one of embodiments 61-95, wherein the first and second homology arms on each targeting polynucleotide in the pool of targeting polynucleotides comprise sequence corresponding to a different genomic locus in the host cell as compared to each other first and second homology arms on each other targeting polynucleotides in the pool of targeting polynucleotides.

97. The composition of any one of embodiments 61-95, wherein the first and second homology arms on each targeting polynucleotide in the pool of targeting polynucleotides comprise sequence corresponding to the same genomic locus in the host cell as compared to each other first and second homology arms on each other targeting polynucleotide in the pool of targeting polynucleotides.

98. The composition of any one of embodiments 61-97, wherein each of the one or more payload sequences in an insert polynucleotide in the pool of insert polynucleotides is different from the one or more payload sequences in each other insert polynucleotide in the pool of insert polynucleotides.

99. The composition of any one of embodiments 61-97, wherein each of the one or more payload sequences in an insert polynucleotide in the pool of insert polynucleotides is the same as the one or more payload sequences in each other insert polynucleotide in the pool of insert polynucleotides.

100. The composition of any one of embodiments 61-99, wherein the composition further comprises a pool of gRNAs.

101. The method of embodiment 100, wherein each of the gRNAs in the pool of gRNAs comprise sequence complementary to a genomic locus targeted by the first and second homology arms in one or more of the target polynucleotides present in the pool of targeting polynucleotides.

102. The method of embodiment 100, wherein the pool of gRNAs comprises gRNAs that target or bind the genomic loci targeted by each of the target polynucleotides in the pool of target polynucleotides.

103. The method of embodiment 100, wherein the pool of gRNAs comprises gRNAs that target or bind genomic loci targeted by a subset of target polynucleotides in the pool of target polynucleotides.

104. The composition of any one of embodiments 61-103, wherein the host cell is selected from the group consisting of a bacterial cell, an algal cell, a plant cell, a fungal cell, an insect cell and a mammalian cell.

105. The composition of embodiment 104, wherein the host cell is a bacterial cell.

106. The composition of embodiment 105, wherein the bacterial cell is selected from Escherichia coli and Corynebacterium glutamicum.

107. The composition of any one of embodiments 104-106, wherein the Corynebacterium glutamicum is selected from Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofennentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

108. The composition of any one of embodiments 104-106, wherein the Escherichia coli is selected from Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli O157:H7, E. coli O104:H4, Escherichia coli O121, Escherichia coli O104:H21, Escherichia coli K1, and Escherichia coli NC101.

109. The composition of embodiment 104, wherein the host cell is a fungal cell.

110. The composition of embodiment 109, wherein the fungal cell is selected from Saccharomyces cerevisiae and Pichia pastoris.

111. The composition of embodiment 109, wherein the fungal cell is a filamentous fungal cell.

112. The composition of embodiment 111, wherein the filamentous fungal cell is selected from Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella species or teleomorphs, or anamorphs, and synonyms or taxonomic equivalents thereof.

113. The composition of embodiment 111 or 112, wherein the filamentous fungal host cell is Aspergillus niger.

*******

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary, to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

In addition, the following particular applications are incorporated herein by reference: U.S. application Ser. No. 15/396,230 (U.S. Pub. No. US 2017/0159045 A1) filed on Dec. 30, 20016; PCT/US2016/065465 (WO 2017/100377 A1) filed on Dec. 7, 2016; U.S. application Ser. No. 16/669,940 (U.S. Pub. No. US 2020/0131508 Al) filed on Oct. 31, 2019; and PCMS2019/059051 (WO 2020/092704 A1) filed on Oct. 31, 2019.

What is claimed:

1. A method for genetically editing a host cell, the method comprising:
(a) assembling a pool of insert polynucleotides and a pool of targeting polynucleotides into a pool of circular molecules, wherein each circular molecule from the pool of circular molecules comprises one or more payload sequences flanked by a first homology arm 5′ to the one or more payload sequences and a second homology arm 3′ to the one or more payload sequences and a linearization sequence that is located between both the first and second homology arms;
(b) linearizing each of the circular molecules from the pool of circular molecules via the linearization sequence , thereby generating a pool of linear insert polynucleotides, wherein each linear insert polynucleotide in the pool comprises from 5′ to 3′ a first homology arm, one or more payload sequences and a second homology arm, wherein the first homology arm and the second homology arm comprise sequence complementary to a genomic locus in a host cell; and
(c) introducing the pool of linear insert polynucleotides into the host cell, thereby genetically editing the host cell.

2. The method of claim 1, wherein the assembling of step (a) comprises:
(i) providing a pool of reverse primers along with the pool of insert polynucleotides and the pool of targeting polynucleotides, wherein the pool of targeting polynucleotides are forward primers, thereby generating a mixture comprising the pool of insert polynucleotides, the pool of forward primers and the pool of reverse primers, wherein, for each insert polynucleotide, the mixture comprises at least one forward primer from the pool of forward primers and a reverse primer from the pool of reverse primers, wherein the at least one forward primer comprises from 5′ to 3′, a first assembly overlap sequence comprising sequence complementary to a distal or 3′ end of the insert polynucleotide, the first homology arm, the linearization sequence, the second homology arm and a second assembly overlap sequence comprising sequence complementary to a reverse complement of a proximal or 5′ end of the insert polynucleotide, and wherein the reverse primer comprises sequence complementary to the distal or 3′ end of the insert polynucleotide;
(ii) performing a polymerase chain reaction (PCR) on the mixture, wherein, for each insert polynucleotide, the PCR generates a PCR product comprising from 5′ to 3′, the first assembly overlap sequence, the first homology arm, the linearization sequence, the second homology arm and the one or more payload sequences; and
(iii) circularizing the PCR products from step (ii) via an assembly method selected from the group consisting of splicing and overlap-extension PCR (SOE-PCR), Uracil-specific excision reagent (USER) cloning, restriction-ligation, scarless restriction-ligation, blunt-end ligation, an overlap based assembly method and recombination-based method, or any other enzymatic or chemical method of joining two DNA molecules.

3. The method of claim 1, wherein the assembling of step (a) comprises directly performing an assembly method on a mixture comprising the pool of insert polynucleotides and the pool of targeting polynucleotides, wherein, for each insert polynucleotide, the mixture comprises at least one targeting polynucleotide from the pool of targeting polynucleotides, wherein the at least one targeting polynucleotide comprises from 5′ to 3′, a first assembly overlap sequence comprising sequence complementary to a distal or 3′ end of the insert polynucleotide, the first homology arm, the linearization sequence, the second homology arm and a second assembly overlap sequence comprising sequence complementary to a reverse complement of a proximal or 5′ end of the insert polynucleotide, and wherein the assembly method is selected from selected from the group consisting of splicing and overlap-extension PCR (SOE-PCR), Uracil-specific excision reagent (USER) cloning, restriction-ligation, blunt-end ligation, overlap based assembly method and recombination-based method, or any other enzymatic or chemical method of joining two DNA molecules.

4. The method of claim 3, wherein the assembling method is an overlap based assembly method utilizing a Type IIS restriction enzyme and a ligase, wherein each insert polynucleotide in the pool of insert polynucleotides comprises a recognition sequence for the Type IIS restriction enzyme on both the insert polynucleotide's proximal or 5′ end and distal or 3′ end which upon digestion with the Type IIS restriction enzyme generates a proximal overhang and distal overhang, respectively, and wherein, for each insert polynucleotide, the mixture comprises at least one targeting polynucleotide from the pool of targeting polynucleotides, wherein the first assembly overlap sequence and the second assembly overlap sequence of the targeting polynucleotide each comprise the recognition sequence for the Type IIS restriction enzyme which upon digestion with the Type IIS restriction enzyme generates an overhang in the first assembly overlap sequence compatible with the distal overhang of the insert polynucleotide as well as an overhang in the second assembly overlap sequence compatible with the proximal overhang of the insert polynucleotide.

5. The method of claim 4, wherein the Type IIS restriction enzyme is a Type IIS restriction enzyme that generates a four-base overhang.

6. The method of claim 5, wherein the Type IIS restriction enzyme is selected from the group consisting of BsaI, BbsI, BsmBI and Esp3I.

7. The method of claim 4, wherein the ligase is a T4 DNA ligase.

8. The method of any one or claims 3-7, wherein each targeting polynucleotide in the pool of targeting polynucleotides is subjected to a primer extension reaction using a reverse primer comprising sequence that binds to the second assembly overlap sequence, thereby generating a double-stranded (ds) targeted polynucleotide.

9. The method of claim 8, wherein the top or sense strand of each ds targeting polynucleotide comprises, from 5′ to 3′, the first assembly overlap sequence comprising sequence complementary to the distal or 3′ end of the insert polynucleotide, the first homology arm, the linearization sequence, the second homology arm and the second assembly overlap sequence comprising sequence complementary to the reverse complement of the proximal or 3′ end of the insert polynucleotide.

10. The method of claim 1, wherein the linearizing of step (b) comprises rolling circle amplification (RCA) of each circular molecule from the pool of circular molecules, wherein the RCA of each circular molecule produces a concatenated linear product comprising repeated units each separated by the linearization sequence, wherein each of the repeated units comprises the insert polynucleotide flanked upstream by the first homology arm and downstream by the second homology arm, wherein the insert polynucleotides are released from the concatenated linear product via the linearization sequence present between each repeated unit, thereby generating the pool of linear insert polynucleotides.

11. The method of claim 1, wherein the linearization sequence comprises one or more recognition sequences for one or more site-specific nucleases.

12. The method of claim 11, wherein the linearizing comprises digesting the one or more recognition sequences with one or more site-specific nuclease(s) that recognize the one or more site-specific nuclease recognition sequence(s).

13. The method of claim 11 or 12, wherein the one or more site-specific nuclease(s) recognition sequence are for one or more of Type I restriction endonuclease(s), Type IIS restriction endonuclease(s), meganuclease, RNA-guided nuclease(s), DNA-guided nuclease(s), zinc-finger nuclease(s), TALEN(s) or nicking enzyme(s).

14. The method of claim 1, wherein the linearization sequence comprises one or more primer binding sites that are common to each targeting polynucleotide in the pool of targeting polynucleotides.

15. The method of claim 14, wherein the linearizing of step (b) comprises performing a PCR using a primer pair directed to one of the one or more primer binding sites located within the linearization sequence.

16. The method of claim 14 or 15, wherein at least one of the one or more primer binding sites in the targeting polynucleotide is common to at least one of the one or more primer binding sites in each other targeting polynucleotide in the pool of targeting polynucleotides.

17. The method of claim 16, wherein the primer pair directed to one of the one or more primer binding sites located within the linearization sequence in step (b) is directed to the primer binding site common to each targeting polynucleotide in the pool of targeting polynucleotides.

18. The method of claim 14 or 15, wherein at least one of the one or more primer binding sites in the targeting polynucleotide is not found in any of the one or more primer binding sites in each other targeting polynucleotide in the pool of targeting polynucleotides.

19. The method of claim 18, wherein the primer pair directed to one of the one or more primer binding sites located within the linearization sequence in step (b) is directed to the primer binding site not found in any of the one or more primer binding sites in each other targeting polynucleotide in the pool of targeting polynucleotides.

20. The method of claim 14 or 15, wherein at least one of the one or more primer binding sites in the targeting polynucleotide is common to at least one of the one or more primer binding sites in a subset of other targeting polynucleotides in the pool of targeting polynucleotides.

21. The method of claim 20, wherein the primer pair directed to one of the one or more primer binding sites located within the linearization sequence in step (b) is directed to the primer binding site common to the subset of other targeting polynucleotides in the pool of targeting polynucleotides.

22. The method of claim 2, wherein the first assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the distal or 3′ end of the insert polynucleotide.

23. The method of claim 2, wherein the second assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the reverse complement of the proximal or 5′ end of the insert polynucleotide.

24. The method of claim 2, wherein the distal or 3′ end of the insert polynucleotide to which the first assembly overlap sequence comprises sequence complementary thereto is found within one of the one or more payload sequences.

25. The method of claim 2, wherein the distal or 3′ end of the insert polynucleotide to which the first assembly overlap sequence comprises sequence complementary thereto is found downstream of the one or more payload sequences.

26. The method of claim 2, wherein the proximal or 5′ end of the insert polynucleotide to which the second assembly overlap sequence comprises sequence complementary to the reverse complement thereof is found within one of the one or more payload sequences.

27. The method of claim 2, wherein the proximal or 5′ end of the insert polynucleotide to which the second assembly overlap sequence comprises sequence complementary to the reverse complement thereof is found upstream of the one or more payload sequences.

28. The method of claim 2, wherein the distal or 3′ end of the insert polynucleotide to which the reverse primer comprises sequence complementary thereto is found within one of the one or more payload sequences.

29. The method of claim 2, wherein the distal or 3′ end of the insert polynucleotide to which the reverse primer comprises sequence complementary thereto is found downstream of the one or more payload sequences.

30. The method of claim 1, wherein each insert polynucleotide is present on a plasmid.

31. The method of claim 1, wherein each insert polynucleotide is a linear fragment of nucleic acid.

32. The method of claim 31, wherein each linear insert polynucleotide is a gBlock.

33. The method of claim 31, wherein each insert polynucleotide is single-stranded or double-stranded.

34. The method of claim 1, wherein each payload sequence is selected from the group consisting of whole or portions of promoters, genes, regulatory sequences, nucleic acid sequence encoding degrons, nucleic acid sequence encoding solubility tags, terminators, unique identifier sequence, and combinations thereof.

35. The method of claim 1, wherein each payload sequence and/or targeting polynucleotide further comprises a barcode sequence.

36. The method of claim 35, wherein the barcode sequence comprises a sequence unique to each combination of payload sequence and first and second homology arms flanked by sequence universal to the barcode sequence present in each other payload sequence.

37. The method of claim 36, wherein the sequence universal to the barcode sequence present in each other payload sequence is used for amplifying or sequencing the unique sequence in each barcode.

38. The method of claim 1, wherein the insert polynucleotide further comprises sequence for a selectable marker.

39. The method of claim 38, wherein the sequence for the selectable marker is flanked by direct repeat sequences that serve to facilitate looping out of the sequence for the selectable marker.

40. The method of claim 38 or 39, wherein the selectable marker is selected from the group consisting of an antibiotic resistance gene, an auxotrophic marker, a colorimetric marker, a gene for a reporter protein and a directional marker.

41. The method of claim 1, wherein the first and second homology arms on each circular molecule comprise sequence corresponding to a different genomic locus in the host cell as compared to each other first and second homology arms on each other circular molecule.

42. The method of claim 1, wherein the first and second homology arms on each circular molecule comprise sequence corresponding to the same genomic locus in the host cell as compared to each other first and second homology arms on each other circular molecule.

43. The method of claim 1, wherein each of the one or more payload sequences in a circular molecule is different from the one or more payload sequences in each other circular molecule.

44. The method of claim 1, wherein each of the one or more payload sequences in a circular molecule is the same as the one or more payload sequences in each other circular molecule.

45. The method of claim 1, wherein the introducing in step (c) entails performing double-crossover integration of the pool of linear insert polynucleotides in the host cell.

46. The method of claim 1, wherein the introducing in step (c) entails performing CRISPR-mediated homology directed repair with the pool of linear insert polynucleotides and a pool of guide RNAs (gRNA) introduced into the host cell.

47. The method of claim 46, wherein each of the gRNAs in the pool of gRNAs comprise sequence complementary to a genomic locus targeted by the first and second homology arms in one or more of the linear insert polynucleotides present in the pool of linear insert polynucleotides.

48. The method of claim 46, wherein the pool of gRNAs comprises gRNAs that target or bind the genomic loci targeted by each of the linear insert polynucleotides in the pool of linear insert polynucleotides.

49. The method of claim 48, wherein the pool of gRNAs comprises gRNAs that target or bind genomic loci targeted by a subset of linear insert polynucleotides in the pool of linear insert polynucleotides.

50. The method of claim 1, wherein the introducing in step (c) entails performing lambda red mediated integration of the pool of linear insert polynucleotides in the host cell.

51. The method of claim 1, wherein the host cell is selected from the group consisting of a bacterial cell, an algal cell, a plant cell, a fungal cell, an insect cell and a mammalian cell.

52. The method of claim 51, wherein the host cell is a bacterial cell.

53. The method of claim 52, wherein the bacterial cell is selected from Escherichia coli and Corynebacterium glutamicum.

54. The method of any one of claims 51-53, wherein the Corynebacterium glutamicum is selected from Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

55. The method of any one of claims 51-53, wherein the Escherichia coli is selected from Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli O157:H7, E. coli O104:H4, Escherichia coli O121, Escherichia coli O104:H21, Escherichia coli K1, and Escherichia coli NC101.

56. The method of claim 51, wherein the host cell is a fungal cell.

57. The method of claim 56, wherein the fungal cell is selected from Saccharomyces cerevisiae and Pichia pastoris.

58. The method of claim 56, wherein the fungal cell is a filamentous fungal cell.

59. The method of claim 58, wherein the filamentous fungal cell is selected from Achlya, Acremonium, Aspergillus, Aureobasidium, Bjerkandera, Ceriporiopsis, Cephalosporium, Chrysosporium, Cochhobolus, Corynascus, Cryphonectria, Cryptococcus, Coprinus, Coriolus, Diplodia, Endothis, Fusarium, Gibberella, Ghocladium, Humicola, Hypocrea, Mycehophthora (e.g., Mycehophthora thermophila), Mucor, Neurospora, Penicillium, Podospora, Phlebia, Piromyces, Pyricularia, Rhizomucor, Rhizopus, Schizophyllum, Scyta/idium, Sporotrichum, Talaromyces, Thermoascus, Thielavia, Tramates, Tolypocladium, Trichoderma, Verticillium, Volvariella species or teleomorphs, or anamorphs, and synonyms or taxonomic equivalents thereof.

60. The method of claim 58 or 59, wherein the filamentous fungal host cell is Aspergillus niger.

61. A composition comprising a pool of insert polynucleotides, and a pool of targeting polynucleotides, wherein each insert polynucleotide in the pool of insert polynucleotides comprises one or more payload sequences, wherein, for each insert polynucleotide, the composition comprises at least one targeting polynucleotide from the pool of targeting polynucleotides, wherein the at least one targeting polynucleotide comprises from 5′ to 3′, a first assembly overlap sequence comprising sequence complementary to a distal or 3′ end of the insert polynucleotide, a first homology arm, a linearization sequence, a second homology arm and a second assembly overlap sequence comprising sequence complementary to a reverse complement of a proximal or 5′ end of the insert polynucleotide, wherein the first homology arm and the second homology arm comprise sequence complementary to a genomic locus in a host cell.

62. The composition of claim 61, further comprising a pool of reverse primers, wherein, for each insert polynucleotide, the composition comprises at least one targeting polynucleotide from the pool of targeting polynucleotides and a reverse primer from the pool of reverse primers, wherein the at least one targeting polynucleotide comprises from 5′ to 3′, a first assembly overlap sequence comprising sequence complementary to a distal or 3′ end of the insert polynucleotide, the first homology arm, the linearization sequence, the second homology arm and a second assembly overlap sequence comprising sequence complementary to a reverse complement of a proximal or 5′ end of the insert polynucleotide, and wherein the reverse primer comprises sequence complementary to the distal or 3′ end of the insert polynucleotide, and wherein the pool of targeting polynucleotides is a pool of forward primers.

63. The composition of claim 61, wherein each insert polynucleotide in the pool of insert polynucleotides comprises a recognition sequence for the Type IIS restriction enzyme on both the insert polynucleotide'sproximal or 5′ end and distal or 3′ end which upon digestion with the Type IIS restriction enzyme generates a proximal overhang and distal overhang, respectively, and wherein, for each insert polynucleotide, the mixture comprises at least one targeting polynucleotide from the pool of targeting polynucleotides, wherein the first assembly overlap sequence and the second assembly overlap sequence of the targeting polynucleotide each comprise the recognition sequence for the Type IIS restriction enzyme which upon digestion with the Type IIS restriction enzyme generates an overhang in the first assembly overlap sequence compatible with the distal overhang of the insert polynucleotide as well as an overhang in the second assembly overlap sequence compatible with the proximal overhang of the insert polynucleotide.

64. The composition of claim 63, further comprising a Type IIS restriction enzyme and a ligase.

65. The composition of claim 64, wherein the Type IIS restriction enzyme is a Type IIS restriction enzyme that generates a four-base overhang.

66. The method of claim 65, wherein the Type IIS restriction enzyme is selected from the group consisting of BsaI, BbsI, BsmBI and Esp3I.

67. The method of claim 64, wherein the ligase is a T4 DNA ligase.

68. The composition of any one of claims 61-67, wherein the linearization sequence comprises one or more recognition sequences for one or more site-specific nucleases.

69. The composition of claim 68, wherein the one or more site-specific nuclease(s) recognition sequence are for one or more of Type I restriction endonuclease(s), Type IIS restriction endonuclease(s), a meganuclease, RNA-guided nuclease(s), DNA-guided nuclease(s), zinc-finger nuclease(s), TALEN(s) or nicking enzyme(s).

70. The composition of any one of claims 61-67, wherein the linearization sequence comprises one or more primer binding sites that are common to each targeting polynucleotide in the pool of targeting polynucleotides.

71. The composition of claim 70, wherein at least one of the one or more primer binding sites in the targeting polynucleotide is common to at least one of the one or more primer binding sites in each other targeting polynucleotide in the pool of targeting polynucleotides.

72. The composition of claim 71, wherein the primer pair directed to one of the one or more primer binding sites located within the linearization sequence is directed to the primer binding site common to each targeting polynucleotide in the pool of targeting polynucleotides.

73. The composition of claim 70, wherein at least one of the one or more primer binding sites in the targeting polynucleotide is not found in any of the one or more primer binding sites in each other targeting polynucleotide in the pool of targeting polynucleotides.

74. The composition of claim 73, wherein the primer pair directed to one of the one or more primer binding sites located within the linearization sequence is directed to the primer binding site not found in any of the one or more primer binding sites in each other targeting polynucleotide in the pool of targeting polynucleotides.

75. The composition of claim 70, wherein at least one of the one or more primer binding sites in the targeting polynucleotide is common to at least one of the one or more primer binding sites in a subset of other targeting polynucleotides in the pool of targeting polynucleotides.

76. The composition of claim 75, wherein the primer pair directed to one of the one or more primer binding sites located within the linearization sequence is directed to the primer binding site common to the subset of other targeting polynucleotides in the pool of targeting polynucleotides.

77. The composition of claim 61, wherein the first assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the distal or 3′ end of the insert polynucleotide.

78. The composition of claim 61, wherein the second assembly overlap sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides that are complementary to the reverse complement of the proximal or 5′ end of the insert polynucleotide.

79. The composition of claim 61, wherein the distal or 3′ end of the insert polynucleotide to which the first assembly overlap sequence comprises sequence complementary thereto is found within one of the one or more payload sequences.

80. The composition of claim 61, wherein the distal or 3′ end of the insert polynucleotide to which the first assembly overlap sequence comprises sequence complementary thereto is found downstream of the one or more payload sequences.

81. The composition of claim 61, wherein the proximal or 5′ end of the insert polynucleotide to which the second assembly overlap sequence comprises sequence complementary to the reverse complement thereof is found within one of the one or more payload sequences.

82. The composition of claim 61, wherein the proximal or 5′ end of the insert polynucleotide to which the second assembly overlap sequence comprises sequence complementary to the reverse complement thereof is found upstream of the one or more payload sequences.

83. The composition of claim 61, wherein the distal or 3′ end of the insert polynucleotide to which the reverse primer comprises sequence complementary thereto is found within one of the one or more payload sequences.

84. The composition of claim 61, wherein the distal or 3′ end of the insert polynucleotide to which the reverse primer comprises sequence complementary thereto is found downstream of the one or more payload sequences.

85. The composition of claim 61, wherein each insert polynucleotide is present on a plasmid.

86. The composition of claim 61, wherein each insert polynucleotide is a linear fragment of nucleic acid.

87. The composition of claim 86, wherein each linear insert polynucleotide is a gBlock.

88. The composition of claim 86, wherein each insert polynucleotide is single-stranded or double-stranded.

89. The composition of claim 61, wherein each payload sequence is selected from the group consisting of whole or portions of promoters, genes, regulatory sequences, nucleic acid sequence encoding degrons, nucleic acid sequence encoding solubility tags, terminators, unique identifier sequence and combinations thereof.

90. The composition of claim 61, wherein each payload sequence and/or targeting polynucleotide comprises a barcode sequence.

91. The composition of claim 90, wherein the barcode sequence comprises a sequence unique to each combination of payload sequence and first and second homology arms flanked by sequence universal to the barcode sequence present in each other payload sequence.

92. The composition of claim 91, wherein the sequence universal to the barcode sequence present in each other payload sequence is used for amplifying or sequencing the unique sequence in each barcode.

93. The composition of claim 61, wherein the insert polynucleotide further comprises sequence for a selectable marker.

94. The composition of claim 93, wherein the sequence for the selectable marker is flanked by direct repeat sequences that serve to facilitate looping out of the sequence for the selectable marker.

95. The composition of claim 93 or 94, wherein the selectable marker is selected from the group consisting of an antibiotic resistance gene, an auxotrophic marker, a colorimetric marker, a gene for a reporter protein and a directional marker.

96. The composition of claim 61, wherein the first and second homology arms on each targeting polynucleotide in the pool of targeting polynucleotides comprise sequence corresponding to a different genomic locus in the host cell as compared to each other first and second homology arms on each other targeting polynucleotides in the pool of targeting polynucleotides.

97. The composition of claim 61, wherein the first and second homology arms on each targeting polynucleotide in the pool of targeting polynucleotides comprise sequence corresponding to the same genomic locus in the host cell as compared to each other first and second homology arms on each other targeting polynucleotide in the pool of targeting polynucleotides.

98. The composition of claim 61, wherein each of the one or more payload sequences in an insert polynucleotide in the pool of insert polynucleotides is different from the one or more payload sequences in each other insert polynucleotide in the pool of insert polynucleotides.

99. The composition of claim 61, wherein each of the one or more payload sequences in an insert polynucleotide in the pool of insert polynucleotides is the same as the one or more payload sequences in each other insert polynucleotide in the pool of insert polynucleotides.

100. The composition of claim 61, wherein the composition further comprises a pool of gRNAs.

101. The method of claim 100, wherein each of the gRNAs in the pool of gRNAs comprise sequence complementary to a genomic locus targeted by the first and second homology arms in one or more of the target polynucleotides present in the pool of targeting polynucleotides.

102. The method of claim 100, wherein the pool of gRNAs comprises gRNAs that target or bind the genomic loci targeted by each of the target polynucleotides in the pool of target polynucleotides.

103. The method of claim 100, wherein the pool of gRNAs comprises gRNAs that target or bind genomic loci targeted by a subset of target polynucleotides in the pool of target polynucleotides.

104. The composition of claim 61, wherein the host cell is selected from the group consisting of a bacterial cell, an algal cell, a plant cell, a fungal cell, an insect cell and a mammalian cell.

105. The composition of claim 104, wherein the host cell is a bacterial cell.

106. The composition of claim 105, wherein the bacterial cell is selected from Escherichia coli and Corynebacterium glutamicum.

107. The composition of any one of claims 104-106, wherein the Corynebacterium glutamicum is selected from Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

108. The composition of any one of claims 104-106, wherein the Escherichia coli is selected from Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli O157:H7, E. coli O104:H4, Escherichia coli O121, Escherichia coli O104:H21, Escherichia coli K1, and Escherichia coli NC101.

109. The composition of claim 104, wherein the host cell is a fungal cell.

110. The composition of claim 109, wherein the fungal cell is selected from Saccharomyces cerevisiae and Pichia pastoris.

111. The composition of claim 109, wherein the fungal cell is a filamentous fungal cell.

112. The composition of claim 111, wherein the filamentous fungal cell is selected from Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Tramates, Tolypocladium, Trichoderma, Verticillium, Volvariella species or teleomorphs, or anamorphs, and synonyms or taxonomic equivalents thereof.

113. The composition of claim 111 or 112, wherein the filamentous fungal host cell is Aspergillus niger.
